SEQUENCE LISTING (116 sequences)

SEQ ID NO: 1 - DNA, 2481 bp, Camelina sativa
  misc_feature (2288)..(2288): n is a, c, g, or t
atggatataagtaatgaagccagtgttgatcccttttccattggaccaacatcgatcatgggtcgaaccattgctttcagagtcttgttctgtagatcaatggctcagcttaggcgtgatctttttcgcttcttgctgcattggttccttaggtttaagctgaccgtatcaccgtttgtgtcgtggtttcatcctcggaacccgcaagggatcttagcagttgttacaatcattgctttcgtgttgaaacgttacaccaatgtgaagataaaggctgaaatggcttacaggaggaaattttggaggaacatgatgcggacggctttgacttatgaggaatgggctcatgctgctaagatgttagagaaggagacgccaaagctgaatgaatctgatctttatgatgaagagttggttaagaacaagcttcaggagcttcgtcatcgtcgccaagaaggttcattgagagacatcatgttttgtatgagagctgatctcgtgaggaatcttggtaatatgtgtaactcggagcttcataaaggtagacttcaggttcctagacatatcaaagagtatatcgatgaggtctctactcagttgagaatggtctgtaactctgactcagaggagctttctttggaagagaagctttcctttatgcacgaaacacggcatgcctttggtagaacggctttgcttttgagtggtggggcttctcttggtgcgtttcatgttggtgtggttaggactttggttgagcataagcttttacctcggatcattgctggttctagtgtcggatctattatttgtgctgttgtggcctcaaggtcttggccggagctacaaagtttctttgagaattctttgcattctttacagttctttgatcagcttggaggcgttttctcaattgtgaaacgagtaatgacacaaggcgctctacacgatatcagacagttacagtgtatgcttagaaacctcacaagcaatctcacattccaagaagcttatgacatgacaggaaggatacttgggataaccgtttgttccccaagaaagcatgaacctcctcggtgtcttaactatttgacttcgcctcatgtggttatatggagcgcagtgactgcttcttgtgctttccccggtctctttgaagctcaagagctaatggctaaagatcgaagtggagagattgtgccatatcatccacctttcaatttgggtccagaagtaggcactaaatcatctggacgtcgatggagagatggtagtttggaggttgatttaccaatgatgcagctgaaggaactgttcaatgtcaatcattttattgtgagtcaagccaatcctcacattgctcccttactccgtctgaaagatttagttcgagcttatggtggtagatttgcagctaagctcgcgcatctagtggagatggaggtcaaacatagatgcaaccaggtattagagcttggttttccactcggtggactcgcaaagctatttgctcaggagtgggaaggtgatgtaacagttgtaatgcctgctactcttgctcagtactcgaagattatacaaaacccgactcatctcgatcttcagaaagcggctaaccaaggaagaagatgcacttgggagaagctttcagccataaaatcaaactgcgggatcgagcttgctcttgatgattctgtcgctatacttaaccatatgcgtaggctcaagaaaagcgccgagagagccgccagtgccacgtcatcgtctcatcatggattagcttcaaccaccagattcaatgcttctagaagaatcccatcttggaacgtcattgctagagagaactcaacgggttcgctcgatgatctagccgctgacaacaacaatctccatgcttcttcgggcaggaatctaagcgacagtgaaacagagagtgtggaactgagttcttcttggacaagaactggtggacctttgatgagaacagcttctgctaataagttcattgacttcgttcagtctcttgatatcgacattgcattggcgagagggttcagtagcagtcccaattctccagcagttcctcctggtggtccgtttagtccaagcgcgagatccatgtctgctcattccgatagcgaaccaaacagcaacagcaacaacaacactacttcaagaataacagtgactgaaggtgatcttctacagcctgagagaacaagtaacgggtttcttgggggcgttttctcaatcgtgaaacgagtaatgacacaaggcgctctacacgncgagttaccagagagcgtacagcttgatataccggagaaagagatggataatagctctgtatccgaacatgaagtagacgatgacgatgaacaagaagataataacggtgcaacggtctcgagtccggttactgaatcttcagaagattccggtttacaagaaccggtgtctggtagtgttatagatggttag

SEQ ID NO: 2 - DNA, 2484 bp, Camelina sativa
atggatataagtaatgaagccagtgttgatcccttttcgattggaccaacatcgatcatgggtcgaaccattgctttcagagtcttgttctgtagatctatggcacagcttaggcgtgatctttttcgctttttgctgcattggttccttaggtttaagctgactgtatcaccgtttttgtcgtggtttcatcctcggaacccacaagggatcttagcagtggttacaatcattgctttcttgttgaaacgttacaccaatgtgaagataaaagcggaaatggcttacaggaggaaattttggaggaacatgatgcggacggctttgacttatgaggaatgggctcatgctgctaagatgttggagaaggagacgccgaagctgaatgaatctgatctttatgatgaagagttggttaagaacaagcttcaggagcttcgtcatcgtcgccaagaaggttcgcttagagacatcatgttttgtatgagagctgatctcgtgaggaatcttggtaatatgtgtaactcggagcttcataaaggtagacttcaggttcctagacatatcaaagagtatatcgatgaggtctctactcagttgagaatggtctgtaactctgactcagaggagctttctttggaagagaagctttcctttatgcacgaaacacggcatgcctttggtagaacggctttgcttttgagtggtggggcttctcttggtgcgtttcatgttggtgtggttaggactttggttgagcataagcttttacctcggatcattgctggttctagtgtcggatccataatttgtgctgttgtagcctcaaggtcttggcccgagctacaaagtttctttgagaattctttgcattctttacagttctttgatcagcttggaggcgttttctcaatcgtgaaacgagtaatgacacaaggcgctctacatgatatcagacagttacagtgtatgcttagaaacctcacaagcaatctcactttccaagaagcttatgacatgacaggaaggatacttgggataaccgtttgttccccaagaaagcatgaacctcctcggtgtcttaactatttgacttcgcctcatgtggttatatggagcgcagtgactgcttcttgtgcctttcctggtctctttgaagctcaagagctaatggctaaagatcgaagtggagagatcgtgccgtatcatccacctttcaatttggatccagaagtaggcactaaatcgtctggacgccggtggagagatggtagtttggaggttgatttaccaatgatgcagcttaaggaactgttcaatgtgaatcattttattgtgagtcaagccaatcctcacattgctcccttactccgtctgaaggatttagttcgagcttatggcggtagattcgcagctaagctcgcgcatctagtggagatggaggtcaaacatagatgcaaccaggtattagagcttggttttccacttggtggacttgcaaagctatttgctcaggagtgggaaggtgatgtaacagttgtaatgcctgctactcttgctcagtactcgaagattatacaaaacccgactcatgtcgatcttcagaaagcggctaaccaaggaagaaggtgcacttgggagaagctttcagccataaaatcaaactgcgggatcgagcttgctcttgatgattcagtggctatacttaaccatatgcgtaggctcaagaaaagcgccgagagagccgccagtgccacgtcatcgtctcaccacggattagcttcaacaaccagattcaatgcttctagaagaatcccatcttggaacgtcattgccagagagaactcaacgggttcactcgatgatctagtcgctgacaacaacaatctccatgcttcttcgggcaggaatctaagcgacagtgaaacagagagtgtggaactgagttcttggacacgaactggtggacctttgatgagaacagcttctgctaataagttcattgactttgttcagtctcttgatatcgacattgcattggcaagagggttcagtagcagtcccaattctccagcagttcctcctggcggcccatttagtccaagcgcgagatccatgtctgctcattccgataacgaaccaaacagcaatagcaacaacaacactacttcaagaataacagtgactgaaggtgaccttctacagcctgagagaacaagtaacgggtttgtgttaaatgttgttaaaagagagaacttgggaatgtcatctataggggggaatcaaaatacagagttaccggagagtgtacagcttgatataccggagaaagagatggataatagctctgtatccgaacacgaagtagacgatgacgataaacaagaagataataacggtgcaacggtctcgagtccggttactgaatcttcagaagattccggtttacaagaaccggtgtctggtagtgttatagatggttag

SEQ ID NO: 3 - DNA, 2493 bp, Camelina sativa
atggatataagtaatgaagcaagtgttgatcctttttcgattggaccaacatcgatcatgggtcgaaccattgctttcagagtcttgttctgtagatcaatggcacagcttaggcgtgatctttttcggttcttgctgcattggttccttaggtttaagctgaccgtatcaccgtttgtgtcgtggtttcatcctcggaacccgcaagggatcttagcggttgttacgattattgctttcgtgttgaaacgttacaccaatgtgaagataaaagcggaaatggcttacaggaggaagttttggaggaatatgatgaggacggctttgacttatgaggaatgggctcatgctgctaagatgttggagaaggagacgccgaagctgaatgaatctgatctttatgatgaagagttggttaagaacaagcttcaggagcttcgtcatcgtcgccaagaaggttcgcttagagacatcatgttttgtatgagagctgatctcgtgaggaatcttggtaatatgtgtaactcggagcttcataaaggtagacttcaggttcctagacatatcaaagagtatatcgatgaggtctctactcagttgagaatggtctgtaactctgactcagaggagctttctttggaagagaagctttcctttatgcacgaaacacggcatgcctttggtagaacggctttgcttttgagtggtggggcttctcttggtgcgtttcatgttggtgtggttaggactttggttgagcataagcttttacctcggatcattgctggttctagtgtcggatctattatttgtgctgttgtggcctcaaggtcttggccggagctacaaagtttctttgagaattctttgcattctttacagttctttgatcagcttggaggcgttttctcaattgtgaaacgagtaatgacacaaggcgctctacacgatatcagacagttacagtgtatgcttagaaacctcacaagcaatctcacattccaagaagcttatgacatgacaggaaggatacttgggataaccgtttgttccccaagaaagcatgaacctcctcggtgtcttaactatttgacttcgcctcatgtggttatatggagcgcagtgactgcttcttgtgctttccccggtctctttgaagctcaagagctaatggctaaagatcgaagtggagagattgtgccatatcatccacctttcaatttgggtccagaagtaggcactaaatcatctggacgtcgatggagagatggtagtttggaggttgatttaccaatgatgcagctgaaggaactgttcaatgtgaatcattttattgtgagtcaagccaatcctcacattgctcccttactccgtctgaaagatttagttcgagcttatggtggtagatttgcagctaagctcgcgcatctagtggagatggaggtcaaacatagatgcaaccaggtattagagcttggttttccacttggtggacttgcaaagctatttgctcaggagtgggaaggtgatgtaacagttgtaatgcctgctactcttgctcagtactcgaagattatacaaaacccgactcatctcgatcttcagaaagcggctaaccaaggaagaagatgcacttgggagaagctttcagccataaaatcaaactgcgggatcgagcttgctcttgatgattctgtcgctatacttaaccatatgcgtaggctcaagaaaagcgccgagagagccgccagtgccacgtcatcgtctcatcatggattagcttcaaccaccagattcaatgcttctagaagaatcccatcttggaacgtcattgctagagagaactcaacgggttcgctcgatgatctagccgctgacaacaacaatctccatgcttcttcgggcaggaatctaagcgacagtgaaacagagagtgtggaactgagttcttcttggacaagaactggtggacctttgatgagaacagcttctgctaataagttcattgacttcgttcagtctcttgatatcgacattgcattggcgagagggttcagtagcagtcccaattctccagcagttcctcctggtggtccgtttagtccaagcgcgagatccatgtctgctcattccgatagcgaaccaaacagcaacagcaacaacaacactacttcaagaataacagttactgaaggtgatcttctacagcctgagagaacaagtaacgggtttgtgttaaatgttgttaaaagagagaacttgggaatgtcatctataggggggaatcaaaatacagagttaccggagagtgtacagcttgagataccggagaaagagatggataatagctctgtatccgaacatgaagtagacgatgacgatgaacatgaacaagaagataataacggtgcaacggtctcgagtccggttactgaatcttcagaagattccggtttacaagaaccgttgtctggtagtgttatagatggttag

SEQ ID NO: 4 - DNA, 20 bp, Artificial Sequence
  Synthetic construct guide sequence SDP1 #71
gacatgacaggaaggatact

SEQ ID NO: 5 - DNA, 14595 bp, Artificial Sequence
  Synthetic construct plasmid pMBXS1107
aaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgacatgacaggaaggatactgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataactaggttcgaattaattaatactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacaaggagcatcgtggaaaaagaagacgttccaaccacgtcttcaaagcaagtggattgatgtgatatctccactgacgtaagggatgacgcacaatcccactatccttcgccccaagcttgggcccaagcttgggtcgcgccccacggatggtataagaataaaggcattccgcgtgcaggattcacccgttcgcctctcaccttttcgctgtactctctcgccacacacaccccctctccagctccgttggagctccggacagcagcaggcgcggggcggtcacgtagtaagcagctctcggctccctctccccttgctccgtggatccatggattacaaggatgatgatgataaggattacaaggatgatgatgataagatggctccaaagaagaagagaaaggttggaatccacggagttccagctgctgataagaagtactctatcggacttgacatcggaaccaactctgttggatgggctgttatcaccgatgagtacaaggttccatctaagaagttcaaggttcttggaaacaccgatagacactctatcaagaagaaccttatcggtgctcttcttttcgattctggagagaccgctgaggctaccagattgaagagaaccgctagaagaagatacaccagaagaaagaacagaatctgctaccttcaggaaatcttctctaacgagatggctaaggttgatgattctttcttccacagacttgaggagtctttccttgttgaggaggataagaagcacgagagacacccaatcttcggaaacatcgttgatgaggttgcttaccacgagaagtacccaaccatctaccaccttagaaagaagttggttgattctaccgataaggctgatcttagacttatctaccttgctcttgctcacatgatcaagttcagaggacacttccttatcgagggagaccttaacccagataactctgatgttgataagttgttcatccagcttgttcagacctacaaccagcttttcgaggagaacccaatcaacgcttctggagttgatgctaaggctatcctttctgctagactttctaagtctcgtagacttgagaaccttatcgctcagcttccaggagagaagaagaacggacttttcggaaaccttatcgctctttctcttggacttaccccaaacttcaagtctaacttcgatcttgctgaggatgctaagttgcagctttctaaggatacctacgatgatgatcttgataaccttcttgctcagatcggagatcagtacgctgatcttttccttgctgctaagaacctttctgatgctatccttctttctgacatccttagagttaacaccgagatcaccaaggctccactttctgcttctatgatcaagagatacgatgagcaccaccaggatcttacccttttgaaggctcttgttagacagcagcttccagagaagtacaaggaaatcttcttcgatcagtctaagaacggatacgctggatacatcgatggaggagcttctcaggaggagttctacaagttcatcaagccaatccttgagaagatggatggaaccgaggagcttcttgttaagttgaacagagaggatcttcttagaaagcagagaaccttcgataacggatctatcccacaccagatccaccttggagagcttcacgctatccttcgtagacaggaggatttctacccattcttgaaggataacagagagaagatcgagaagatccttaccttcagaatcccatactacgttggaccacttgctagaggaaactctcgtttcgcttggatgaccagaaagtctgaggagaccatcaccccttggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttgttgataagggagcttctgctcagtctttcatcgagagaatgaccaacttcgataagaaccttccaaacgagaaggttcttccaaagcactctcttctttacgagtacttcaccgtttacaacgagcttaccaaggttaagtacgttaccgagggaatgagaaagccagctttcctttctggagagcagaagaaggctatcgttgatcttcttttcaagaccaacagaaaggttaccgttaagcagttgaaggaggattacttcaagaagatcgagtgcttcgattctgttgaaatctctggagttgaggatagattcaacgcttctcttggaacctaccacgatcttttgaagatcatcaaggataaggatttccttgataacgaggagaacgaggacatccttgaggacatcgttcttacccttacccttttcgaggatagagagatgatcgaggagagactcaagacctacgctcaccttttcgatgataaggttatgaagcagttgaagagaagaagatacaccggatggggtagactttctcgtaagttgatcaacggaatcagagataagcagtctggaaagaccatccttgatttcttgaagtctgatggattcgctaacagaaacttcatgcagcttatccacgatgattctcttaccttcaaggaggacatccagaaggctcaggtttctggacagggagattctcttcacgagcacatcgctaaccttgctggatctccagctatcaagaagggaatccttcagaccgttaaggttgttgatgagcttgttaaggttatgggtagacacaagccagagaacatcgttatcgagatggctagagagaaccagaccacccagaagggacagaagaactctcgtgagagaatgaagagaatcgaggagggaatcaaggagcttggatctcaaatcttgaaggagcacccagttgagaacacccagcttcagaacgagaagttgtacctttactaccttcagaacggaagagatatgtacgttgatcaggagcttgacatcaacagactttctgattacgatgttgatcacatcgttccacagtctttcttgaaggatgattctatcgataacaaggttcttacccgttctgataagaacagaggaaagtctgataacgttccatctgaggaggttgttaagaagatgaagaactactggagacagcttcttaacgctaagttgatcacccagagaaagttcgataaccttaccaaggctgagagaggaggactttctgagcttgataaggctggattcatcaagagacagcttgttgagaccagacagatcaccaagcacgttgctcagatccttgattctcgtatgaacaccaagtacgatgagaacgataagttgatcagagaggttaaggttatcaccttgaagtctaagttggtttctgatttcagaaaggatttccagttctacaaggttagagagatcaacaactaccaccacgctcacgatgcttaccttaacgctgttgttggaaccgctcttatcaagaagtacccaaagttggagtctgagttcgtttacggagattacaaggtttacgatgttagaaagatgatcgctaagtctgagcaggagatcggaaaggctaccgctaagtacttcttctactctaacatcatgaacttcttcaagaccgagatcacccttgctaacggagagatcagaaagagaccacttatcgagaccaacggagagaccggagagatcgtttgggataagggaagagatttcgctaccgttagaaaggttctttctatgccacaggttaacatcgttaagaaaaccgaggttcagaccggaggattctctaaggagtctatccttccaaagagaaactctgataagttgatcgctagaaagaaggattgggacccaaagaagtacggaggattcgattctccaaccgttgcttactctgttcttgttgttgctaaggttgagaagggaaagtctaagaagttgaagtctgttaaggagcttcttggaatcaccatcatggagcgttcttctttcgagaagaacccaatcgatttccttgaggctaagggatacaaggaggttaagaaggatcttatcatcaagttgccaaagtactctcttttcgagcttgagaacggaagaaagagaatgcttgcttctgctggagagcttcagaagggaaacgagcttgctcttccatctaagtacgttaacttcctttaccttgcttctcactacgagaagttgaagggatctccagaggataacgagcagaagcagcttttcgttgagcagcacaagcactaccttgatgagatcatcgagcaaatctctgagttctctaagagagttatccttgctgatgctaaccttgataaggttctttctgcttacaacaagcacagagataagccaatcagagagcaggctgagaacatcatccaccttttcacccttaccaaccttggtgctccagctgctttcaagtacttcgataccaccatcgatagaaaaagatacacctctaccaaggaggttcttgatgctacccttatccaccagtctatcaccggactttacgagaccagaatcgatctttctcagcttggaggagataagagaccagctgctaccaagaaggctggacaggctaagaagaagaagtgacctcaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgctcgagcaattgtacgtagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagttt

SEQ ID NO: 6 - DNA, 14601 bp, Artificial Sequence
  Synthetic construct plasmid pMBXS1126
aaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctaggcacgtgcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttctaccgattatagacgtgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataactaggttcgaattaattaatactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacaaggagcatcgtggaaaaagaagacgttccaaccacgtcttcaaagcaagtggattgatgtgatatctccactgacgtaagggatgacgcacaatcccactatccttcgccccaagcttgggcccaagcttgggtcgcgccccacggatggtataagaataaaggcattccgcgtgcaggattcacccgttcgcctctcaccttttcgctgtactctctcgccacacacaccccctctccagctccgttggagctccggacagcagcaggcgcggggcggtcacgtagtaagcagctctcggctccctctccccttgctccgtggatccatggattacaaggatgatgatgataaggattacaaggatgatgatgataagatggctccaaagaagaagagaaaggttggaatccacggagttccagctgctgataagaagtactctatcggacttgacatcggaaccaactctgttggatgggctgttatcaccgatgagtacaaggttccatctaagaagttcaaggttcttggaaacaccgatagacactctatcaagaagaaccttatcggtgctcttcttttcgattctggagagaccgctgaggctaccagattgaagagaaccgctagaagaagatacaccagaagaaagaacagaatctgctaccttcaggaaatcttctctaacgagatggctaaggttgatgattctttcttccacagacttgaggagtctttccttgttgaggaggataagaagcacgagagacacccaatcttcggaaacatcgttgatgaggttgcttaccacgagaagtacccaaccatctaccaccttagaaagaagttggttgattctaccgataaggctgatcttagacttatctaccttgctcttgctcacatgatcaagttcagaggacacttccttatcgagggagaccttaacccagataactctgatgttgataagttgttcatccagcttgttcagacctacaaccagcttttcgaggagaacccaatcaacgcttctggagttgatgctaaggctatcctttctgctagactttctaagtctcgtagacttgagaaccttatcgctcagcttccaggagagaagaagaacggacttttcggaaaccttatcgctctttctcttggacttaccccaaacttcaagtctaacttcgatcttgctgaggatgctaagttgcagctttctaaggatacctacgatgatgatcttgataaccttcttgctcagatcggagatcagtacgctgatcttttccttgctgctaagaacctttctgatgctatccttctttctgacatccttagagttaacaccgagatcaccaaggctccactttctgcttctatgatcaagagatacgatgagcaccaccaggatcttacccttttgaaggctcttgttagacagcagcttccagagaagtacaaggaaatcttcttcgatcagtctaagaacggatacgctggatacatcgatggaggagcttctcaggaggagttctacaagttcatcaagccaatccttgagaagatggatggaaccgaggagcttcttgttaagttgaacagagaggatcttcttagaaagcagagaaccttcgataacggatctatcccacaccagatccaccttggagagcttcacgctatccttcgtagacaggaggatttctacccattcttgaaggataacagagagaagatcgagaagatccttaccttcagaatcccatactacgttggaccacttgctagaggaaactctcgtttcgcttggatgaccagaaagtctgaggagaccatcaccccttggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttgttgataagggagcttctgctcagtctttcatcgagagaatgaccaacttcgataagaaccttccaaacgagaaggttcttccaaagcactctcttctttacgagtacttcaccgtttacaacgagcttaccaaggttaagtacgttaccgagggaatgagaaagccagctttcctttctggagagcagaagaaggctatcgttgatcttcttttcaagaccaacagaaaggttaccgttaagcagttgaaggaggattacttcaagaagatcgagtgcttcgattctgttgaaatctctggagttgaggatagattcaacgcttctcttggaacctaccacgatcttttgaagatcatcaaggataaggatttccttgataacgaggagaacgaggacatccttgaggacatcgttcttacccttacccttttcgaggatagagagatgatcgaggagagactcaagacctacgctcaccttttcgatgataaggttatgaagcagttgaagagaagaagatacaccggatggggtagactttctcgtaagttgatcaacggaatcagagataagcagtctggaaagaccatccttgatttcttgaagtctgatggattcgctaacagaaacttcatgcagcttatccacgatgattctcttaccttcaaggaggacatccagaaggctcaggtttctggacagggagattctcttcacgagcacatcgctaaccttgctggatctccagctatcaagaagggaatccttcagaccgttaaggttgttgatgagcttgttaaggttatgggtagacacaagccagagaacatcgttatcgagatggctagagagaaccagaccacccagaagggacagaagaactctcgtgagagaatgaagagaatcgaggagggaatcaaggagcttggatctcaaatcttgaaggagcacccagttgagaacacccagcttcagaacgagaagttgtacctttactaccttcagaacggaagagatatgtacgttgatcaggagcttgacatcaacagactttctgattacgatgttgatcacatcgttccacagtctttcttgaaggatgattctatcgataacaaggttcttacccgttctgataagaacagaggaaagtctgataacgttccatctgaggaggttgttaagaagatgaagaactactggagacagcttcttaacgctaagttgatcacccagagaaagttcgataaccttaccaaggctgagagaggaggactttctgagcttgataaggctggattcatcaagagacagcttgttgagaccagacagatcaccaagcacgttgctcagatccttgattctcgtatgaacaccaagtacgatgagaacgataagttgatcagagaggttaaggttatcaccttgaagtctaagttggtttctgatttcagaaaggatttccagttctacaaggttagagagatcaacaactaccaccacgctcacgatgcttaccttaacgctgttgttggaaccgctcttatcaagaagtacccaaagttggagtctgagttcgtttacggagattacaaggtttacgatgttagaaagatgatcgctaagtctgagcaggagatcggaaaggctaccgctaagtacttcttctactctaacatcatgaacttcttcaagaccgagatcacccttgctaacggagagatcagaaagagaccacttatcgagaccaacggagagaccggagagatcgtttgggataagggaagagatttcgctaccgttagaaaggttctttctatgccacaggttaacatcgttaagaaaaccgaggttcagaccggaggattctctaaggagtctatccttccaaagagaaactctgataagttgatcgctagaaagaaggattgggacccaaagaagtacggaggattcgattctccaaccgttgcttactctgttcttgttgttgctaaggttgagaagggaaagtctaagaagttgaagtctgttaaggagcttcttggaatcaccatcatggagcgttcttctttcgagaagaacccaatcgatttccttgaggctaagggatacaaggaggttaagaaggatcttatcatcaagttgccaaagtactctcttttcgagcttgagaacggaagaaagagaatgcttgcttctgctggagagcttcagaagggaaacgagcttgctcttccatctaagtacgttaacttcctttaccttgcttctcactacgagaagttgaagggatctccagaggataacgagcagaagcagcttttcgttgagcagcacaagcactaccttgatgagatcatcgagcaaatctctgagttctctaagagagttatccttgctgatgctaaccttgataaggttctttctgcttacaacaagcacagagataagccaatcagagagcaggctgagaacatcatccaccttttcacccttaccaaccttggtgctccagctgctttcaagtacttcgataccaccatcgatagaaaaagatacacctctaccaaggaggttcttgatgctacccttatccaccagtctatcaccggactttacgagaccagaatcgatctttctcagcttggaggagataagagaccagctgctaccaagaaggctggacaggctaagaagaagaagtgacctcaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgctcgagcaattgtacgtagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagttt

SEQ ID NO: 7 - DNA, 2433 bp, Camelina sativa
atggatataagcaatgaagcaggcgttgatgcgttctcaattataggacccacgtctataatcggtagaacaattgcctaccggatcttgttctgcaactcagtgtctatattcagacacaaactctacagatttctcaagtttttcttcagaggaggcagggttttactatcaccgtttgtgtccttactacatcccaggaatccacaagggatattagcaatggtgatgacgatggcctttctgttgaatcgctacacaagcctaaaagcaaaagctgagatggcttacaggaggaaattctggaggaacatgatgagagctgcattgacatacgaggaatggtctcacgccgcaaagatgctggataaagagactccaaaggtgaacgaggcagatctcttcgatgtggagctcgtgaggaataagcttgaggagcttaagcatagacgtcacgagggatctctaaaagacattatcttttgtatgagagctgatcttgttagaaaccttggtaacatgtgtaaccctgagcttcacaagggaaggcttcatgtgccgagattaattaaagagtacatcgatgaggtttctacacagctcaggatggtttgtgacacggacactgaagagcttcctttggaggagaaactctcttttatgcatgagactagacacgcgtatggaagaacggctctgcttctgagcggaggagcttctctaggggctttccatcttggtgtggtcaagacgcttgtggaacataagctcttgccgagaattatagccggttcaagcgtgggatccgtaatgtgtgctgttgtggggacaaggtcatggcctgagttacagagcttctttgaaggctcttggcatgctttgcagttctttgatcaaatgggagggattttcactactgtgaaacgggttatgactcaaggggcagtccatgaaatccggcatttgcaatggaagttgaggaatctcaccaacaaccttacattccaagaagcttacgacataacaggacggattctagggataacagtttgttccttgaggaaacacgaaccgcctagatgtctcaattatctgacttcgcctcacgttgtgatatggagtgcagtgactgcatcttgtgctttcccaggtcttttcgaggctcaagaactcatggccaaagatagaactggagagatcgtaccttatcacccaccttttaacttagatcctgaagagggctcaggagaatcagttcggcgctggagggacggtagtttggagatggacttaccgatgatgcaactcaaagagcttttcaatgtcaaccatttcattgtcagtcaagccaaccctcacatagcacccttcttgaggatgaaggagtttgttagagcttgtggaggtcgatttgctgcaaagctcgctcaactagctgagatggaagtgaagcatagatgtcatcaagtactagaactcgggcttcctctaagagaagtagcttccctatttgctcaagaatgggaaggtgatgtcactatcgtcatgccagctactttttctcagtacttaaagatcatacagaacccaagcaatgtagagattcaaaaggcggcaaatcaaggaaggagatgcacctgggagaaactcgcagtcatcaaagcaaacttcgggatcgaactagcactcgacgagtgcgtcgccgttctcaaccacatgcgccgccttaaacgcagcgctgaaagagccgctgctttctccgcaatctcttcctccccaccatccaaacatctcttagccggagccaacagattcaacgcatctaaacgaatcccttcctggaactgcatagctcgtcagaactcatccggatccgtcgacgatgatgtgctcgcagaagcttcacggttgtaccagcacatcgtggttggatccgggaagactagtaaccgaaccagcaacttaagccattcgtatgacgcaggaagcgaaggagagtcaccagaagctgaagattggacgagatctggtggaccgttgatgaggacatcgtcagctcagatgttcacagactacgtccagaatctcaacgccgttgatccagaacagacgacgattggtgactccgatagggctccggagaccgaattgaacgcggctgctgctgctgcttcttcttcttcttcttccagcatcacggttacagaaggagattatcttcagacggggaggacacacaacggaatcgtgttgaatctcgttagaggagagaatctgaggatgaatcaggatctggaagatagcgaaactgaatgtgaaactccggagagcgtgcaactcgaatcgccggaaaaggacattattgacggaaatagctcggcgtctgaagacggagacactagtactcctcaggcgaatctaacccacgagtga

SEQ ID NO: 8 - DNA, 2538 bp, Camelina sativa
atggatataagcaatgaagcaggcgttgatgcgttctcaattataggacccacgtctataatcggtagaacaattgcctaccggatcttgttctgcaactcagtgtctatattcagacacaaactctacagatttctcaagtttttcctcagaggaggcagggttttactatcaccgtttgtgtccttactacatcccaggaatccacaagggatattagcaatggtgatgacgatggcctttctgttgaatcgctacacaagcctaaaagcaaaagctgagatggcttacaggaggaaattctggaggaacatgatgagagctgcattgacatacgaggaatggtctcacgccgcaaagatgcttgataaagagactccaaaggtgaacgaggcagatctcttcgatgtggagctcgtaaggaataagcttgaggagcttaagcatagacgtcacgagggatctctaaaagacattatcttctgtatgagagctgatcttgttagaaacctcggtaacatgtgtaaccctgagcttcacaagggaaggcttcatgtgccgagattaattaaagagtacatcgatgaggtttctacacagctcaggatggtttgtgacacggacactgaagagcttcctttggaggagaaactctcttttatgcatgagactagacacgcgtatggaagaacggctctgcttctgagcggaggagcttctctaggagctttccatcttggtgtggtcaagacgcttgtagaacataagctcttgccgagaattatagccggttcaagcgtgggatccgtaatgtgtgcggttgtggggacaaggtcatggcctgagttacagagcttctttgaaggctcttggcatgctttgcagttctttgatcagatgggagggattttcactactgtgaaacgggttatgactcaaggcgcagtccatgagatccggcatttgcaatggaagttgaggaatctcaccaacaaccttacattccaagaagcttacgacataacaggacggattctagggataacagtttgttccttgaggaaacacgaaccccctagatgtctcaattatctgacgtcgcctcatgttgtgatatggagtgcagtgactgcatcttgtgctttcccaggtctttttgaggctcaagaactcatggccaaagatagaactggagagattgttccttatcacccaccttttaacttagatcctgaagagggctcaggagaatcagtacggcgctggagggacggtagtttggagatggacttaccgatgatgcaactgaaagagcttttcaatgtcaaccatttcattgtcagtcaagccaaccctcacatagcacccttcttgaggatgaaggagtttgttagagcttgtggaggtcgatttgctgcaaagctcgctcaactcgcggagatggaagtgaagcatagatgtcatcaagtactagaactcgggcttcctctaagagaagtagcttccctatttgctcaagaatgggaaggtgatgtcactatcgtcatgccagctactttttctcaggtaaccactcactgcaaacaatgtatcccaactagaacatttcactctaattcaattcttgtattgagtctctctatatgctttgctctgtgtgtttccggattccagtacttaaagatcatacagaacccaagcaatgtagagattcaaaaggcggcaaatcaagggaggagatgcacctgggagaaactcgcagtcatcaaagcaaacttcgggatcgaactagcactcgacgagtgcgtcgccgttctcaaccacatgcgccgccttaaacgcagcgctgaaagagccgctgctttctcagccatctcttcctccccaccacccaaacatctcttggccggagccaacagattcaacgcctctaaacgaatcccttcctggaactgcatagctcgtcaaaactcatccggatccgtcgacgatgatgtgctcgcagaagcttcacggttgtaccagaacatcgtggttggatccgggaagactagtaaccgaaccagcaacttaagccattcgtatgacgcaggaagcgaaggagaatcaccagaagctgaagactggacgagatctggtggaccgttgatgaggacatcgtcagctcagatgttcactgcctacgtccaaaatctcaacgccgctgatcctgaacagacgacgattggtgactccgatagggctccggagaccgaattgaacgccgctgctgctgcttcttcttcttcttcttccagcataacggttacagaaggcgattatcttcagacggggaggacacataacggaatcgtgttgaatctcgtcagaggagagaatctgaggatgaatcaggatctggaagatagcgaaaacgaatgtgaaactccggagagcgtgcaactcgaatctccggagaaggacattattgacggaaatagctcggcgtctgaagacggagacactagtactgctcaggcgaatctaacccacgagtga

SEQ ID NO: 9 - DNA, 2421 bp, Camelina sativa
atggatataagcaatgaagcaggcgttgatgcgttctcaattataggacccacgtctataatcggtagaacaattgcctaccggatcttgttctgcaactcagtgtctatattcagacacaaactctacagatttctcaagttttttctcagaggaggcagggttttactatcaccgtttgtgtccttactacatcccaggaatccacaagggatattagcaatggtgatgacgatggcttttctgttgaatcgctacacaagcctaaaagcaaaggctgagatggcttacaggagaaaattctggaggaacatgatgagagctgcattgacatacgaggaatggtctcacgccgcaaagatgcttgataaagagactccgaaggtgaacgagtcagatctcttcgatgtggagctcgtgaggaataagcttgaggagcttaagcataggcgtcacgagggatctctaaaagatattatcttctgtatgagagctgatcttgttaggaatctcggtaacatgtgtaaccctgagcttcacaaggaaaggcttcatgtgccgagattaattaaagagtacatcgatgaggtttctacacagctcaggatggtatgtgacacggacactgaagagcttcctttggaggagaaactctcttttatgcatgaaactagacacgcgtatggcagaacggctctgcttctgagcggaggagcttctctaggggctttccatcttggtgtggtgaagacgcttgtggaacataagctcttgccgagaattatagccggatcaagcgtgggatccgtaatgtgtgctgttgtggggacaaggtcatggcctgagttacagagcttctttgaaggctcttggcatgctttgcagttctttgatcagatgggagggattttcactactgtgaaacgggttatgactcaaggcgcagtgcacgagatccggcatttgcaatggaagttgaggaatctcaccaacaaccttacattccaagaagcttacgacataacaggacggattttagggataacagtttgttccttgaggaaacacgaaccgcctagatgtctcaattatctgacttctcctcatgttgtaatatggagtgcagtgactgcatcttgtgctttcccaggtctttttgaggctcaagaactcatggccaaagatagaactggagagattgttccttatcacccaccttttaacttagatcctgaagaaggctcaggagaatcacttcggcgctggagggacggtagtttggagatggacttaccgatgatgcaactcaaagagcttttcaatgtcaaccatttcattgtcagtcaagccaaccctcacatagcacccttcttgaggatgaaagagtttgttagagcttgtggaggtcgatttgctgcaaagctcgctcaactagcagagatggaagtgaagcatagatgtcatcaagtactagaactcgggcttcctctaagagaagtagcttccctatttgctcaagaatgggaaggtgatgtcactatcgtcatgccagctactttttctcagtacttaaagatcatacagaaccctagcaatgtagagattcaaaaggcggcaaatcaaggaaggagatgcacctgggagaaactagcagtcatcaaagcaaacttcgggatcgaactagcactcgacgagtgcgtcgccgttcttaaccacatgcgccgccttaaacgcagcgctgaaagagccgctgctttctcagccatctcttcctccccaccatctaaacatctcttagccggagccaacagattcaacgcctctaaacgaatcccttcctggaactgcatagctcgtcagaactcatccggatccgtcgacgatgatgtgctcgccgaagcttcacggttataccagcacatcgtggttggatccgggaagcctagtaatcgaaccagcaacttaagccacgcaggaagcgaagtagaatctccagaagctgaagactggacgagatctggtggaccgttgatgaggacgtcgtcagcccagatgttcactgactacgtccagaatctcaacgccgccgctgatcctgaacagacgacgattggaggtgactccgatagagctccggaggtagaattgaacgcggctgctgcttctccttcttccagcatcacggttacagaaggagattatcttcagacggggaggacacataacggattcgtgttgaatcttgttagaggagagaatctgaggatgaatcaggatctggaagatagcgaaaacgaatgtgaaactccggagagcgtgcagctcgaatcgccggaaaaggatattattgacggaaatagctcggcgtctgaagacggagacactagtactactgctcaggcgaatctaacccacgagtga

SEQ ID NO: 10 - DNA, 20 bp, Artificial Sequence
  Synthetic construct guide sequence SDP1-like #4
gttctaccgattatagacgt

SEQ ID NO: 11 - DNA, 822 bp, Camelina sativa
atgggaaagagagcgactaatgctctgaagagagaggagttaaacagaggagcttggactgatcatgaagacaagacccttagagattacatcacgactcatggtgaaggcaagtggagcgttctcccaaaacaagctgggctcaagagatgtggcaaaagctgtagactccgttggaagaactacttaagacccgggataaagcgcggaaacatctcatctgatgaagaagaactcataatccgtctccataatctccttggaaacagatggtcgttgatagctgggaggcttccaggccgaacagacaatgaaataaagaaccattggaactcgaacctccgcaaaagacttcccaaaactcaaaccaacccaccgaaacgtataaaacattcgaccaagaacgagaataataagtcacaaactatagtctcaaaatgcatgttttcttgtagtgttatacgtacaaaggcgatgaggtgctcgaaggctcttctcgtctccgatctatctcttcagaagcagagtagtatcagtccacttcctttgaaaaaacaagagatgatggatcaaggtgggacgtcatcttccttgatgggagatctcgagttcgatttcgataggatccagtcggagtttttattcccggatttgatcgattttgatggtttggactgtggaaacgtaacatctcttgtttcgtctgaggacattttgggtaatttggttccagctcaaggtaatctcgatctcaatagacctttcacttcttatcatcaccatggcgacgatgaagactggctcggagacttcaactgttga

SEQ ID NO: 12 - DNA, 786 bp, Camelina sativa
atgggaaagagaacgactaatgctctgaagagagaggagttaaacagaggagcttggactgatcatgaagacaagacccttagagattacatcacgactcatggtgaaggcaagtggagcactctcccaaaccaagctgggctcaagagatgtggcaaaagctgtagactccggtggaagaactacttaagaccggggataaagcgcgggaacatcgcacctgatgaagaagaactcataatccgtctccataatctccttggaaacagatggtcgttgatagctgggaggcttccaggccgaacagacaatgaaataaagaaccattggaactcaaacctccgcaaaagacttcccaaaactcaaaccaacccaccgaaacgtataaaacattcgaccaagaacgagaacaatgtccatgttatacgtacaaaggcgatgaggtgctcgaaggctcttctcctctcggatctgtctcttcagaagcagagtagtatcagtccacttcctttgaaagaacaagagatgatgggtcaaggtgggacgtcatcttcgttgatgggagatctcgagttcgatttcgataggatccagtcggagtttttattcccggatttgattgattttgatggtttggactgtggaaacgtaacatctcttgtttcgtctgaggagattttgggagatttggttccagctgaaggtaatctcgatctcaatagacctttcacttcttatcaccaccatggcgacgatgaagactggctcggaaacttcaactgttga

SEQ ID NO: 13 - DNA, 786 bp, Camelina sativa
atgggaaagagagcgactaatgctctgaagagagaggagttaaacagaggagcttggactgatcatgaagacaagaccctaagagattacatcacgactcatggtgaaggcaagtggagtactctcccaaacaaggctgggctcaagagatgtggcaaaagctgtagactccggtggaagaactacttaagacccgggataaagcgcggaaacatctcacctgatgaagaagaactcataatccgtctccataatctccttggaaacagatggtcgttgatagctgggaggcttccaggccgaacagacaatgaaataaagaaccattggaactcaaacctccgcaaaagacttcccaaaactcaaacaaacccaccgaaacgtataaaacattcgaccaagaacgagaacaatgtccgtgttatacgtacaaaggcgatgaggtgctcgaaggctcttctcctctcggatctatctcttcagaagcagagtagtatcagtccacttcctttgaaagaacaagagatgatggatcaaggtgggacgtcatcttcgttgatgggagatcttcagttcgatttcgataggatccagtcggagtttttattcccggatttgattgattttgatagtttggactgtggaaacgtaacatcttttgtttcgtctgaggagattttgggagatttggttccagctcaaggtaatctcgatctcaatagacctttcacttcttatcatcaccatggcgacgatgaagattggctcggaaacttcaactgttga

SEQ ID NO: 14 - DNA, 20 bp, Artificial Sequence
  Synthetic construct guide sequence SDP1 #77
caagaaagcatgaacctcct

SEQ ID NO: 15 - DNA, 20 bp, Artificial Sequence
  Synthetic construct guide sequence TT2 #106/107
gatggtcgttgatagctggg

SEQ ID NO: 16 - DNA, 16091 bp, Artificial Sequence
  Synthetic construct plasimd MBXS1140
cgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgatggtcgttgatagctggggttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataactaggcacgtgcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttctaccgattatagacgtgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaataactaggcacgtgcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattcaagaaagcatgaacctcctgttttagagctagaaatagcaagttaaaataaggctagtccgttatcaacttgaaaaagtggcaccgagtcggtgcttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatagacgtctctagaactaggttcgaattaattaatactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacaaggagcatcgtggaaaaagaagacgttccaaccacgtcttcaaagcaagtggattgatgtgatatctccactgacgtaagggatgacgcacaatcccactatccttcgccccaagcttgggcccaagcttgggtcgcgccccacggatggtataagaataaaggcattccgcgtgcaggattcacccgttcgcctctcaccttttcgctgtactctctcgccacacacaccccctctccagctccgttggagctccggacagcagcaggcgcggggcggtcacgtagtaagcagctctcggctccctctccccttgctccgtggatccatggattacaaggatgatgatgataaggattacaaggatgatgatgataagatggctccaaagaagaagagaaaggttggaatccacggagttccagctgctgataagaagtactctatcggacttgacatcggaaccaactctgttggatgggctgttatcaccgatgagtacaaggttccatctaagaagttcaaggttcttggaaacaccgatagacactctatcaagaagaaccttatcggtgctcttcttttcgattctggagagaccgctgaggctaccagattgaagagaaccgctagaagaagatacaccagaagaaagaacagaatctgctaccttcaggaaatcttctctaacgagatggctaaggttgatgattctttcttccacagacttgaggagtctttccttgttgaggaggataagaagcacgagagacacccaatcttcggaaacatcgttgatgaggttgcttaccacgagaagtacccaaccatctaccaccttagaaagaagttggttgattctaccgataaggctgatcttagacttatctaccttgctcttgctcacatgatcaagttcagaggacacttccttatcgagggagaccttaacccagataactctgatgttgataagttgttcatccagcttgttcagacctacaaccagcttttcgaggagaacccaatcaacgcttctggagttgatgctaaggctatcctttctgctagactttctaagtctcgtagacttgagaaccttatcgctcagcttccaggagagaagaagaacggacttttcggaaaccttatcgctctttctcttggacttaccccaaacttcaagtctaacttcgatcttgctgaggatgctaagttgcagctttctaaggatacctacgatgatgatcttgataaccttcttgctcagatcggagatcagtacgctgatcttttccttgctgctaagaacctttctgatgctatccttctttctgacatccttagagttaacaccgagatcaccaaggctccactttctgcttctatgatcaagagatacgatgagcaccaccaggatcttacccttttgaaggctcttgttagacagcagcttccagagaagtacaaggaaatcttcttcgatcagtctaagaacggatacgctggatacatcgatggaggagcttctcaggaggagttctacaagttcatcaagccaatccttgagaagatggatggaaccgaggagcttcttgttaagttgaacagagaggatcttcttagaaagcagagaaccttcgataacggatctatcccacaccagatccaccttggagagcttcacgctatccttcgtagacaggaggatttctacccattcttgaaggataacagagagaagatcgagaagatccttaccttcagaatcccatactacgttggaccacttgctagaggaaactctcgtttcgcttggatgaccagaaagtctgaggagaccatcaccccttggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttgttgataagggagcttctgctcagtctttcatcgagagaatgaccaacttcgataagaaccttccaaacgagaaggttcttccaaagcactctcttctttacgagtacttcaccgtttacaacgagcttaccaaggttaagtacgttaccgagggaatgagaaagccagctttcctttctggagagcagaagaaggctatcgttgatcttcttttcaagaccaacagaaaggttaccgttaagcagttgaaggaggattacttcaagaagatcgagtgcttcgattctgttgaaatctctggagttgaggatagattcaacgcttctcttggaacctaccacgatcttttgaagatcatcaaggataaggatttccttgataacgaggagaacgaggacatccttgaggacatcgttcttacccttacccttttcgaggatagagagatgatcgaggagagactcaagacctacgctcaccttttcgatgataaggttatgaagcagttgaagagaagaagatacaccggatggggtagactttctcgtaagttgatcaacggaatcagagataagcagtctggaaagaccatccttgatttcttgaagtctgatggattcgctaacagaaacttcatgcagcttatccacgatgattctcttaccttcaaggaggacatccagaaggctcaggtttctggacagggagattctcttcacgagcacatcgctaaccttgctggatctccagctatcaagaagggaatccttcagaccgttaaggttgttgatgagcttgttaaggttatgggtagacacaagccagagaacatcgttatcgagatggctagagagaaccagaccacccagaagggacagaagaactctcgtgagagaatgaagagaatcgaggagggaatcaaggagcttggatctcaaatcttgaaggagcacccagttgagaacacccagcttcagaacgagaagttgtacctttactaccttcagaacggaagagatatgtacgttgatcaggagcttgacatcaacagactttctgattacgatgttgatcacatcgttccacagtctttcttgaaggatgattctatcgataacaaggttcttacccgttctgataagaacagaggaaagtctgataacgttccatctgaggaggttgttaagaagatgaagaactactggagacagcttcttaacgctaagttgatcacccagagaaagttcgataaccttaccaaggctgagagaggaggactttctgagcttgataaggctggattcatcaagagacagcttgttgagaccagacagatcaccaagcacgttgctcagatccttgattctcgtatgaacaccaagtacgatgagaacgataagttgatcagagaggttaaggttatcaccttgaagtctaagttggtttctgatttcagaaaggatttccagttctacaaggttagagagatcaacaactaccaccacgctcacgatgcttaccttaacgctgttgttggaaccgctcttatcaagaagtacccaaagttggagtctgagttcgtttacggagattacaaggtttacgatgttagaaagatgatcgctaagtctgagcaggagatcggaaaggctaccgctaagtacttcttctactctaacatcatgaacttcttcaagaccgagatcacccttgctaacggagagatcagaaagagaccacttatcgagaccaacggagagaccggagagatcgtttgggataagggaagagatttcgctaccgttagaaaggttctttctatgccacaggttaacatcgttaagaaaaccgaggttcagaccggaggattctctaaggagtctatccttccaaagagaaactctgataagttgatcgctagaaagaaggattgggacccaaagaagtacggaggattcgattctccaaccgttgcttactctgttcttgttgttgctaaggttgagaagggaaagtctaagaagttgaagtctgttaaggagcttcttggaatcaccatcatggagcgttcttctttcgagaagaacccaatcgatttccttgaggctaagggatacaaggaggttaagaaggatcttatcatcaagttgccaaagtactctcttttcgagcttgagaacggaagaaagagaatgcttgcttctgctggagagcttcagaagggaaacgagcttgctcttccatctaagtacgttaacttcctttaccttgcttctcactacgagaagttgaagggatctccagaggataacgagcagaagcagcttttcgttgagcagcacaagcactaccttgatgagatcatcgagcaaatctctgagttctctaagagagttatccttgctgatgctaaccttgataaggttctttctgcttacaacaagcacagagataagccaatcagagagcaggctgagaacatcatccaccttttcacccttaccaaccttggtgctccagctgctttcaagtacttcgataccaccatcgatagaaaaagatacacctctaccaaggaggttcttgatgctacccttatccaccagtctatcaccggactttacgagaccagaatcgatctttctcagcttggaggagataagagaccagctgctaccaagaaggctggacaggctaagaagaagaagtgacctcaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgctcgagcaattgtacgtagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagaaaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctggtaccaatgagctctgtccaacagtctcagggttaactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcgcgcccctagg

SEQ ID NO: 17 - protein, 358 aa, Chlamydomonas reinhardtii
MSSDAMTINESLMEVEHTPAVHKRILDILPGISGGVARVMIGQPFDTIKVRLQVLGQGTALAAKLPPSEVYKDSMDCIRKMIKSEGPLSFYKGTVAPLVGNMVLLGIHFPVFSAVRKQLEGDDHYSNFSHANVLLSGAAAGAAGSLISAPVELVRTKMQMQRRAALAGTVAAGAAASAGAEEFYKGSLDCFKQVMSKHGIKGLYRGFTSTILRDMQGYAWFFLGYEATVNHFLQNAGPGVHTKADLNYLQVMAAGVVAGFGLWGSMFPIDTIKSKLQADSFAKPQYSSTMDCLKKVLASEGQAGLWRGFSAAMYRAIPVNAGIFLAVEGTRQGIKWYEENVEHIYGGVIGPATPTAAQ

SEQ ID NO: 18 - DNA, 11136 bp, Artificial Sequence
  Synthetic construct plasmid pMBXO58
tggcaggatatattgtggtgtaaacaaattgacgcttagacaacttaataacacattgcggacgtttttaatgtactgaattaacgccgaattaattcgagctcggatctgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaagattctgcagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatcgattcgatttggtgtatcgagattggttatgaaattcagatgctagtgtaatgtattggtaatttgggaagatataataggaagcaaggctatttatccatttctgaaaaggcgaaatggcgtcaccgcgagcgtcacgcgcattccgttcttgctgtaaagcgttgtttggtacacttttgactagcgaggcttggcgtgtcagcgtatctattcaaaagtcgttaatggctgcggatcaagaaaaagttggaatagaaacagaatacccgcgaaattcaggcccggttgccatgtcctacacgccgaaataaacgaccaaattagtagaaaaataaaaactgactcggatacttacgtcacgtcttgcgcactgatttgaaaaatctcagaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagatcacaagtttgtacaaaaaagcaggctccgcggccgcccccttcaccatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaagaacagaaactgatctctgaagaagatctgtgaaagggtgggcgcgccgacccagctttcttgtacaaagtggtgcctaggtgagtctagagagttaattaagacccgggactagtccctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 19 - DNA, 11380 bp, Artificial Sequence
  Synthetic construct plasmid pMBXO84
tcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcaccgaagaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggaacacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgtacgtagtgtttatctttgttgcttttctgaacaatttatttactatgtaaatatattatcaatgtttaatctattttaatttgcacatgaattttcattttatttttactttacaaaacaaataaatatatatgcaaaaaaatttacaaacgatgcacgggttacaaactaatttcattaaatgctaatgcagattttgtgaagtaaaactccaattatgatgaaaaataccaccaacaccacctgcgaaactgtatcccaactgtccttaataaaaatgttaaaaagtatattattctcatttgtctgtcataatttatgtaccccactttaatttttctgatgtactaaaccgagggcaaactgaaacctgttcctcatgcaaagcccctactcaccatgtatcatgtacgtgtcatcacccaacaactccacttttgctatataacaacacccccgtcacactctccctctctaacacacaccccactaacaattccttcacttgcagcactgttgcatcatcatcttcattgcaaaaccctaaacttcaccttcaaccgcggccgcttcgaaaaaatgtctagtgatgccatgaccatcaatgagtctcttatggaagtcgaacatactccagctgtgcataaaaggattcttgacattttaccgggtatcagtggcggggttgccagagttatgataggtcagcccttcgacacaatcaaagtgcgtctacaagtgttggggcagggtacggctctcgctgccaaacttcctcctagtgaagtttacaaggacagcatggattgcattcgtaagatgattaagtcggagggtccactaagcttttacaagggaacagttgccccactcgtcggaaacatggtattgcttggcatccattttccggtcttttccgcggttagaaagcagttggagggtgatgatcattactctaacttttcacacgccaatgtactgcttagcggcgctgcggcaggagctgcgggatcactcatttcggctcctgttgaactggttagaacgaaaatgcaaatgcaaaggcgagccgcacttgcgggtacagtggctgctggtgcagctgcatctgctggagctgaggagttctataagggaagtcttgattgtttcaaacaagttatgtctaagcatgggattaaaggattgtataggggttttacttcaactatactacgagatatgcagggttatgcttggttcttcctcggatatgaggcgactgtcaatcacttcttgcaaaatgcgggaccaggtgttcataccaaggctgacttgaattaccttcaagtgatggccgctggggttgttgctggatttggattatggggctccatgtttccaatcgataccatcaaatctaaactccaagccgatagctttgccaaacctcaatattcatccacaatggattgtcttaagaaagtattagcaagtgagggacaggccggcttgtggagagggttcagcgcagcaatgtatagagcaataccggtgaacgctggcattttcctcgctgttgaagggacacgtcagggtataaagtggtacgaggaaaacgtggaacacatctacggaggtgtcattggtcccgctacgcctactgcagcacaatgacgaaatttaaatgcggccgctgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaacctcagcactagtcgtcaaagggcgacaccccctaattagcccaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgac

SEQ ID NO: 20 - DNA, 708 bp, Camelina sativa
atgagaatcagctgcaacgggtgtagggtcctacgcaaaggttgcaacaaggattgcaccataagaccatgtcttcaatggatcaaatccgcagactcccaagctaatgctacactcttcctcgccaagttctacggtcgagccggtcttcttaacctcatcgaatccggtcctgatcaccttcgtcccgcaatatttaggtcacttctgtacgaggcgtgtggtcggatcgtgaatcctgtggatggttcggtgggtttgatgtggtcagggaactgggcccaatgtcaggcagccgttgacgcggtgctcaacggcttacccataactcacacacctcttccaagtgcatctgcgtctcatcagatcattcctcctcacaggacttatgacatacgccacgtggctaaagatccaacaaccggcggcgacagttcggagagtctggccacacgtgttaacgctaacaagggcaagacacaaactggccgattcaaacgccatgctgagtccgtgaaccaacaaggcgaatgtagtcatgacacatggcaactcccaagttctggtgctacgcatggctatggtcacttcacgttggagactgtggagagtcgaagggaagctccacttgatcaaagttcttcgaatcttgggtttgatgatcaagtcgatatcaacgaggttggcttagagctcagacttggttag

SEQ ID NO: 21 - protein, 235 aa, Camelina sativa
MRISCNGCRVLRKGCNKDCTIRPCLQWIKSADSQANATLFLAKFYGRAGLLNLIESGPDHLRPAIFRSLLYEACGRIVNPVDGSVGLMWSGNWAQCQAAVDAVLNGLPITHTPLPSASASHQIIPPHRTYDIRHVAKDPTTGGDSSESLATRVNANKGKTQTGRFKRHAESVNQQGECSHDTWQLPSSGATHGYGHFTLETVESRREAPLDQSSSNLGFDDQVDINEVGLELRLG

SEQ ID NO: 22 - DNA, 747 bp, Camelina sativa
atgaaaaaaatgttgaaaagtgtaagtccaatggcattctacgatataggtgaccagcaatactctgctttagggtacatcttaagcaagcctgggaatgtaggagcgtacgacatagaccctccgatgccaaccatcgacgatgcgatctacggctcagatgagtttcgcatgtacgcgtacaagatcaaacggtgtcctcgtacccgcagccacgactggactgagtgtccctacgctcaccgcggcgagaaagccacacgccgtgatcctcggcgtcacacttactgcgcagtcgcatgcccggctttcagaaacggagcatgccaccgtggggactcatgcgaattcgcacacggtgtattcgagtactggctccacccggcgcgttacagaacacgcgcttgcaacgccgggaacttgtgtcagaggaaagtgtgtttcttcgcacacgcgccggagcagcttcggcagtctgagggaaagcataggtgcaggtatgcttacaggccagtgagggctagaggcggaaacagtaatggggtggcgatgagattggacggcgaggattacgacacgtcacgatctccggagaaaagcgggaaaagtggtttagatggtaacgaggagaaagtgttgttgaagtgttggagtcggatgagcattgtggatgatcattatgagctgtctgatttggatttcgatttgaatttcggtttggattggatctcagagctggtggattag

SEQ ID NO: 23 - protein, 248 aa, Camelina sativa
MKKMLKSVSPMAFYDIGDQQYSALGYILSKPGNVGAYDIDPPMPTIDDAIYGSDEFRMYAYKIKRCPRTRSHDWTECPYAHRGEKATRRDPRRHTYCAVACPAFRNGACHRGDSCEFAHGVFEYWLHPARYRTRACNAGNLCQRKVCFFAHAPEQLRQSEGKHRCRYAYRPVRARGGNSNGVAMRLDGEDYDTSRSPEKSGKSGLDGNEEKVLLKCWSRMSIVDDHYELSDLDFDLNFGLDWISELVD

SEQ ID NO: 24 - DNA, 636 bp, Camelina sativa
atggataacgtgagtgttttggcaaagggagagaaccaactgaacggagagaagcccccaccggaagtgtgtgcaaggtgtagctctaacaacacaaagttctgctactataacaactatagcatgtctcagccgcgctacttctgcaaggaatgtcgtcgatactggactcaaggtgggactttgaggaacgtcccggttggtggaggttgccgtaagcccaagcgtccaaggatagaacactcctctgtttctcaggtggcttcctttgagaaccaaccagttaatcatcaacctttcagacttgttcatgaaaacaacgagtctgttggactttttggtggttctacttcttcttcctcttctcctgctgctgttgctgccgttcttgggaagcatttgggttatttggctgagtttcgtggtgtaaccaatgtgcctcaagttcgaggtcttcgacctgtactgctggatcgcttggattttgtgcatcagcacaagtggaaccaaagctttgacaacactatgaatgtgaatcatgaagcaagctctagtggaagcagaggatcttccgacaccgagacgaacagcaacaacaacaacaacaacaaagatcagaagcacctgtttgattag

SEQ ID NO: 25 - protein, 211 aa, Camelina sativa
MDNVSVLAKGENQLNGEKPPPEVCARCSSNNTKFCYYNNYSMSQPRYFCKECRRYWTQGGTLRNVPVGGGCRKPKRPRIEHSSVSQVASFENQPVNHQPFRLVHENNESVGLFGGSTSSSSSPAAVAAVLGKHLGYLAEFRGVTNVPQVRGLRPVLLDRLDFVHQHKWNQSFDNTMNVNHEASSSGSRGSSDTETNSNNNNNNKDQKHLFD

SEQ ID NO: 26 - DNA, 1881 bp, Camelina sativa
atgacttttgctgaatctctccacaacaaaagttctgtcttgagtatcaacatcctagttgtggatgatgatcctgttatccttgaagttcttccaaaaatgcttgtaaagtcgaaatacagagatccatcggttatggagatcaaagttatagctgaagaagacccggtgaaagcattggctattctaaaacttcaacgaaacaatatcgatctcataatcacagattattacatgcctagcatgaacggtctacaactcaaaagacgaatcactgaggaaattggaaatttacctgtcattgttgtatcagcggacgccaacaaggagcaggagagtttaacttgtggagcgctggggttccttccaaaacctatacaagcaactgacctaccaaagatataccaacttgctttaacctgcaagaggaatggtaagtccaactacaaggccgacgacacaggtgctagtgtcccccagcaaatcaagatgcttcctgaacaagctaatgtcttgaagaccaggaagaagtactcatctaaagctgcttccagatccttaaacagtacaaacggaagtggtctaattagtaccgatggttcaagaaaaaatcggaaaagaaaacctaacggtggtagtgaggatggtgctccgtcgcagccatccaagaagactaagatttcgtggacggatggtcttaacgacctattcctacaagctatccgacatattggtcttgataaggctgtgccaaaaaagatcttagcgttcatgaacgtaccatacttgacaagagagaacgttgccagccatttacagaaatatcggatattcttgaggaaagtggcggatcaaggtttgatgagcattatgtcagctagaggcatagactcgatgtttcgacatactcaaatcaaggagtcctacttcaaccgatacacaccctctacttctctgtacgacacaagtcttaacaatagatcaatctattccaaacccggacaaggctttggacagtcgagacttttgtcgaccacacgtgagccacccgtccgtttcaaccagatgccttacaactacatgaaccggtcatctacttacgagacgcaccatgttggttctggatcaaacttgacgctacccatccaaagcaacctcagtctcgccaaccaaccatttcaaaacgaggaattaagaagctttttcaaaccacctgtgatggcgaacaaaatcggccaaacatctcaagttcatgggtttggacaacttggaccgttagctattaatttcaacaataacatgatgagtagctttggaagttttactccaaatcaaccaggacttggccacttctcgtatgggatgcaatcgttcttaaacaatgagaacactgcatataaccctgagccacatgccaatgcaactacacagccaaatcttgaacttcctcaactggaggatctcaacatacacagtggtctcggcaagactaatgagcttccttacaacaccagcaactttcaatccgatcacaacaagcagcaacaaggagaagaggcttccaacaaatttgagctcccatcatttttttcggctgaactgaatcagtttttatctcttgaagacgatagtgactgtacctttgtgaacgtaaatgagggtcattctagtggagaaatagccaacacttttgctgctcctgagacgaatcactcttattttgccgctccggagatgaatcctccaagtttcaacacgaaccctaatcagaatcaggaacaagatgctaatcgagatattagcgattggtcgtttttggatccacaggatttggttgatgacgacttcatgaactctctgttcaacaatgacatgaattga

SEQ ID NO: 27 - protein, 626 aa, Camelina sativa
MTFAESLHNKSSVLSINILVVDDDPVILEVLPKMLVKSKYRDPSVMEIKVIAEEDPVKALAILKLQRNNIDLIITDYYMPSMNGLQLKRRITEEIGNLPVIVVSADANKEQESLTCGALGFLPKPIQATDLPKIYQLALTCKRNGKSNYKADDTGASVPQQIKMLPEQANVLKTRKKYSSKAASRSLNSTNGSGLISTDGSRKNRKRKPNGGSEDGAPSQPSKKTKISWTDGLNDLFLQAIRHIGLDKAVPKKILAFMNVPYLTRENVASHLQKYRIFLRKVADQGLMSIMSARGIDSMFRHTQIKESYFNRYTPSTSLYDTSLNNRSIYSKPGQGFGQSRLLSTTREPPVRFNQMPYNYMNRSSTYETHHVGSGSNLTLPIQSNLSLANQPFQNEELRSFFKPPVMANKIGQTSQVHGFGQLGPLAINFNNNMMSSFGSFTPNQPGLGHFSYGMQSFLNNENTAYNPEPHANATTQPNLELPQLEDLNIHSGLGKTNELPYNTSNFQSDHNKQQQGEEASNKFELPSFFSAELNQFLSLEDDSDCTFVNVNEGHSSGEIANTFAAPETNHSYFAAPEMNPPSFNTNPNQNQEQDANRDISDWSFLDPQDLVDDDFMNSLFNNDMN

SEQ ID NO: 28 - protein, 825 aa, Arabidopsis thaliana
MDISNEASVDPFSIGPSSIMGRTIAFRVLFCRSMSQLRRDLFRFLLHWFLRFKLTVSPFVSWFHPRNPQGILAVVTIIAFVLKRYTNVKIKAEMAYRRKFWRNMMRTALTYEEWAHAAKMLEKETPKMNESDLYDEELVKNKLQELRHRRQEGSLRDIMFCMRADLVRNLGNMCNSELHKGRLQVPRHIKEYIDEVSTQLRMVCNSDSEELSLEEKLSFMHETRHAFGRTALLLSGGASLGAFHVGVVRTLVEHKLLPRIIAGSSVGSIICAVVASRSWPELQSFFENSLHSLQFFDQLGGVFSIVKRVMTQGALHDIRQLQCMLRNLTSNLTFQEAYDMTGRILGITVCSPRKHEPPRCLNYLTSPHVVIWSAVTASCAFPGLFEAQELMAKDRSGEIVPYHPPFNLDPEVGTKSSSGRRWRDGSLEVDLPMMQLKELFNVNHFIVSQANPHIAPLLRLKDLVRAYGGRFAAKLAHLVEMEVKHRCNQVLELGFPLGGLAKLFAQEWEGDVTVVMPATLAQYSKIIQNPTHVELQKAANQGRRCTWEKLSAIKSNCGIELALDDSVAILNHMRRLKKSAERAATATSSSHHGLASTTRFNASRRIPSWNVLARENSTGSLDDLVTDNNLHASSGRNLSDSETESVELSSWTRTGGPLMRTASANKFIDFVQSLDIDIALVRGFSSSPNSPAVPPGGSFTPSPRSIAAHSDIESNSNSNNLGTSTSSITVTEGDLLQPERTSNGFVLNVVKRENLGMPSIGNQNTELPESVQLDIPEKEMDCSSVSEHEEDDNDNEEEHNGSSLVTVSSEDSGLQEPVSGSVIDA

SEQ ID NO: 29 - protein, 801 aa, Arabidopsis thaliana
MDISNEAGVDAFSIIGPTTIIGRTIAVRILFCNSVSIFRHKVFRILKFFLRGGRVLLSPFVSLLHPRNPQGILVMVTTMAFLLNRYTSLKAKAEMAYRRKFWRNMMRAALTYEEWSHAAKMLDKETPKVNETDLFDVELVSNKLDELKHRRHEGSLRDIIFCMRADLVRNLGNMCNPELHKGRLHVPRLIKEYIDEVSTQLRMVCDMDTEELSLEEKLSFMHETRHAYGRTALLLSGGASLGAFHLGVVKTLVEHKLLPRIIAGSSVGSVMCAVVGTRSWPELQSFFEGSWHALQFFDQMGGIFTTVKRVMTQGAVHEIRHLQWKLRNLTNNLTFQEAYDITGRILGITVCSLRKHEPPRCLNYLTSPHVVIWSAVTASCAFPGLFEAQELMAKDRTGEIVPYHPPFNLDPEEGSASVRRWRDGSLEMDLPMIQLKELFNVNHFIVSQANPHIAPFLRMKEFVRACGGRFAAKLAQLAEMEVKHRCNQVLELGLPLREVASLFAQEWEGDVTIVMPATFSQYLKIIQNPSNVEIQKAANQGRRCTWEKLAVIKANFGIELALDECVTVLNHMRRLKRSAERAAAFSAISSSPPSKHLLAGTNRFNASKRIPSWNCIARQNSSGSVDDDVLAEASRLYQHIVVGSGRNSNRTSNLSHTYDAGSECDSPEAEDWTRSGGPLMRTNSAQMFTDYVQNLDAVDPEQIRASENDSIVAASSSSHSITVTEGDYLQTGRTHNGFVLNLVRGENLRMNSEPEDSQNESEIPETPESVQLDSPEKDIIDGESSASEDGDAQANLIHDHE

SEQ ID NO: 30 - protein, 826 aa, Camelina sativa
  misc_feature (763)..(763): Xaa can be any naturally occurring amino acid
MDISNEASVDPFSIGPTSIMGRTIAFRVLFCRSMAQLRRDLFRFLLHWFLRFKLTVSPFVSWFHPRNPQGILAVVTIIAFVLKRYTNVKIKAEMAYRRKFWRNMMRTALTYEEWAHAAKMLEKETPKLNESDLYDEELVKNKLQELRHRRQEGSLRDIMFCMRADLVRNLGNMCNSELHKGRLQVPRHIKEYIDEVSTQLRMVCNSDSEELSLEEKLSFMHETRHAFGRTALLLSGGASLGAFHVGVVRTLVEHKLLPRIIAGSSVGSIICAVVASRSWPELQSFFENSLHSLQFFDQLGGVFSIVKRVMTQGALHDIRQLQCMLRNLTSNLTFQEAYDMTGRILGITVCSPRKHEPPRCLNYLTSPHVVIWSAVTASCAFPGLFEAQELMAKDRSGEIVPYHPPFNLGPEVGTKSSGRRWRDGSLEVDLPMMQLKELFNVNHFIVSQANPHIAPLLRLKDLVRAYGGRFAAKLAHLVEMEVKHRCNQVLELGFPLGGLAKLFAQEWEGDVTVVMPATLAQYSKIIQNPTHLDLQKAANQGRRCTWEKLSAIKSNCGIELALDDSVAILNHMRRLKKSAERAASATSSSHHGLASTTRFNASRRIPSWNVIARENSTGSLDDLAADNNNLHASSGRNLSDSETESVELSSSWTRTGGPLMRTASANKFIDFVQSLDIDIALARGFSSSPNSPAVPPGGPFSPSARSMSAHSDSEPNSNSNNNTTSRITVTEGDLLQPERTSNGFLGGVFSIVKRVMTQGALHXELPESVQLDIPEKEMDNSSVSEHEVDDDDEQEDNNGATVSSPVTESSEDSGLQEPVSGSVIDG

SEQ ID NO: 31 - protein, 827 aa, Camelina sativa
MDISNEASVDPFSIGPTSIMGRTIAFRVLFCRSMAQLRRDLFRFLLHWFLRFKLTVSPFLSWFHPRNPQGILAVVTIIAFLLKRYTNVKIKAEMAYRRKFWRNMMRTALTYEEWAHAAKMLEKETPKLNESDLYDEELVKNKLQELRHRRQEGSLRDIMFCMRADLVRNLGNMCNSELHKGRLQVPRHIKEYIDEVSTQLRMVCNSDSEELSLEEKLSFMHETRHAFGRTALLLSGGASLGAFHVGVVRTLVEHKLLPRIIAGSSVGSIICAVVASRSWPELQSFFENSLHSLQFFDQLGGVFSIVKRVMTQGALHDIRQLQCMLRNLTSNLTFQEAYDMTGRILGITVCSPRKHEPPRCLNYLTSPHVVIWSAVTASCAFPGLFEAQELMAKDRSGEIVPYHPPFNLDPEVGTKSSGRRWRDGSLEVDLPMMQLKELFNVNHFIVSQANPHIAPLLRLKDLVRAYGGRFAAKLAHLVEMEVKHRCNQVLELGFPLGGLAKLFAQEWEGDVTVVMPATLAQYSKIIQNPTHVDLQKAANQGRRCTWEKLSAIKSNCGIELALDDSVAILNHMRRLKKSAERAASATSSSHHGLASTTRFNASRRIPSWNVIARENSTGSLDDLVADNNNLHASSGRNLSDSETESVELSSWTRTGGPLMRTASANKFIDFVQSLDIDIALARGFSSSPNSPAVPPGGPFSPSARSMSAHSDNEPNSNSNNNTTSRITVTEGDLLQPERTSNGFVLNVVKRENLGMSSIGGNQNTELPESVQLDIPEKEMDNSSVSEHEVDDDDKQEDNNGATVSSPVTESSEDSGLQEPVSGSVIDG

SEQ ID NO: 32 - protein, 830 aa, Camelina sativa
MDISNEASVDPFSIGPTSIMGRTIAFRVLFCRSMAQLRRDLFRFLLHWFLRFKLTVSPFVSWFHPRNPQGILAVVTIIAFVLKRYTNVKIKAEMAYRRKFWRNMMRTALTYEEWAHAAKMLEKETPKLNESDLYDEELVKNKLQELRHRRQEGSLRDIMFCMRADLVRNLGNMCNSELHKGRLQVPRHIKEYIDEVSTQLRMVCNSDSEELSLEEKLSFMHETRHAFGRTALLLSGGASLGAFHVGVVRTLVEHKLLPRIIAGSSVGSIICAVVASRSWPELQSFFENSLHSLQFFDQLGGVFSIVKRVMTQGALHDIRQLQCMLRNLTSNLTFQEAYDMTGRILGITVCSPRKHEPPRCLNYLTSPHVVIWSAVTASCAFPGLFEAQELMAKDRSGEIVPYHPPFNLGPEVGTKSSGRRWRDGSLEVDLPMMQLKELFNVNHFIVSQANPHIAPLLRLKDLVRAYGGRFAAKLAHLVEMEVKHRCNQVLELGFPLGGLAKLFAQEWEGDVTVVMPATLAQYSKIIQNPTHLDLQKAANQGRRCTWEKLSAIKSNCGIELALDDSVAILNHMRRLKKSAERAASATSSSHHGLASTTRFNASRRIPSWNVIARENSTGSLDDLAADNNNLHASSGRNLSDSETESVELSSSWTRTGGPLMRTASANKFIDFVQSLDIDIALARGFSSSPNSPAVPPGGPFSPSARSMSAHSDSEPNSNSNNNTTSRITVTEGDLLQPERTSNGFVLNVVKRENLGMSSIGGNQNTELPESVQLEIPEKEMDNSSVSEHEVDDDDEHEQEDNNGATVSSPVTESSEDSGLQEPVSGSVIDG

SEQ ID NO: 33 - protein, 810 aa, Camelina sativa
MDISNEAGVDAFSIIGPTSIIGRTIAYRILFCNSVSIFRHKLYRFLKFFFRGGRVLLSPFVSLLHPRNPQGILAMVMTMAFLLNRYTSLKAKAEMAYRRKFWRNMMRAALTYEEWSHAAKMLDKETPKVNEADLFDVELVRNKLEELKHRRHEGSLKDIIFCMRADLVRNLGNMCNPELHKGRLHVPRLIKEYIDEVSTQLRMVCDTDTEELPLEEKLSFMHETRHAYGRTALLLSGGASLGAFHLGVVKTLVEHKLLPRIIAGSSVGSVMCAVVGTRSWPELQSFFEGSWHALQFFDQMGGIFTTVKRVMTQGAVHEIRHLQWKLRNLTNNLTFQEAYDITGRILGITVCSLRKHEPPRCLNYLTSPHVVIWSAVTASCAFPGLFEAQELMAKDRTGEIVPYHPPFNLDPEEGSGESVRRWRDGSLEMDLPMMQLKELFNVNHFIVSQANPHIAPFLRMKEFVRACGGRFAAKLAQLAEMEVKHRCHQVLELGLPLREVASLFAQEWEGDVTIVMPATFSQYLKIIQNPSNVEIQKAANQGRRCTWEKLAVIKANFGIELALDECVAVLNHMRRLKRSAERAAAFSAISSSPPSKHLLAGANRFNASKRIPSWNCIARQNSSGSVDDDVLAEASRLYQHIVVGSGKTSNRTSNLSHSYDAGSEGESPEAEDWTRSGGPLMRTSSAQMFTDYVQNLNAVDPEQTTIGDSDRAPETELNAAAAAASSSSSSSITVTEGDYLQTGRTHNGIVLNLVRGENLRMNQDLEDSETECETPESVQLESPEKDIIDGNSSASEDGDTSTPQANLTHE

SEQ ID NO: 34 - protein, 845 aa, Camelina sativa
MDISNEAGVDAFSIIGPTSIIGRTIAYRILFCNSVSIFRHKLYRFLKFFLRGGRVLLSPFVSLLHPRNPQGILAMVMTMAFLLNRYTSLKAKAEMAYRRKFWRNMMRAALTYEEWSHAAKMLDKETPKVNEADLFDVELVRNKLEELKHRRHEGSLKDIIFCMRADLVRNLGNMCNPELHKGRLHVPRLIKEYIDEVSTQLRMVCDTDTEELPLEEKLSFMHETRHAYGRTALLLSGGASLGAFHLGVVKTLVEHKLLPRIIAGSSVGSVMCAVVGTRSWPELQSFFEGSWHALQFFDQMGGIFTTVKRVMTQGAVHEIRHLQWKLRNLTNNLTFQEAYDITGRILGITVCSLRKHEPPRCLNYLTSPHVVIWSAVTASCAFPGLFEAQELMAKDRTGEIVPYHPPFNLDPEEGSGESVRRWRDGSLEMDLPMMQLKELFNVNHFIVSQANPHIAPFLRMKEFVRACGGRFAAKLAQLAEMEVKHRCHQVLELGLPLREVASLFAQEWEGDVTIVMPATFSQVTTHCKQCIPTRTFHSNSILVLSLSICFALCVSGFQYLKIIQNPSNVEIQKAANQGRRCTWEKLAVIKANFGIELALDECVAVLNHMRRLKRSAERAAAFSAISSSPPPKHLLAGANRFNASKRIPSWNCIARQNSSGSVDDDVLAEASRLYQNIVVGSGKTSNRTSNLSHSYDAGSEGESPEAEDWTRSGGPLMRTSSAQMFTAYVQNLNAADPEQTTIGDSDRAPETELNAAAAASSSSSSSITVTEGDYLQTGRTHNGIVLNLVRGENLRMNQDLEDSENECETPESVQLESPEKDIIDGNSSASEDGDTSTAQANLTHE

SEQ ID NO: 35 - DNA, 2430 bp, Camelina sativa
atggatataagcaatgaagcaggcgttgatgcgttctcaattataggacccacgtctataatcggtagaacaattgcctaccggatcttgttctgcaactcagtgtctatattcagacacaaactctacagatttctcaagtttttcctcagaggaggcagggttttactatcaccgtttgtgtccttactacatcccaggaatccacaagggatattagcaatggtgatgacgatggcctttctgttgaatcgctacacaagcctaaaagcaaaagctgagatggcttacaggaggaaattctggaggaacatgatgagagctgcattgacatacgaggaatggtctcacgccgcaaagatgcttgataaagagactccaaaggtgaacgaggcagatctcttcgatgtggagctcgtaaggaataagcttgaggagcttaagcatagacgtcacgagggatctctaaaagacattatcttctgtatgagagctgatcttgttagaaacctcggtaacatgtgtaaccctgagcttcacaagggaaggcttcatgtgccgagattaattaaagagtacatcgatgaggtttctacacagctcaggatggtttgtgacacggacactgaagagcttcctttggaggagaaactctcttttatgcatgagactagacacgcgtatggaagaacggctctgcttctgagcggaggagcttctctaggagctttccatcttggtgtggtcaagacgcttgtagaacataagctcttgccgagaattatagccggttcaagcgtgggatccgtaatgtgtgcggttgtggggacaaggtcatggcctgagttacagagcttctttgaaggctcttggcatgctttgcagttctttgatcagatgggagggattttcactactgtgaaacgggttatgactcaaggcgcagtccatgagatccggcatttgcaatggaagttgaggaatctcaccaacaaccttacattccaagaagcttacgacataacaggacggattctagggataacagtttgttccttgaggaaacacgaaccccctagatgtctcaattatctgacgtcgcctcatgttgtgatatggagtgcagtgactgcatcttgtgctttcccaggtctttttgaggctcaagaactcatggccaaagatagaactggagagattgttccttatcacccaccttttaacttagatcctgaagagggctcaggagaatcagtacggcgctggagggacggtagtttggagatggacttaccgatgatgcaactgaaagagcttttcaatgtcaaccatttcattgtcagtcaagccaaccctcacatagcacccttcttgaggatgaaggagtttgttagagcttgtggaggtcgatttgctgcaaagctcgctcaactcgcggagatggaagtgaagcatagatgtcatcaagtactagaactcgggcttcctctaagagaagtagcttccctatttgctcaagaatgggaaggtgatgtcactatcgtcatgccagctactttttctcagtacttaaagatcatacagaacccaagcaatgtagagattcaaaaggcggcaaatcaagggaggagatgcacctgggagaaactcgcagtcatcaaagcaaacttcgggatcgaactagcactcgacgagtgcgtcgccgttctcaaccacatgcgccgccttaaacgcagcgctgaaagagccgctgctttctcagccatctcttcctccccaccacccaaacatctcttggccggagccaacagattcaacgcctctaaacgaatcccttcctggaactgcatagctcgtcaaaactcatccggatccgtcgacgatgatgtgctcgcagaagcttcacggttgtaccagaacatcgtggttggatccgggaagactagtaaccgaaccagcaacttaagccattcgtatgacgcaggaagcgaaggagaatcaccagaagctgaagactggacgagatctggtggaccgttgatgaggacatcgtcagctcagatgttcactgcctacgtccaaaatctcaacgccgctgatcctgaacagacgacgattggtgactccgatagggctccggagaccgaattgaacgccgctgctgctgcttcttcttcttcttcttccagcataacggttacagaaggcgattatcttcagacggggaggacacataacggaatcgtgttgaatctcgtcagaggagagaatctgaggatgaatcaggatctggaagatagcgaaaacgaatgtgaaactccggagagcgtgcaactcgaatctccggagaaggacattattgacggaaatagctcggcgtctgaagacggagacactagtactgctcaggcgaatctaacccacgagtga

SEQ ID NO: 36 - protein, 809 aa, Camelina sativa
MDISNEAGVDAFSIIGPTSIIGRTIAYRILFCNSVSIFRHKLYRFLKFFLRGGRVLLSPFVSLLHPRNPQGILAMVMTMAFLLNRYTSLKAKAEMAYRRKFWRNMMRAALTYEEWSHAAKMLDKETPKVNEADLFDVELVRNKLEELKHRRHEGSLKDIIFCMRADLVRNLGNMCNPELHKGRLHVPRLIKEYIDEVSTQLRMVCDTDTEELPLEEKLSFMHETRHAYGRTALLLSGGASLGAFHLGVVKTLVEHKLLPRIIAGSSVGSVMCAVVGTRSWPELQSFFEGSWHALQFFDQMGGIFTTVKRVMTQGAVHEIRHLQWKLRNLTNNLTFQEAYDITGRILGITVCSLRKHEPPRCLNYLTSPHVVIWSAVTASCAFPGLFEAQELMAKDRTGEIVPYHPPFNLDPEEGSGESVRRWRDGSLEMDLPMMQLKELFNVNHFIVSQANPHIAPFLRMKEFVRACGGRFAAKLAQLAEMEVKHRCHQVLELGLPLREVASLFAQEWEGDVTIVMPATFSQYLKIIQNPSNVEIQKAANQGRRCTWEKLAVIKANFGIELALDECVAVLNHMRRLKRSAERAAAFSAISSSPPPKHLLAGANRFNASKRIPSWNCIARQNSSGSVDDDVLAEASRLYQNIVVGSGKTSNRTSNLSHSYDAGSEGESPEAEDWTRSGGPLMRTSSAQMFTAYVQNLNAADPEQTTIGDSDRAPETELNAAAAASSSSSSSITVTEGDYLQTGRTHNGIVLNLVRGENLRMNQDLEDSENECETPESVQLESPEKDIIDGNSSASEDGDTSTAQANLTHE

SEQ ID NO: 37 - protein, 806 aa, Camelina sativa
MDISNEAGVDAFSIIGPTSIIGRTIAYRILFCNSVSIFRHKLYRFLKFFLRGGRVLLSPFVSLLHPRNPQGILAMVMTMAFLLNRYTSLKAKAEMAYRRKFWRNMMRAALTYEEWSHAAKMLDKETPKVNESDLFDVELVRNKLEELKHRRHEGSLKDIIFCMRADLVRNLGNMCNPELHKERLHVPRLIKEYIDEVSTQLRMVCDTDTEELPLEEKLSFMHETRHAYGRTALLLSGGASLGAFHLGVVKTLVEHKLLPRIIAGSSVGSVMCAVVGTRSWPELQSFFEGSWHALQFFDQMGGIFTTVKRVMTQGAVHEIRHLQWKLRNLTNNLTFQEAYDITGRILGITVCSLRKHEPPRCLNYLTSPHVVIWSAVTASCAFPGLFEAQELMAKDRTGEIVPYHPPFNLDPEEGSGESLRRWRDGSLEMDLPMMQLKELFNVNHFIVSQANPHIAPFLRMKEFVRACGGRFAAKLAQLAEMEVKHRCHQVLELGLPLREVASLFAQEWEGDVTIVMPATFSQYLKIIQNPSNVEIQKAANQGRRCTWEKLAVIKANFGIELALDECVAVLNHMRRLKRSAERAAAFSAISSSPPSKHLLAGANRFNASKRIPSWNCIARQNSSGSVDDDVLAEASRLYQHIVVGSGKPSNRTSNLSHAGSEVESPEAEDWTRSGGPLMRTSSAQMFTDYVQNLNAAADPEQTTIGGDSDRAPEVELNAAAASPSSSITVTEGDYLQTGRTHNGFVLNLVRGENLRMNQDLEDSENECETPESVQLESPEKDIIDGNSSASEDGDTSTTAQANLTHE

SEQ ID NO: 38 - DNA, 2352 bp, Brassica napus
  misc_feature (703)..(703): n is a, c, g, or t
  misc_feature (2266)..(2267): n is a, c, g, or t
atggatcatataagcaacgaggctaatgtagatcccttctcaatcggaccaacatccatcattggtcgaaccatcgccttcagagtcttgttctgcaaatcaataacacagctcagacgcgatctcctacgcttcttcctccattggttccgtacatttaagctggttattacacccttcgtgtcgtggttccacccccggaaaaacccgcaagggatcttagccgtcgtcacagtcatcgccttcgcgctgaaacgttacaccaacgtgaagataaagtcagagatggcttaccgaagaaagttctggaggaacatgatgcgaactgctctgacttacgaggaatggtctcacgccgctaagatgctggagaaggagacaacaacgacttcgaagatgttaaacgagtctgatctttacgacgaagagttggttaagaacaaactcaacgagcttctccaccgtcgccaagaagcgtcccttagagagatcatgttctgcatgagggccgatctcgtgaggaacctcggtaacatgtgtaactctgagcttcacaaaggaagactccaggttcctaggcttatcaaagagtatatagacgaggtctctactcagctgagaatggtctgtaacaactctgactcgttagaggatctttctttagatgagaagctttcttttatgcacgagacgcgccacgccnccgctctgcttctaagcggcggggcgtctctcggcgcgtttcatgtaggagtggtcaagactcttgtcgagcacaagctcttgcctcggatcattgctggctcaagcgtcggctcgataatctgctctgtcgtggcgtcaagatcctggcccgagctacaaggcttcttcgagaactctctgcagtccttacagttctttgaccagctcggaggcgttttcacgatcgtgaaacgcgtcatgacgcaaggggcgttacacgatataagacagttgcagtgcatgcttaggagcctcacgtgcaacctcacgttccaagaggcttatgatctaacggggaggatactggggataacggtttgctccccgaggaagcacgagccgcctcggtgtcttaactatttgacttcgcctcacgtggttatatggagcgcggtgactgcttcttgcgcctttcctggtctctttgaggctcaggagctaatggctaaagatagaagtggagagattgtgccgtatcatccgccttttaatttggatcctgaagaaggtactgaaccgtctgcacgcaggtggagagatggtagcttggaggttgatttgccgatgatgcagcttaaggagctgtttaatgttaatcattttattgtgagtcaagctaatcctcacattgctccattactgcgtataaaggatttagttcgagcttatggtggaagattcgctgccaagcttgcgcatctagtggagatggaggttaaacatagatgcaaccaggttttagagcttggctttccactcggtggactcgcaaagctctttgctcaagagtgggaaggtgatgtaacggttgtaatgcctgctactcttgctcaatactcaaagatcatacaaaaccctactcacgtggagcttcaaaaagcagctaaccaaggaaggagatgcacttgggagaagctttcagccattaaagcaaactgcgggatcgagcttgcgcttgatgagtgtgtagctgtccttaaccatatgcgtaggctcaagagaagcgccgagagagcagcctcgtctcatcacggtttggcttcaaccacaaggttcaatgcttctaaaagaatcccttcttggaacgttattgcaagagagaactctactggttctcttgatgaactagtcgccgacagtaatctcaggaacttgagtgatagtgaaacggagagtgtggagctgagttcttggacaagaactggtgggcctttgatgagaacagcttctgctaataagttcattgatttcgttcagagtcttgatgtggacattgcattggccagagggtttagtagcagccccagttctccagcggcaaacacttcaagcataacagttactgaaggtgattttctacagcctgagagaacgagtaacggtattgtgttgaacgtggttagaagagaagacttgggaatgtctgtcgggaaccagaacactgagctgccggagagtgtgcagcttgacatacctgagaaggagatggataatagctctgtatccgaacatgaagtggatnngaatgatgaagaataccaacataacgggtcggttactgtatcttcagacgattcaggtttacaagaaccggtgtctggtatttga

SEQ ID NO: 39 - protein, 783 aa, Brassica napus
  misc_feature (235)..(235): Xaa can be any naturally occurring amino acid
  misc_feature (756)..(756): Xaa can be any naturally occurring amino acid
MDHISNEANVDPFSIGPTSIIGRTIAFRVLFCKSITQLRRDLLRFFLHWFRTFKLVITPFVSWFHPRKNPQGILAVVTVIAFALKRYTNVKIKSEMAYRRKFWRNMMRTALTYEEWSHAAKMLEKETTTTSKMLNESDLYDEELVKNKLNELLHRRQEASLREIMFCMRADLVRNLGNMCNSELHKGRLQVPRLIKEYIDEVSTQLRMVCNNSDSLEDLSLDEKLSFMHETRHAXALLLSGGASLGAFHVGVVKTLVEHKLLPRIIAGSSVGSIICSVVASRSWPELQGFFENSLQSLQFFDQLGGVFTIVKRVMTQGALHDIRQLQCMLRSLTCNLTFQEAYDLTGRILGITVCSPRKHEPPRCLNYLTSPHVVIWSAVTASCAFPGLFEAQELMAKDRSGEIVPYHPPFNLDPEEGTEPSARRWRDGSLEVDLPMMQLKELFNVNHFIVSQANPHIAPLLRIKDLVRAYGGRFAAKLAHLVEMEVKHRCNQVLELGFPLGGLAKLFAQEWEGDVTVVMPATLAQYSKIIQNPTHVELQKAANQGRRCTWEKLSAIKANCGIELALDECVAVLNHMRRLKRSAERAASSHHGLASTTRFNASKRIPSWNVIARENSTGSLDELVADSNLRNLSDSETESVELSSWTRTGGPLMRTASANKFIDFVQSLDVDIALARGFSSSPSSPAANTSSITVTEGDFLQPERTSNGIVLNVVRREDLGMSVGNQNTELPESVQLDIPEKEMDNSSVSEHEVDXNDEEYQHNGSVTVSSDDSGLQEPVSGI

SEQ ID NO: 40 - DNA, 2469 bp, Brassica napus
atggatataagcaacgaggccaatgtcgatcccttctcaatcggaccaacctccatcctcggccgaaccatcgccttccgagtcctcttctgcaaatcaatgctccagctccgccgcgacctcttccgcttcctcctccactggttcctcacactcaagctcgccgtctccccctttgtctcctggttccacccccggaacccccaggggatcctcgccgtcgtcacgatcatcgccttcgtcctgaaacgctacaccaacgtgaaggccaaggccgagatggcctaccgtagaaagttctggaggaacatgatgcgcgcggcgttgacttacgaggaatgggctcacgccgctaagatgttggataaagagactccgaagatgaacgaatccgatctttacgatgaagagttggttaagaacaagctaatggagcttcgtcatcgacgtcaagaaggctctctgagagacattatgttctgtatgagagctgatctcgtgaggaacctcgggaacatgtgtaaccctgagctgcacaaagggagactccaagttcctagacatatcaaagagtatatagacgaagtctctactcagctgagaatggtatgcaacaactctgagtccttagaagacctttctttagatgagaagctttctttcatgcacgagacgcgtcacgcctttggtaggactgctttgctactaagtggcggggcctctctcggcgcgtttcatgtcggtgttgtcaggactcttgtcgagcataagcttttacctcggatcatcgccggctctagcgtcggctcaataatctgctccgtcgtggcgtcaagatcctggccagaactgcagagcttcttcgagaactcgttacactctctccagttctttgaccagctaggaagcgtcttcacgatcgtgaaacgcgtcatgacgcaaggagcgttgcacgacattagacagctgcagtgcatgcttaggaacctcacgtgcaacctcacgttccaagaggcttatgatctgacggggaggatactcgggataacggtttgctcaccgaggaagcacgagccgcctcggtgtcttaactatctcacttctcctcacgtggtgatatggagcgcggtgactgcttcttgcgctttccctggtttgtttgaagctcaggagctgatggctaaagataggagtggggagatagtgccgtatcatccgccttttaatttggaaccggaggaaggtggggataagtcgtctacgaggaggtggagagatgggagtttggaggttgatttgccgatgatgcagcttaaggagctgtttaatgttaatcattttattgtgagccaggctaatcctcacattgctccgttgctgcgtttgaaggatatagttagagcttatggtggtagattcgctgctaagcttgcgcatctagtggagatggaggtgaaacatagatgcaaccaagtactagagcttggctttccactcggtggactcgctaagctatttgctcaggagtgggaaggtgatgtaacagttgtgatgcctgctactcttgctcagtactctaagatcatacagaaccctacacacgtggagctacaaaaggcagctaaccaaggaaggagatgcacttgggagaagctctcagccatcaaagcaaactgcgggatcgagcttgcgcttgacgagtgcgtagccattctcaaccacatgcggaggctcaagagaagcgcagagagagcctccacagcttcctcgtctcatcacggattagcttcaacgacaagattcaatgcttctagaagaatcccgtcttggaacgtcatcgctagagagaactcaactggctcactggacgacctcgttactgacagtaacaataataatctccacgcggggaggaacctaagcgacagcgaaacggagagcgtggagatgagttcttggactaggactggtggaccgttgatgagaacggcttctgctaataggttcactgactttgtccatggtcttgacgtggacattgcgttgacaagagggtttactagcagccctaactctccagcggttcctggcccggttagtccgagttttagtccgagatcgagatccttggcggctcaatccgagagcgaatctgacaagagggaaagtagcaacagttcgagtataacagttagtgaaggtgatcttctacagcctgagagaacgagtaacggttttgttttaaacgttgttagaagagagaacttggggatgcctgtggggaaccagagcggtgagctgccggagagtgtacagatagatatacctgagagggagatggataatagctctgtctcagaacatgaagatgataatgatgatgatgatgatgaagaagaagaacataagggctcggttccggttaaagattccggtttacaagattcttgtagtgtaatagatgcttag

SEQ ID NO: 41 - protein, 822 aa, Brassica napus
MDISNEANVDPFSIGPTSILGRTIAFRVLFCKSMLQLRRDLFRFLLHWFLTLKLAVSPFVSWFHPRNPQGILAVVTIIAFVLKRYTNVKAKAEMAYRRKFWRNMMRAALTYEEWAHAAKMLDKETPKMNESDLYDEELVKNKLMELRHRRQEGSLRDIMFCMRADLVRNLGNMCNPELHKGRLQVPRHIKEYIDEVSTQLRMVCNNSESLEDLSLDEKLSFMHETRHAFGRTALLLSGGASLGAFHVGVVRTLVEHKLLPRIIAGSSVGSIICSVVASRSWPELQSFFENSLHSLQFFDQLGSVFTIVKRVMTQGALHDIRQLQCMLRNLTCNLTFQEAYDLTGRILGITVCSPRKHEPPRCLNYLTSPHVVIWSAVTASCAFPGLFEAQELMAKDRSGEIVPYHPPFNLEPEEGGDKSSTRRWRDGSLEVDLPMMQLKELFNVNHFIVSQANPHIAPLLRLKDIVRAYGGRFAAKLAHLVEMEVKHRCNQVLELGFPLGGLAKLFAQEWEGDVTVVMPATLAQYSKIIQNPTHVELQKAANQGRRCTWEKLSAIKANCGIELALDECVAILNHMRRLKRSAERASTASSSHHGLASTTRFNASRRIPSWNVIARENSTGSLDDLVTDSNNNNLHAGRNLSDSETESVEMSSWTRTGGPLMRTASANRFTDFVHGLDVDIALTRGFTSSPNSPAVPGPVSPSFSPRSRSLAAQSESESDKRESSNSSSITVSEGDLLQPERTSNGFVLNVVRRENLGMPVGNQSGELPESVQIDIPEREMDNSSVSEHEDDNDDDDDEEEEHKGSVPVKDSGLQDSCSVIDA

SEQ ID NO: 42 - DNA, 2421 bp, Brassica napus
atggatcacataagtaacgaggccaatgtcgatcccttttcaatcggaccaacatccctcatcggccgaaccatagccttcagagtcctcttctgcaaatcaatgctccagctcagacgcgacctcttccgcttcttgctgcactggttccttacactcaagccttccgtctcaccctttgtatcctggttccacccccggaaccctcaagggatcttagcagtggtcacaatcatcgccttcgtgttgaaacgttacaccaacgtgaagaagaaggcggagatggtttactggaggaacttctggaggaacacgatgcgcgcggctttgacttacgaggaatgggctcacgccgctaagatgctggagaaggagacgccgaagatgaacgaatctgatctctacgacgaagagttggttaaaaacaagcttcgggagctttgtcatcgtcgccgagaaggttgtcttagagacatcatgttctgtatgagagctgatcttgtcaggaacctcggtaacatgtgtaactcggagctgcatcaagggaggcttcaggttcctaggcttatcaaagagtatatcaacgaggtctctactcagctgagaatggtttgctcctctgagtcattagaagagctttctttagaagagaagctttctttcatgcacgagacacgtcacgcctttggtagaacggctttgcttctcagcggtggggcttctctaggcgcgtttcatgtcggtgtggttaggactctggtggagcataagctcttacctcggattattgctggctctagcgttggttccataatctgttcaatcgtagccacgaggtcttggcccgagctacagagtttctttgagaactcattgcactcactacagttctttgatcagctaggaagcgttttcacgatcgttaaaagagtcatgacgcaaggcgcgttacacgatataagacagttgcaatgcatgcttaggaacctcacgttcaatctcacgttccaagaggcttatgatataacagggaggatcctagcgataacggtttgctccccaaggaagcatgagccgcctcggtgtcttaactacttgacttcgcctcatgtggttatatggagtgcggttactgcttcttgtgcttttcctggtctctttgaagctcaggagcttatggctaaagatagaagtggagagattgtgccgtatcatccaccttttaatttggatcctgaagaaggtactgaaccatatgcacgcaggtggagagatgggagtttggaggttgatttgccgatgatgcagcttaaggagctcttcaatgttaatcattttattgtgagccaagctaatcctcacattgttccgttactgcgtattaaggatatagttcgagcttatggtggtagattcgcagcaaagcttgcgcatctagtggagatggaggttaaacatagatgcaaccaagtactagaacttggctttccactcggtggactcgcaaagctttttgctcaggagtgggaaggtgatgtaacagttgtaatgccagctactcttgctcagtactcaaagattatacaaaaccctactcatgtggagcttcaaaaagcggctaaccaaggaaggagatgcacttgggagaagctatcagccataaaagcaaactgtgggatcgagcttgcgcttgatgagtgtgtaactaatcttaaccatatgcgtaggctcaacagaagcgctgagagagccgctgctgctgctggcacgtcctcctcgtctcatcacggattagcttcaaccacaagattcaatggttctagaagaatcccatcttggaacgtaattgccagagagaactcaacgggttcactcgatgaactagtcgctgagagtaatctccacgcgtcttcgggctggaacttgagcgatagtgaaactgagagcgtggagttgagttcttggacaaggactggtggacctttgatgagaacagcttctgctaataagttcattgacttcgttcagagtcttgatgtagacattgcactaagcagaggatttagtagcagccccacttctccggcggttcctggcccatttagcccaagctttagtccagattcaagatccatggacaacaataacaacatgcttggagcaaacacttcaagcataatagttactgaaggtgatcttctacagactgagaggacgagtaacggttttgtgttgaacgttgttagaagagaggacttgggaatgtctattgagaataagaacactgagctgccagagagtgtacagctcgatatacctgacaaggagatggatgatagctctgtatcagaacatgaagtagaccaagaagatgatgatgatgaagaacatggcggctcggatccggttaacgtttcttga

SEQ ID NO: 43 - protein, 806 aa, Brassica napus
MDHISNEANVDPFSIGPTSLIGRTIAFRVLFCKSMLQLRRDLFRFLLHWFLTLKPSVSPFVSWFHPRNPQGILAVVTIIAFVLKRYTNVKKKAEMVYWRNFWRNTMRAALTYEEWAHAAKMLEKETPKMNESDLYDEELVKNKLRELCHRRREGCLRDIMFCMRADLVRNLGNMCNSELHQGRLQVPRLIKEYINEVSTQLRMVCSSESLEELSLEEKLSFMHETRHAFGRTALLLSGGASLGAFHVGVVRTLVEHKLLPRIIAGSSVGSIICSIVATRSWPELQSFFENSLHSLQFFDQLGSVFTIVKRVMTQGALHDIRQLQCMLRNLTFNLTFQEAYDITGRILAITVCSPRKHEPPRCLNYLTSPHVVIWSAVTASCAFPGLFEAQELMAKDRSGEIVPYHPPFNLDPEEGTEPYARRWRDGSLEVDLPMMQLKELFNVNHFIVSQANPHIVPLLRIKDIVRAYGGRFAAKLAHLVEMEVKHRCNQVLELGFPLGGLAKLFAQEWEGDVTVVMPATLAQYSKIIQNPTHVELQKAANQGRRCTWEKLSAIKANCGIELALDECVTNLNHMRRLNRSAERAAAAAGTSSSSHHGLASTTRFNGSRRIPSWNVIARENSTGSLDELVAESNLHASSGWNLSDSETESVELSSWTRTGGPLMRTASANKFIDFVQSLDVDIALSRGFSSSPTSPAVPGPFSPSFSPDSRSMDNNNNMLGANTSSIIVTEGDLLQTERTSNGFVLNVVRREDLGMSIENKNTELPESVQLDIPDKEMDDSSVSEHEVDQEDDDDEEHGGSDPVNVS

SEQ ID NO: 44 - DNA, 2283 bp, Brassica napus
atggatcgtataagcaacgaagccaatgtagatcccttctcaatcggaccaacatccatcattggtcgaaccatcgccttcagagtcttgttctgcaagtcaataacacagctcagacgcgatctctttcgcttcttcctccattggttccgtacattaaagctggttattacacccttcgtgtcgtggttccacccccggcaaaacccgcaagggatcttagccgtcgtcacagtcatcgccttcgcgctaaaacgttacacgaacgtgaagataaagtcagagatggcttaccgaagaaagttctggaggaacatgatgcgcgcggctctgacttacgaggaatggtctcacgccgctaagatgctggagaaggagacaacaacaacttcgaagacgttaaacgagtctgatctttacgacgaagagctggttaagaacaagctcaacgagcttctccaccgtcgccaagaaggttccctcagagacatcatgttctgcatgagagccgatctcgtgaggaacctcggtaacatgtgtaactctgagcttcacaaaggaaggctccaggttcctaggcttatcaaagagtatatagacgaggtctctactcagctgagaatggtttgtaacaactctgactcctcagaggatctttctttagatgagaagctttcttttatgcacgagacgcgccacgcctttggtagaacggctctgcttctaagcggcggtgcctctctcggggcgtttcacgtaggagtggtcaagactcttgtcgagcacaagctcttgcctcggatcatcgctggctctagcgtcggctcgataatctgctcggtcgttgcctcgaggtcctggcccgagctgcaaggcttcttcgagaactctctgcagtccttacagttctttgaccagctcggaggcgttttcacgatcgtgaaacgtgtcatgacgcaaggggcgttacacgatatcagacagctgcagtgcatgcttaggagcctcacgtgcaacctcacgttccaagaggcttatgatctaacggggaggatacttgggataacggtttgctccccgaggaagcacgagccgccacggtgtcttaactatttgacttcgcctcatgttgttatatggagcgcggtgactgcttcttgcgcctttcctggtctctttgaggctcaggagctgatggctaaagatagaagtggagagattgtgccgtatcatccaccttttaatttggatcctgaagaaggtactactgaaccgtctgcacgcaggtggagagatggtagcttggaggttgatttgccgatgatgcagcttaaggagctgtttaatgttaatcattttattgtgagccaagctaatcctcacattgctccgttgctgcgtataaaggatttggttcgagcttatggtggtagattcgctgccaagcttgcgcatctagtggagatggaggttaaacatagatgcaaccagatactagagcttggctttccactccgtggactcgcaaagctctttgctcaagagtgggaaggtgatgtaacagttgtaatgcctgccactcttgctcagtactcaaagattatacaaaaccctactcacgtagagcttcagaaagcggctaaccaaggaaggaggtgcacttgggagaagctttcagccattaaagcaaactgcgggatcgagcttgcgcttgatgagtgtgtagctgtccttaaccatatgcgtaggctcaagagaagtgccgagagagcagcttcgtctcatcacggtttggcttcaaccactaggttcaatgcttctaaaagaatcccttcttggaacgttattgcaagagagaactctactggttctcttgatgaactagtcgccgactgtaatctcaggaacttgagtgatagtgaaacggagagcgtggagttgagttcctggacaagaactggtgggcctttgatgagaacagcttctgctaataagttcattgattttgttcagagtcttgatgtcgacattgcattggccagagggtttagtagcagccccagttctcccgcggcaaacacttcaagcataacggttactgaaggtgattttctacagcctgagagaacgagtaacggtattgtgttgaacgttgttagaagagaagacttgggaatgtctgtcgagaaccagaacactgagctgccggagagtgtgcagcttgacatacctgagaaggagatggataattgctctgaatcagaacatgaagtggatgaatga

SEQ ID NO: 45 - protein, 760 aa, Brassica napus
MDRISNEANVDPFSIGPTSIIGRTIAFRVLFCKSITQLRRDLFRFFLHWFRTLKLVITPFVSWFHPRQNPQGILAVVTVIAFALKRYTNVKIKSEMAYRRKFWRNMMRAALTYEEWSHAAKMLEKETTTTSKTLNESDLYDEELVKNKLNELLHRRQEGSLRDIMFCMRADLVRNLGNMCNSELHKGRLQVPRLIKEYIDEVSTQLRMVCNNSDSSEDLSLDEKLSFMHETRHAFGRTALLLSGGASLGAFHVGVVKTLVEHKLLPRIIAGSSVGSIICSVVASRSWPELQGFFENSLQSLQFFDQLGGVFTIVKRVMTQGALHDIRQLQCMLRSLTCNLTFQEAYDLTGRILGITVCSPRKHEPPRCLNYLTSPHVVIWSAVTASCAFPGLFEAQELMAKDRSGEIVPYHPPFNLDPEEGTTEPSARRWRDGSLEVDLPMMQLKELFNVNHFIVSQANPHIAPLLRIKDLVRAYGGRFAAKLAHLVEMEVKHRCNQILELGFPLRGLAKLFAQEWEGDVTVVMPATLAQYSKIIQNPTHVELQKAANQGRRCTWEKLSAIKANCGIELALDECVAVLNHMRRLKRSAERAASSHHGLASTTRFNASKRIPSWNVIARENSTGSLDELVADCNLRNLSDSETESVELSSWTRTGGPLMRTASANKFIDFVQSLDVDIALARGFSSSPSSPAANTSSITVTEGDFLQPERTSNGIVLNVVRREDLGMSVENQNTELPESVQLDIPEKEMDNCSESEHEVDE

SEQ ID NO: 46 - DNA, 2463 bp, Brassica napus
atggatataagcaacgaagccaatgtcgatcccttctcaatcggaccaacctccatcctcggccgaaccatcgccttcagagtcctcttctgcaaatcaacgctccagctccgccgcgacctcttccgcttcctcctccactggttcctcacactcaagctcgccgtctcccccttcgtctcctggttccacccccggaacccccaggggatcctagccgtcgtcacgatcatcgccttcgtcctaaaacgctacacgaacgtcaaggccaaggcggagatggcgtaccgtaggaagttctggaggaacatgatgcgcgcggcgttgacaaacgaggaatgggctcacgccgctaagatgctggacaaggagactccgaagatgaacgaatccgatctttacgacgaggagttggttaagaacaagctaatggagcttcgtcatcgacgccaagaaggaagccttagagacattatgttctgtatgagagctgatctggtaaggaacctcgggaacatgtgtaactctgagctacacaaaggtagactccaagttccgagacatatcaaagagtatatagacgaagtctctactcagctgagaatggtatgcaacaactctgactcgttagaagacctttctttagatgagaagctttcttttatgcacgagacgcgtcacgcctttggtagaacggcgttgctactcagcggcggggcttctctaggcgcgtttcatgtcggcgttgtcaggactctcgtcgagcataagcttttgcctcggatcatcgccggctctagcgtcggctcaatcatctgctcggtcgtagcgtcaagatcctggcccgagctgcagagcttcttcgagaactcgttacattctctccagttctttgaccagctaggaagcgtcttcacgatcgtgaaacgcgtcatgacgcagggagcgttgcacgacatcagacagcttcagtgcatgctcaggaacctcacgtgcaacctcacgttccaagaggcttatgatctgacggggaggatacccgggataacggtttgctcaccgaggaagcacgagccgcctcggtgtttaaactatctcacttctcctcacgttgttatatggagcgcggtgactgcttcttgcgcattccctggtttgttcgaagctcaggagcttatggctaaggataggagtggggagatagtgccgtatcatccgccttttaatttggaaccggaggaaggtggtgataagtcgtctacgaggaggtggagagatgggagtttggaggttgatttgccgatgatgcagcttaaggagctgtttaatgttaatcattttattgtgagccaggctaatcctcacattgctccgttgctgcgtttgaaggatatagttagagcttatggtggtagattcgctgctaagctagcgcatctagtggagatggaggtgaaacatagatgcaaccaggtactagagcttggctttccactcggtggactagcaaagctgtttgctcaggagtgggaaggcgatgtaacagttgtgatgcctgctactcttgctcagtactcaaagatcatacagaaccctactcatgtagagctacaaaaagcagctaaccaaggaaggagatgcacttgggagaagctctcagccatcaaagcaaactgcgggatcgaactcgcgcttgacgagtgcgtagccatcctcaaccacatgcggaggctcaagagaagcgccgagagagcctcctcagcttcctcgtctcatcacggattagcgtcaacgacaagattcaatgcttctagaagaatcccatcttggaacgtcatcgctagagagaactcaaccggctcactggacgacctcgtcgctgacagtaataataacaatctccacgcggggaggaacttaagcgacagcgaaactgagagcgtggagatgagttcttggacgaggactggtgggcctttgatgagaacagcttctgctaataggttcactgactttgtccatggtcttgacgtggacattgcgttgacaagagggtttactagcagccctaactctccggcggttcctggtccggttagtccgagctttagtccaagatcgagatccatggcggctccatccgagagcgaatctgacaagagggaaagtagcagtagttcgagtataacagttagtgaaggtgatcttctgcagcctgagagaacgagtaacggtttcgtgttgaacgttgttagaagagagaacttgggaatggctatggggaaccagagtgctgagatgccggagagtgtgcagatagatataccggagagggagatggataatagctctgtctcagaacatgaagatgatgataatgatgatgaagaacataagggctcggttccggttaacgattccggtttacaagaaccgacgtctgctagtgtaatagatgcttag

SEQ ID NO: 47 - protein, 820 aa, Brassica napus
MDISNEANVDPFSIGPTSILGRTIAFRVLFCKSTLQLRRDLFRFLLHWFLTLKLAVSPFVSWFHPRNPQGILAVVTIIAFVLKRYTNVKAKAEMAYRRKFWRNMMRAALTNEEWAHAAKMLDKETPKMNESDLYDEELVKNKLMELRHRRQEGSLRDIMFCMRADLVRNLGNMCNSELHKGRLQVPRHIKEYIDEVSTQLRMVCNNSDSLEDLSLDEKLSFMHETRHAFGRTALLLSGGASLGAFHVGVVRTLVEHKLLPRIIAGSSVGSIICSVVASRSWPELQSFFENSLHSLQFFDQLGSVFTIVKRVMTQGALHDIRQLQCMLRNLTCNLTFQEAYDLTGRIPGITVCSPRKHEPPRCLNYLTSPHVVIWSAVTASCAFPGLFEAQELMAKDRSGEIVPYHPPFNLEPEEGGDKSSTRRWRDGSLEVDLPMMQLKELFNVNHFIVSQANPHIAPLLRLKDIVRAYGGRFAAKLAHLVEMEVKHRCNQVLELGFPLGGLAKLFAQEWEGDVTVVMPATLAQYSKIIQNPTHVELQKAANQGRRCTWEKLSAIKANCGIELALDECVAILNHMRRLKRSAERASSASSSHHGLASTTRFNASRRIPSWNVIARENSTGSLDDLVADSNNNNLHAGRNLSDSETESVEMSSWTRTGGPLMRTASANRFTDFVHGLDVDIALTRGFTSSPNSPAVPGPVSPSFSPRSRSMAAPSESESDKRESSSSSSITVSEGDLLQPERTSNGFVLNVVRRENLGMAMGNQSAEMPESVQIDIPEREMDNSSVSEHEDDDNDDEEHKGSVPVNDSGLQEPTSASVIDA

SEQ ID NO: 48 - DNA, 1023 bp, Brassica napus
atggaggtgaaacatagatgcaaccaagtactagagcttggctttccactcggtggactcgctaagctatttgctcaggagtgggaaggtgatgtaacagttgtgatgcctgctactcttgctcagtactctaagatcatacagaaccctacacacgtggagctacaaaaggcagctaaccaaggaaggagatgcacttgggagaagctctcagccatcaaagcaaactgcgggatcgagcttgcgcttgacgagtgcgtagccattctcaaccacatgcggaggctcaagagaagcgcagagagagcctccacagcttcctcgtctcatcacggattagcttcaacgacaagattcaatgcttctagaagaatcccgtcttggaacgtcatcgctagagagaactcaactggctcactggacgacctcgttactgacagtaacaataataatctccacgcggggaggaacctaagcgacagcgaaacggagagcgtggagatgagttcttggactaggactggtggaccgttgatgagaacggcttctgctaataggttcactgactttgtccatggtcttgacgtggacattgcgttgacaagagggtttactagcagccctaactctccagcggttcctggcccggttagtccgagttttagtccgagatcgagatccttggcggctcaatccgagagcgaatctgacaagagggaaagtagcaacagttcgagtataacagttagtgaaggtgatcttctacagcctgagagaacgagtaacggttttgttttaaacgttgttagaagagagaacttggggatgcctgtggggaaccagagcggtgagctgccggagagtgtacagatagatatacctgagagggagatggataatagctctgtctcagaacatgaagatgataatgatgatgatgatgatgaagaagaagaacataagggctcggttccggttaaagattccggtttacaagattcttgtagtgtaatagatgcttag

SEQ ID NO: 49 - protein, 340 aa, Brassica napus
MEVKHRCNQVLELGFPLGGLAKLFAQEWEGDVTVVMPATLAQYSKIIQNPTHVELQKAANQGRRCTWEKLSAIKANCGIELALDECVAILNHMRRLKRSAERASTASSSHHGLASTTRFNASRRIPSWNVIARENSTGSLDDLVTDSNNNNLHAGRNLSDSETESVEMSSWTRTGGPLMRTASANRFTDFVHGLDVDIALTRGFTSSPNSPAVPGPVSPSFSPRSRSLAAQSESESDKRESSNSSSITVSEGDLLQPERTSNGFVLNVVRRENLGMPVGNQSGELPESVQIDIPEREMDNSSVSEHEDDNDDDDDEEEEHKGSVPVKDSGLQDSCSVIDA

SEQ ID NO: 50 - DNA, 2421 bp, Brassica napus
atggatcatataagcaacgaggccaatgtcgatccattttcaatcggaccaacatccctcatcggccgaaccatcgccttcagagtcctcttctgcaaatcaatgctccagctcagacgcgacctcttccgcttcttgcttcattgcttccttacactcaagccttccgtctcaccctttgtatcctggttccacccccggaacccgcaagggatcttagcagtggtcacaatcatcgccttcgtgttgaaacgttacaccaacgtgaagaagaaggcggagattgtttactggaggaacttctggaggaacacgatgcgcgcggctttgacctacgaggaatggtctcacgccgctaagatgctggagaaggagacgccgaagatgaacgaatccgatctctacgacgaagagttggttaaaaacaagcttcgggagctttgtcatcgccgccgagaaggttgtcttagagacattatgttctgtatgagagctgatctcgtcaggaacctcggtaacatgtgtaactcggagctgcatcaagggaggcttcaggttccgaggcttattaaagagtacatcaacgaggtctctactcagttgagaatggtttgctcctctgagtcattagaggagctttctttagaagagaagctttcttttatgcacgagacacgtcacgcctttggtagaacggctttgcttcttagcggtggagcttctctaggtgcgtttcatgtaggtgtggttaggactctggttgagcataagctcttacctcggattattgctggctctagtgttggctccataatctgttcaatcgtagcaacgaggtcttggcccgagctacagagtttctttgagaactctttgcactctctacagttctttgatcagctaggaagcgttttcacgatcgttaaaagagtcatgacgcaaggcgcgttacacgatataagacagttgcaatgcatgcttaggaacctcacgtgcaacctcacgttccaagaggcttatgatataacagggaggatcctagcgataacggtttgctccccaaggaagcacgaaccgcctcggtgtcttaactatttgacttcgcctcatgtggttatatggagtgcggtgactgcttcttgtgcctttcctggtctctttgaagctcaagaactgatggctaaagatagaagtggagagattgtgccgtatcatccaccttttaatttggatcctgaagagggtactgaaccatatgcacgccggtggagggatggtagtttggaggttgatttgccgatgatgcagcttaaggagctgttcaatgttaatcattttattgtgagccaagctaatcctcacattgtcccgttactgcgtataaaggatatagttcgagcttatggtggtagatttgcagcaaagcttgcccatcttgtggagatggaggtgaaacataggtgcaaccaggtactagaacttggctttcctctcggtggactcgcaaagctttttgctcaggagtgggaaggtgatgtaacagttgtaatgccagctactcttgctcagtactcaaagattatacaaaaccctactcatgtcgagcttcaaaaagccgctaaccaaggaaggagatgcacttgggagaagctatcagccataaaagcaaactgtgggatcgagcttgcgcttgatgagtgtgtaactaatcttaaccatatgcgtaggctcaacagaagcgctgagagagccgctgctgctgctggcacgtcctcctcgtctcatcacggattagcttcagccacaagattcaatggctctagaagaatcccatcttggaacgtcattgctagagagaactcgactggttcactcgatgaactagtcgctgacagtaatctccacgcgtcttcgggctggaacttgagcgatggtgaaactgagagcgtggagttgagttcgtggacaagaaccggtggacctttgatgagaacagcgtctgctaataagttcattgacttcgttcagagtcttgatgtggacattgcattgaccagaggatttagtagcagtcccacttctccggtggttcctggcgtatttagcccaagctttagtccagactcaagatccatggacaacaataacaacatgcttcgaacaaacacttcaagcataatagttactgaaggtgatcttctacagactgagagaaccagtaacggttttgtgttgaacgttgttagaagagaggacttgggaatgtctattgggaatcagaacactgagctgccagagagtgtacagctcgatatacctgacaaggagatggatgatagctctgtatcagaacatgaagtagaccaagaagatgatgatgatgaagaacatggcggctcggacccggttaacgtttcttga

SEQ ID NO: 51 - protein, 806 aa, Brassica napus
MDHISNEANVDPFSIGPTSLIGRTIAFRVLFCKSMLQLRRDLFRFLLHCFLTLKPSVSPFVSWFHPRNPQGILAVVTIIAFVLKRYTNVKKKAEIVYWRNFWRNTMRAALTYEEWSHAAKMLEKETPKMNESDLYDEELVKNKLRELCHRRREGCLRDIMFCMRADLVRNLGNMCNSELHQGRLQVPRLIKEYINEVSTQLRMVCSSESLEELSLEEKLSFMHETRHAFGRTALLLSGGASLGAFHVGVVRTLVEHKLLPRIIAGSSVGSIICSIVATRSWPELQSFFENSLHSLQFFDQLGSVFTIVKRVMTQGALHDIRQLQCMLRNLTCNLTFQEAYDITGRILAITVCSPRKHEPPRCLNYLTSPHVVIWSAVTASCAFPGLFEAQELMAKDRSGEIVPYHPPFNLDPEEGTEPYARRWRDGSLEVDLPMMQLKELFNVNHFIVSQANPHIVPLLRIKDIVRAYGGRFAAKLAHLVEMEVKHRCNQVLELGFPLGGLAKLFAQEWEGDVTVVMPATLAQYSKIIQNPTHVELQKAANQGRRCTWEKLSAIKANCGIELALDECVTNLNHMRRLNRSAERAAAAAGTSSSSHHGLASATRFNGSRRIPSWNVIARENSTGSLDELVADSNLHASSGWNLSDGETESVELSSWTRTGGPLMRTASANKFIDFVQSLDVDIALTRGFSSSPTSPVVPGVFSPSFSPDSRSMDNNNNMLRTNTSSIIVTEGDLLQTERTSNGFVLNVVRREDLGMSIGNQNTELPESVQLDIPDKEMDDSSVSEHEVDQEDDDDEEHGGSDPVNVS

SEQ ID NO: 52 - DNA, 2358 bp, Brassica napus
atggatataagcaacgaggcaagcgtggatccgttctcaataggacccacgggtatcatcggaagaacaatagcctttcgtgtcctgctctgcaaatcaacgactacgctcagacacaaactgttcagattcctcgtgtgtttctacggaggagctagagcttttctatcaccgtgtgtgtcctggctacacccacggaacccacaagggatattagctatggtgacgacgatggcctttatgttgaaccgctacacaagcttaaaagcaaaggctgagatggcttacaggagaaaattctggaggaacatgatgacagctgcattgactcatgaggaatggtctcacgccgccaagatgcttgataaagagactccgaagatgaacgagacagatcttttcgatgtggagctcgtgaggaacaagctcgaggagcttaagcatagacgtcacgagaattctctgagagacattatcttttgcatgagagctgatcttgtgaggaacctcggtaacatgtgcaaccctgagcttcacaaaggaaggcttcaagttccgaaactcatcaaggagtatatagacgaggtctctacgcagcttaggatggtttgtgatacggacactgaagagctgtctctcgaggagaaactatctttcatgcacgagacgaggcacgcgtttggaagaacggctctgcttctaagcggaggagcttctctcggtgcgttccatctcggtgtggttaagacccttgtggaacacaagctgttgccgaggattatagccggttcaagcgtgggttccgtgatgtgtgcggtcgtggggacaaggtcgtggcctgagttacaaagcttctttgaaggctcgtggcacgcgttacagttctttgatcagatgggagggatcttcactattgcgaaacgtgtgatgactcaaggtgcggtccatgagatcagacatttgcaatggaagctgaggaacctcaccaacaatctcacgttccaagaggcttacgacatgacaggacggattcttgggataacagtttgttccccgaggaagcacgagccgcctagatgtcttaactatctgacttctcctcatgtcgtgatatggagtgcagtgactgcttcttgtgccttccctggcctttttgaagctcaggaactcatggctaaagacagaaccggtgagatcgttccttatcacccactttttaatcctgaagagacctcaggagcatcggttaggcgatggagagacggtagtttggagatggacttaccgatgatacaactcagagagctttttaatgttaaccatttcattgtgagtcaagctaaccctcacatcgcacccttcttgaggatgaaggagtttgtgagagcttgtgggggcagatttgcagcaaagcttgctcaacttatggagatggaagtgaagcatagatgtaatcaagtactagagctcgggttgcctctgagagaagtcgctaagctgtttgctcaagagtgggaaggagatgtcacaatcgtcatgcccgccactatttctcagtacttgaagatcatacagaacccatccaacgtagagattcaaaaggcagcaaaccaaggaaggagatgcacctgggagaagctcgccgtcatcaaagcaaacttcggcatcgaactcgccctcgacgagtgcgtcgccgtcctcaaccacatgcgccgcatcaaacgcaccgcacaaagagcagcagccgccgcatccacctccgtctccgtctcaacctcctcccaacatcctctacccgtaggacccaccagattcagcgcctccagaagaatcccttcctggaactgcatcgcgcgcgagaactcagccggatccgtcgacgacgacgtcctcgccgaagcggcgttgctctaccagcaactcgttgtccaccgaggcggtaactgcaacagctcgcacgacggagattcgccggaggctggagactggactaggtcgggcggaccgttggagaggacgacgtctgctcagatgttcatggattacgttcagaatctcgacggtgctgatccgggcgtcgatttgaacggggacgcttcttcgaggagcatcacggtggcggaaggggattatctgcagacggggaggacgcataatggattcgtgttgaatctcgtcagaggggagagtttgaggaggaataatcaagatgtggaggataggcaaaacgttgatgaagctcccgagtgcgtgcaacttgattctccggagaaggatatcgatagaggtagctcggcttcggaagaaggagacgcggaggtagagacggtggtggatacgaatgtacgccaggactaa

SEQ ID NO: 53 - protein, 785 aa, Brassica napus
MDISNEASVDPFSIGPTGIIGRTIAFRVLLCKSTTTLRHKLFRFLVCFYGGARAFLSPCVSWLHPRNPQGILAMVTTMAFMLNRYTSLKAKAEMAYRRKFWRNMMTAALTHEEWSHAAKMLDKETPKMNETDLFDVELVRNKLEELKHRRHENSLRDIIFCMRADLVRNLGNMCNPELHKGRLQVPKLIKEYIDEVSTQLRMVCDTDTEELSLEEKLSFMHETRHAFGRTALLLSGGASLGAFHLGVVKTLVEHKLLPRIIAGSSVGSVMCAVVGTRSWPELQSFFEGSWHALQFFDQMGGIFTIAKRVMTQGAVHEIRHLQWKLRNLTNNLTFQEAYDMTGRILGITVCSPRKHEPPRCLNYLTSPHVVIWSAVTASCAFPGLFEAQELMAKDRTGEIVPYHPLFNPEETSGASVRRWRDGSLEMDLPMIQLRELFNVNHFIVSQANPHIAPFLRMKEFVRACGGRFAAKLAQLMEMEVKHRCNQVLELGLPLREVAKLFAQEWEGDVTIVMPATISQYLKIIQNPSNVEIQKAANQGRRCTWEKLAVIKANFGIELALDECVAVLNHMRRIKRTAQRAAAAASTSVSVSTSSQHPLPVGPTRFSASRRIPSWNCIARENSAGSVDDDVLAEAALLYQQLVVHRGGNCNSSHDGDSPEAGDWTRSGGPLERTTSAQMFMDYVQNLDGADPGVDLNGDASSRSITVAEGDYLQTGRTHNGFVLNLVRGESLRRNNQDVEDRQNVDEAPECVQLDSPEKDIDRGSSASEEGDAEVETVVDTNVRQD

SEQ ID NO: 54 - DNA, 2361 bp, Brassica napus
atggatataagcaacgaggcaagcgtggatccgttctcaataggacccacgggtatcatcggaagaacaatagcctttcgtgtcctgctctgcaaatcaacgactacgctcagacacaaactgttcagattcctcgtgtgtttctacggaggagctagatcttttctatcaccgtgtgtgtcctggctacacccaaggaacccacaagggatattagctatggtgacgacgatggcctttatgttgaaccgctacacaagcttaaaagcaaaggctgagatggcttacaggaggaagttctggaggaacatgatgacagctgcattgactcatgaggaatggtctcacgccgccaagatgcttgataaagagaccccgaagatgaacgagaccgatcttttcgatgtggagctcgtgaggaacaagctcgaggagcttaagcatagacgtcacgagaattctctgagagacattatcttctgcatgagagctgatcttgtgaggaacctcggtaacatgtgtaaccctgagcttcacaaaggaaggcttcaagttccgaaactcatcaaggagtatatagacgaggtctccacgcagcttaggatggtttgtgatacggacactgaagagctttctctcgaggagaaactatctttcatgcacgagacgaggcacgcgtttggaagaacggctttgcttctaagcggaggagcttctctcggcgcgttccatctcggtgtggttaagacccttgtggaacacaagctgttgccgaggattatagccggttcaagcgtggggtcggtgatgtgtgcggtcgtggggacaaggtcgtggcctgagttacaaagcttctttgaaggctcgtggcacgcgttgcagttctttgatcagatgggagggatcttcactattgcgaaacgtgtgatgactcaaggtgcggttcatgagatcagacatttgcaatggaagctgaggaacctcaccaacaatctcacgttccaagaggcttacgacatgacaggacggattcttgggataacggtttgttccccgaggaagcacgagccgcctagatgccttaactatctgacttctcctcatgtggtgatatggagtgcagtgactgcatcttgtgcctttccaggtctttttgaggctcaggaactcatggctaaagacagaactggtgagattgttccttatcacccaatatttaatcctgaagagacctcaggagcatcggttaggcgatggagagacggtagtttggagatggacttaccgatgattcaactcaaagagctttttaatgttaaccatttcattgtgagtcaagctaaccctcacattgcacctttcttgaggatgaaggagtttgtgagagcttgtggaggcagatttgcagcaaagcttgctcaacttatggagatggaagtgaagcatagatgtaatcaagtactagagctcgggttgcctctgagagaagtagctaagctatttgctcaagagtgggaaggggatgtcacaatcgtcatgcccgccactatttctcagtacttgaagatcatacaaaacccatccaacgtagagatccaaaaggcagcaaaccaaggaaggagatgcacctgggagaagctcgccgtcatcaaagcaaacttcggcatcgaactcgccctcgacgagtgcgtcgccgtcctcaaccacatgcgccgcatcaaacgcaccgcacgaagagcagcagccgccgcctccgcctccgtctccgtctcatcctcctcccaacatcctctacccgtaggccccaccagattcagcgcctccagaagaatcccttcctggaactgcatcgcgcgcgagaactcagccggatccgtcgacgacgacgtcctcgccgaagccgcgttgctctaccagcaactcgtcgtccaccgaggcggtaactgcaacagctcgcacgacggagattccccggaggctggagactggactaggtctggcggaccgttggagaggaccacgtctgctcagatgttcatggactacgttcagaatctcgacggtgctgatccgggggttgatttgaacggggacgcttcgtcgaggagcatcacggtggcggaaggggattatctgcagacggggaggacgcataatggattcgtgttgaatcttgttagaggggagagtttgaggaggaataatcaagatgtggaggataggcaaaacgttgatgaagctcccgagtgcgttcagcttgattctccggagaaggatattgatagaggtagctcggcgtctgaagacggagacgcggaggtagagacggtggtggatacgaacctaagccaccacgagtaa

SEQ ID NO: 55 - protein, 786 aa, Brassica napus
MDISNEASVDPFSIGPTGIIGRTIAFRVLLCKSTTTLRHKLFRFLVCFYGGARSFLSPCVSWLHPRNPQGILAMVTTMAFMLNRYTSLKAKAEMAYRRKFWRNMMTAALTHEEWSHAAKMLDKETPKMNETDLFDVELVRNKLEELKHRRHENSLRDIIFCMRADLVRNLGNMCNPELHKGRLQVPKLIKEYIDEVSTQLRMVCDTDTEELSLEEKLSFMHETRHAFGRTALLLSGGASLGAFHLGVVKTLVEHKLLPRIIAGSSVGSVMCAVVGTRSWPELQSFFEGSWHALQFFDQMGGIFTIAKRVMTQGAVHEIRHLQWKLRNLTNNLTFQEAYDMTGRILGITVCSPRKHEPPRCLNYLTSPHVVIWSAVTASCAFPGLFEAQELMAKDRTGEIVPYHPIFNPEETSGASVRRWRDGSLEMDLPMIQLKELFNVNHFIVSQANPHIAPFLRMKEFVRACGGRFAAKLAQLMEMEVKHRCNQVLELGLPLREVAKLFAQEWEGDVTIVMPATISQYLKIIQNPSNVEIQKAANQGRRCTWEKLAVIKANFGIELALDECVAVLNHMRRIKRTARRAAAAASASVSVSSSSQHPLPVGPTRFSASRRIPSWNCIARENSAGSVDDDVLAEAALLYQQLVVHRGGNCNSSHDGDSPEAGDWTRSGGPLERTTSAQMFMDYVQNLDGADPGVDLNGDASSRSITVAEGDYLQTGRTHNGFVLNLVRGESLRRNNQDVEDRQNVDEAPECVQLDSPEKDIDRGSSASEDGDAEVETVVDTNLSHHE

SEQ ID NO: 56 - DNA, 2361 bp, Brassica napus; identical to SEQ ID NO: 54

SEQ ID NO: 57 - protein, 786 aa, Brassica napus; identical to SEQ ID NO: 55

SEQ ID NO: 58 - DNA, 2358 bp, Brassica napus
atggatataagcaacgaggcaagcgtggatccgttctcaataggacccacgggtatcatcggaagaacaatagcctttcgtgtcctgctctgcaaatcaacgactacgctcagacacaaactgttcagattcctcgtgtgtttctacggaggagctagagcttttctatcaccgtgtgtgtcctggctacacccacggaacccacaagggatattagctatggtgacgacgatggcctttatgttgaaccgctacacaagcttaaaagcaaaggctgagatggcttacaggaggaagttctggaggaacatgatgacagctgcattgactcatgaggaatggtctcacgccgccaagatgcttgataaagagaccccgaagatgaacgagaccgatcttttcgatgtggagctcgtgaggaacaagctcgaggagcttaagcatagacgtcacgagaattctctgagagacattatcttttgcatgagagctgatcttgtgaggaacctcggtaacatgtgcaaccctgagcttcacaaaggaaggcttcaagttccgaaactcatcaaggagtatatagacgaggtctctacgcagcttaggatggtttgtgatacggacactgaagagctgtctctcgaggagaaactatctttcatgcacgagacgaggcacgcgtttggaagaacggctctgcttctaagcggaggagcttctctcggtgcgttccatctcggtgtggttaagacccttgtggaacacaagctgttgccgaggattatagccggttcaagcgtgggttccgtgatgtgtgcggtcgtggggacaaggtcgtggcctgagttacaaagcttctttgaaggctcgtggcacgcgttacagttctttgatcagatgggagggatcttcactattgcgaaacgtgtgatgactcaaggtgcggtccatgagatcagacatttgcaatggaagctgaggaacctcaccaacaatctcacgttccaagaggcttacgacatgacaggacggattcttgggataacagtttgttccccgaggaagcacgagccgcctagatgtcttaactatctgacttctcctcatgtcgtgatatggagtgcagtgactgcttcttgtgccttccctggcctttttgaagctcaggaactcatggctaaagacagaaccggtgagatcgttccttatcacccactttttaatcctgaagagacctcaggagcatcggttaggcgatggagagacggtagtttggagatggacttaccgatgatacaactcagagagctttttaatgttaaccatttcattgtgagtcaagctaaccctcacatcgcacccttcttgaggatgaaggagtttgtgagagcttgtgggggcagatttgcagcaaagcttgctcaacttatggagatggaagtgaagcatagatgtaatcaagtactagagctcgggttgcctctgagagaagtcgctaagctgtttgctcaagagtgggaaggagatgtcacaatcgtcatgcccgccactatttctcagtacttgaagatcatacagaacccatccaacgtagagattcaaaaggcagcaaaccaaggaaggagatgcacctgggagaagctcgccgtcatcaaagcaaacttcggcatcgaactcgccctcgacgagtgcgtcgccgtcctcaaccacatgcgccgcatcaaacgcaccgcacgaagagcagcagccgccgcctccgcctccgtctccgtctcatcctcctcccaacatcctctacccgtaggacccaccagattcagcgcctccagaagaatcccttcctggaactgcatcgcgcgcgagaactcagccggatccgtcgacgacgacgtcctcgccgaagcggcgttgctctaccagcaactcgttgtccaccgaggcggtaactgcaacagctcgcacgacggagattcgccggaggctggagactggactaggtcgggcggaccgttggagaggacgacgtctgctcagatgttcatggattacgttcagaatctcgacggtgctgatccgggcgtcgatttgaacggggacgcttcttcgaggagcatcacggtggcggaaggggattatctgcagacggggaggacgcataatggattcgtgttgaatctcgtcagaggggagagtttgaggaggaataatcaagatgtggaggataggcaaaacgttgatgaagctcccgagtgcgtgcaacttgattctccggagaaggatatcgatagaggtagctcggcttcggaagaaggagacgcggaggtagagacggtggtggatacgaatgtacgccaggactaa

SEQ ID NO: 59 - protein, 785 aa, Brassica napus
MDISNEASVDPFSIGPTGIIGRTIAFRVLLCKSTTTLRHKLFRFLVCFYGGARAFLSPCVSWLHPRNPQGILAMVTTMAFMLNRYTSLKAKAEMAYRRKFWRNMMTAALTHEEWSHAAKMLDKETPKMNETDLFDVELVRNKLEELKHRRHENSLRDIIFCMRADLVRNLGNMCNPELHKGRLQVPKLIKEYIDEVSTQLRMVCDTDTEELSLEEKLSFMHETRHAFGRTALLLSGGASLGAFHLGVVKTLVEHKLLPRIIAGSSVGSVMCAVVGTRSWPELQSFFEGSWHALQFFDQMGGIFTIAKRVMTQGAVHEIRHLQWKLRNLTNNLTFQEAYDMTGRILGITVCSPRKHEPPRCLNYLTSPHVVIWSAVTASCAFPGLFEAQELMAKDRTGEIVPYHPLFNPEETSGASVRRWRDGSLEMDLPMIQLRELFNVNHFIVSQANPHIAPFLRMKEFVRACGGRFAAKLAQLMEMEVKHRCNQVLELGLPLREVAKLFAQEWEGDVTIVMPATISQYLKIIQNPSNVEIQKAANQGRRCTWEKLAVIKANFGIELALDECVAVLNHMRRIKRTARRAAAAASASVSVSSSSQHPLPVGPTRFSASRRIPSWNCIARENSAGSVDDDVLAEAALLYQQLVVHRGGNCNSSHDGDSPEAGDWTRSGGPLERTTSAQMFMDYVQNLDGADPGVDLNGDASSRSITVAEGDYLQTGRTHNGFVLNLVRGESLRRNNQDVEDRQNVDEAPECVQLDSPEKDIDRGSSASEEGDAEVETVVDTNVRQD

SEQ ID NO: 60 - protein, 785 aa, Brassica napus; identical to SEQ ID NO: 53

SEQ ID NO: 61 - DNA, 2358 bp, Brassica napus
atggatataagcaacgaggcaagcgtggatccgttctcaataggacccacgggtatcatcggaagaacaatagcctttcgtgtcctgctctgcaaatcaacgactacgctcagacacaaactgttcagattcctcgtgtgtttctacggaggagctagagcttttctatcaccgtgtgtgtcctggctacacccacggaacccacaagggatattagctatggtgacgacgatggcctttatgttgaaccgctacacaagcttaaaagcaaaggctgagatggcttacaggaggaagttctggaggaacatgatgacagctgcattgactcatgaggaatggtctcacgccgccaagatgcttgataaagagaccccgaagatgaacgagaccgatcttttcgatgtggagctcgtgaggaacaagctcgaggagcttaagcatagacgtcacgagaattctctgagagacattatcttttgcatgagagctgatcttgtgaggaacctcggtaacatgtgcaaccctgagcttcacaaaggaaggcttcaagttccgaaactcatcaaggagtatatagacgaggtctctacgcagcttaggatggtttgtgatacggacactgaagagctgtctctcgaggagaaactatctttcatgcacgagacgaggcacgcgtttggaagaacggctctgcttctaagcggaggagcttctctcggtgcgttccatctcggtgtggttaagacccttgtggaacacaagctgttgccgaggattatagccggttcaagcgtgggttccgtgatgtgtgcggtcgtggggacaaggtcgtggcctgagttacaaagcttctttgaaggctcgtggcacgcgttacagttctttgatcagatgggagggatcttcactattgcgaaacgtgtgatgactcaaggtgcggtccatgagatcagacatttgcaatggaagctgaggaacctcaccaacaatctcacgttccaagaggcttacgacatgacaggacggattcttgggataacagtttgttccccgaggaagcacgagccgcctagatgtcttaactatctgacttctcctcatgtcgtgatatggagtgcagtgactgcttcttgtgccttccctggcctttttgaagctcaggaactcatggctaaagacagaaccggtgagatcgttccttatcacccactttttaatcctgaagagacctcaggagcatcggttaggcgatggagagacggtagtttggagatggacttaccgatgatacaactcagagagctttttaatgttaaccatttcattgtgagtcaagctaaccctcacatcgcacccttcttgaggatgaaggagtttgtgagagcttgtgggggcagatttgcagcaaagcttgctcaacttatggagatggaagtgaagcatagatgtaatcaagtactagagctcgggttgcctctgagagaagtcgctaagctgtttgctcaagagtgggaaggagatgtcacaatcgtcatgcccgccactatttctcagtacttgaagatcatacagaacccatccaacgtagagattcaaaaggcagcaaaccaaggaaggagatgcacctgggagaagctcgccgtcatcaaagcaaacttcggcatcgaactcgccctcgacgagtgcgtcgccgtcctcaaccacatgcgccgcatcaaacgcaccgcacaaagagcagcagccgccgcatccacctccgtctccgtctcaacctcctcccaacatcctctacccgtaggacccaccagattcagcgcctccagaagaatcccttcctggaactgcatcgcgcgcgagaactcagccggatccgtcgacgacgacgtcctcgccgaagcggcgttgctctaccagcaactcgttgtccaccgaggcggtaactgcaacagctcgcacgacggagattcgccggaggctggagactggactaggtcgggcggaccgttggagaggacgacgtctgctcagatgttcatggattacgttcagaatctcgacggtgctgatccgggcgtcgatttgaacggggacgcttcttcgaggagcatcacggtggcggaaggggattatctgcagacggggaggacgcataatggattcgtgttgaatctcgtcagaggggagagtttgaggaggaataatcaagatgtggaggataggcaaaacgttgatgaagctcccgagtgcgtgcaacttgattctccggagaaggatatcgatagaggtagctcggcttcggaagaaggagacgcggaggtagagacggtggtggatacgaatgtacgccaggactaa

SEQ ID NO: 62 - DNA, 783 bp, Brassica napus
atgatgagaaagagagaaagtagtaaggtgaagaaagaggagttaaacagaggggcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatctcaaaccaaccaacagaaaagtcgaaaacattccaacaacaacaacatgaataaagtatgtgttatacgtccaaaggcgattaggatcccaaaggctctgacatttcagaatcagagtagtattggtagtaccagtcttcttactgtgaaggaaaacgtgattgatcatcaagctggttctccttcgttgttgggagatcttaaaatcgattttgataaaattcagtctgagtatctcttctctgatttaatgggctttgatggtttgggttgtggaaacgtaatgtctcttgtttcatctgacgaggtgctaggagattatgtttcggctgatgcttcttgtctgggtaatcttgatcttaatagacctttcacttcttgtcttcaagaagattgtctctgggactttaattgttag

SEQ ID NO: 63 - protein, 260 aa, Brassica napus
MMRKRESSKVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVCVIRPKAIRIPKALTFQNQSSIGSTSLLTVKENVIDHQAGSPSLLGDLKIDFDKIQSEYLFSDLMGFDGLGCGNVMSLVSSDEVLGDYVSADASCLGNLDLNRPFTSCLQEDCLWDFNC

SEQ ID NO: 64 - DNA, 783 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatcacaaaccaaccaacagaaaagtcgaaaacattccaacaacaacaacatgaataaagtatctgttatacgtccaaaggcgattaggtgcccaaaggctctgacatttcagaagcagagtagtattggtagtaccagtcttcttactgtgaaggaaaacgtgattgatcaacaagctagttctccttcattgttgggagatcttaaaatcgattttgataaaattcagtctgagtatctcttctctgatctgatggactttgatggtttgggttgtggaaacgtaatgtctcttgtttcatctgacgaggtgctgggagattatgtttcggctgatgcttcttgtctgggtaatcttgatcttaatagacctttcacttcttgtcttcaagaagattgtctctgggactttaattgttag

SEQ ID NO: 65 - protein, 260 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVSVIRPKAIRCPKALTFQKQSSIGSTSLLTVKENVIDQQASSPSLLGDLKIDFDKIQSEYLFSDLMDFDGLGCGNVMSLVSSDEVLGDYVSADASCLGNLDLNRPFTSCLQEDCLWDFNC

SEQ ID NO: 66 - DNA, 828 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatcacaaaccaaccaacagaaaagtcgaaaacattccaacaacaacaacatgaataaagtatctgttatacgtccaaaggcgattaggtgcccaaaggctctgacatttcagaagcagagtagtattggtagtaccagtcttcttactgtgaaggaaaacgtgattgatcaacaagctagttctccttcattgttgggagatcttaaaatcgattttgataaaattcagtctgagtatctcttctctgatctgatggactttgatggtttgggttgtggaaacgtaatgtctcttgtttcatctgacgaggtgctgggagattatgtttcggctgatgcttcttgtctgggtctgcaaatatttatcacaattcgagtattatggaagtgctttttttgtctttcaaacatatataatatgattataagatatcgcaaatacgcaatggcgaaatacattttttga

SEQ ID NO: 67 - protein, 275 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVSVIRPKAIRCPKALTFQKQSSIGSTSLLTVKENVIDQQASSPSLLGDLKIDFDKIQSEYLFSDLMDFDGLGCGNVMSLVSSDEVLGDYVSADASCLGLQIFITIRVLWKCFFCLSNIYNMIIRYRKYAMAKYIF

SEQ ID NO: 68 - DNA, 783 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatcacaaaccaaccaacagaaaagtcgaaaacattccaacaacaacaacatgaataaagtatgtgttatacgtccaaaggcgattaggtgcccaaaggctctgacatttcagaagcagagtagtattggtagtaccagtcttcttactgtgaaggaaaacgtgattgatcaacaagctagttctccttcattgttgggagatcttaaaatcgattttgataaaattcagtctgagtatctcttctctgatctgatggactttgatggtttgggttgtggaaacgtaatgtctcttgtttcatctgacgaggtgctgggagattatgtttcggctgatgcttcttgtctgggtaatcttgatcttaatagacctttcacttcttgtcttcaagaagattgtctctgggactttaattgttag

SEQ ID NO: 69 - protein, 260 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVCVIRPKAIRCPKALTFQKQSSIGSTSLLTVKENVIDQQASSPSLLGDLKIDFDKIQSEYLFSDLMDFDGLGCGNVMSLVSSDEVLGDYVSADASCLGNLDLNRPFTSCLQEDCLWDFNC

SEQ ID NO: 70 - DNA, 828 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatcacaaaccaaccaacagaaaagtcgaaaacattccaacaacaacaacatgaataaagtatgtgttatacgtccaaaggcgattaggtgcccaaaggctctgacatttcagaagcagagtagtattggtagtaccagtcttcttactgtgaaggaaaacgtgattgatcaacaagctagttctccttcattgttgggagatcttaaaatcgattttgataaaattcagtctgagtatctcttctctgatctgatggactttgatggtttgggttgtggaaacgtaatgtctcttgtttcatctgacgaggtgctgggagattatgtttcggctgatgcttcttgtctgggtctgcaaatatttatcacaattcgagtattatggaagtgctttttttgtctttcaaacatatataatatgattataagatatcgcaaatacgcaatggcgaaatacattttttga

SEQ ID NO: 71 - protein, 275 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVCVIRPKAIRCPKALTFQKQSSIGSTSLLTVKENVIDQQASSPSLLGDLKIDFDKIQSEYLFSDLMDFDGLGCGNVMSLVSSDEVLGDYVSADASCLGLQIFITIRVLWKCFFCLSNIYNMIIRYRKYAMAKYIF

SEQ ID NO: 72 - DNA, 783 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatcacaaaccaaccaacagaaaagtcgaaaacattccaacaacaacaacatgaataaagtatctgttatacgtccaaaggcgattaggtgcccaaaggctctgacatttcagaagcagagtagtattggtagtaccagtcttcttactgtgaaggaaaacgtgattgatcaacaagctagttctccttcatcgttgggagatcttaaaatcgattttgataaaattcagtctgagtatctcttctctgatctgatggactttgatggtttgggttgtggaaacgtaatgtctcttgtttcatctgacgaggtgctgggagattatgtttcggctgatgcttcttgtctgggtaatcttgatcttaatagacctttcacttcttgtcttcaagaagattgtctctgggactttaattgttag

SEQ ID NO: 73 - protein, 260 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVSVIRPKAIRCPKALTFQKQSSIGSTSLLTVKENVIDQQASSPSSLGDLKIDFDKIQSEYLFSDLMDFDGLGCGNVMSLVSSDEVLGDYVSADASCLGNLDLNRPFTSCLQEDCLWDFNC

SEQ ID NO: 74 - DNA, 828 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaagacttccaaaatcacaaaccaaccaacagaaaagtcgaaaacattccaacaacaacaacatgaataaagtatctgttatacgtccaaaggcgattaggtgcccaaaggctctgacatttcagaagcagagtagtattggtagtaccagtcttcttactgtgaaggaaaacgtgattgatcaacaagctagttctccttcatcgttgggagatcttaaaatcgattttgataaaattcagtctgagtatctcttctctgatctgatggactttgatggtttgggttgtggaaacgtaatgtctcttgtttcatctgacgaggtgctgggagattatgtttcggctgatgcttcttgtctgggtctgcaaatatttatcacaattcgagtattatggaagtgctttttttgtctttcaaacatatataatatgattataagatatcgcaaatacgcaatggcgaaatacattttttga

SEQ ID NO: 75 - protein, 275 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKRLPKSQTNQQKSRKHSNNNNMNKVSVIRPKAIRCPKALTFQKQSSIGSTSLLTVKENVIDQQASSPSSLGDLKIDFDKIQSEYLFSDLMDFDGLGCGNVMSLVSSDEVLGDYVSADASCLGLQIFITIRVLWKCFFCLSNIYNMIIRYRKYAMAKYIF

SEQ ID NO: 76 - DNA, 426 bp, Brassica napus
atgatgagaaagagagaaagtagtagggtgaagaaagaggagttaaacagaggagcttggaccgatcaagaagacaagatccttaaagactatatcatgttccacggcgaaggaaaatggagcacactcccaaaccaagctggtctcaagaggtgtggcaaaagctgcagacttcggtggaagaactacttgagaccaggcataaagcgcggaaacatctcatctgatgaagaagaacttataatccgcctccataatctccttggaaacagatggtcgttgatagctgggaggcttccagggcgaacagacaatgaaataaagaaccactggaactcaaacctccgcaaaacaataccgaaaacatatctaaaatactcgaatatacttaatatacacaaaacaattcaggtacttgggtattag

SEQ ID NO: 77 - protein, 141 aa, Brassica napus
MMRKRESSRVKKEELNRGAWTDQEDKILKDYIMFHGEGKWSTLPNQAGLKRCGKSCRLRWKNYLRPGIKRGNISSDEEELIIRLHNLLGNRWSLIAGRLPGRTDNEIKNHWNSNLRKTIPKTYLKYSNILNIHKTIQVLGY

SEQ ID NO: 78 - DNA, 831 bp, Camelina sativa
atggcgtcttctgcagctctcggatctcttcatcagactttagggtcacagagtgagcttcatttgctttctggaaactggtctgcctctggtacttcttgcgttccacggtggagattatccaacaggagtagcaattacacgcttgtgttacgtgcaaaggcctctaaaacttcgacaacaaccaaaagcgatgattcatctgatgcgactgtgtcaaacgggaagaaatctgttcgacggacaaccttcccgaaagaagtggaggcactggttcacgagatgtgtgatgagactgaggttgctgtcctgaaacttaaggttggagatttcgagatgaacctaaaacggaagattggagcggccacaaaccccattcctgtggaggatatatctccaaccgtagcacctccgattccttctgagcccatggataaatctgtttcttctgctcccagcccatctaaagcaaaaccgtctgaaaaagtatctccatttatgaatacatcatatgggaaaccagcgaagttggtagctttggaggcatctggatcaaacaattatgttctagtcaaatctccctcagttggcgagtttcacagaagcagaactgtaaaaggaaagaaactatctcctagctgcaaagagggtgatgaaataaaggaaggccaagttattggatacttacatcagttgggaacagaacttccagtgacgtcggatgtagctggggaagtcctcaagcttctttcagatgacggagactccgtaggttatggtgatcctctggttgcggtcttgccatcgttccacgatatcaacatccagtga

SEQ ID NO: 79 - DNA, 831 bp, Camelina sativa
atggcgtcttctgcagctctcggatctcttcatcagactttagggtcacagagtgagcttcacttgctttctggaaattggtctgcttctggtacttcttgtgtaccacggtggagattatccaacaggagcagcaattacacgcttgtgttacgtgcaaaggcctctaaaacttcgacaacaaccaaaagcgatgattcatctgatgcgactgtgtcaaacgggaagaaatctgttcgacggacaactttcccgaaagaagtggaggcactggttcacgagatgtgtgatgagactgaggttgctgtcctgaaacttaaggttggagatttcgagatgaacctaaaacggaagattgaagcggccacaaaccccattcctgtggaggatatatctccaaccgtagcacctccgattccttctgagcccatgaatcaatcggtttcctctattcctagcccatctaaagcaaaaccttctgaaaaagtatctccatttataaatacatcatatgggaaaccagcaaagttggcagctttggaggcatctggatcaaataattatgttctagtcaaatctccctcagttggcgagtttcacagaagcagaactgtaaaaggaaagaaactatctcctagctgcaaagagggtgatgaaataaaggaagggcaagttattggatacttacatcagttgggaacagaacttccagtgacgtcggatgtagctggggaagtcctcaagcttctttcagatgacggagactccgtaggttatggtgatcctctggttgcggtcttgccatcgttccacgatatcaacatccagtga

SEQ ID NO: 80 - DNA, 831 bp, Camelina sativa
atggcgtcttctgcagctctcggatctcttcatcagactttagggtcacagagtgagcttcacttgctttctggaaattggtctgcttctggtacttcttgtgtaccacggtggagattatccaacaggagcagcaattacacgcttgtgttacgtgcaaaggcctctaaaacttcgacaacaaccaaaagcgatgattcatctgatgcaactgtgtcaaacgggaagaaatctgttcgaaggacaactttcccgaaagaagtggagacactggttcacgagatgtgtgatgagactgaggttgctgtcctgaaactcaaggttggagatttcgagatgaacctaaaacggaagattggagctaccacaaaccccattcctgtggaggatatatctccaaccgtagcacctccaattccttctgagcccatgaatcaatcggtttcctctgctcccagcccatctacagcaaaaccgtctgaaaaagtatctccatttatgaatacatcatatgggaaaccagcaaagttggcagctttggaggcatctggatcaaacaattatgttctagtcaaatctccctcagttggcgagtttcacagaagcagaactgtaaaaggaaagaaactatctcctagctgcaaagagggtgatgaaataaaggaaggccaagtgattggatacttacatcagttgggaacagaacttccagtgacgtcggatgtagctggggaagtcctcaagcttctttcagatgacggagactccataggttatggtgatcctctggttgcggtcttgccatcgttccacgatatcaacatccagtga

SEQ ID NO: 81 - DNA, 831 bp, Camelina sativa
atgaattcctgtagtttaggagctccaaaagttagaattgctgcagcaaatttcagtagattaagatgtggaaacttgctgatacccaacaatcaaagactttttattgaccaaagccaaagccaaagccccatcaagtatccgagtctgaggacaactctgcgagctgtgaaagctgtccaattgtctactgtcccacctgcagatatagcagctgttgcagacgtagaggattctcaagagaccgaatcaactgttgtgaatactcagctcattcccaagtcctctgaggtggaagcacttatcaaagaaatcacagattcctcatcgattgcagagtttgaactgaaactgggaggtttccgcctatatgtagcaaggaaattagctgaccaaagtagtccaccgcctcagcaaattccacctgtggttgctgcaagctcagcccctgagggggttcatactaatggctcagccacttcctcgtcattggctatcacaaaatcagcatctccatcagacagaccacaaacactcgctaacaaagctgctgatcagggtttagtgattctccaatctccaacggttggttatttcaggagatccaagaccataaaaggcaaacgcactcctacaatctgtaaagagaaagacatagtgaaagaaggtcaagttctatgctacattgaacaactcggtggccagatcccagttgagtctgatgtttccggtgagattgtcaaaatactccgggaagatggcgagcctgtaggatacaacgatgctctcattaccgttcttccttcatttcctggcatcaagaagctttag

SEQ ID NO: 82 - DNA, 810 bp, Camelina sativa
atgaattcctgtactttaggagctccaaaagttcggatttctgcagcaaatttcagtagattaagatgtggaaacttgctgatacccaacaatcaaagactactaattggccaaagccccatcaagtatccgagtctgaggacaactctgcgagctgtccaattgtctactgtcccacctgcagcaatagcagctgttgcagacgtagaggattctgaagagaccgaatcaactgttgtgaatactcagctcattcccaagtcctctgaggtggaagcacttattaaagaaatcacagattcctcatcgattgcagagtttgaactgaaactgggaggtttccgcctatatgtagcaaggaaattagctgaccaaagtagtccaccgcctcagcaaattccacctgtggttgctgcaagctcagctcctgagggggttcatactaatggctcagccacttcctcgtcattggctatcacaaaatcagcatctccatcagacagaccacaaacactcgctaacaaagctgctgatcagggtttagtgattctccaatctccaacggttggttatttcaggagatccaagaccataaaaggcaaacgcactcctacaatctgtaaagagaaagacacagtaaaagaaggtcaagttctatgctacattgaacaactcggtggccagatcccagttgagtctgatgtttccggtgagattgtcaaaatactccgggaagatggcgagcctgtaggatacaacgatgctctcatcaccgttcttccttcatttcctggcatcaagaagctttag

SEQ ID NO: 83 - DNA, 810 bp, Camelina sativa
atgaattcctgtagtttaggagctccaaaagttcggatttctgcagcatatttcagtagattaagatgtggaaacttgctgatacccaacaatcaaagactactaattggccaaagccccatcaagtatcagagtctgaggacaactctgcgagctgtccaattgtctactgtcccacctgcagaaatagcagctgttgcagacgtagaggattctgaagagaccgaatcaactgttgtgaatactcagctcattcccaagtcctctgaggtggaagcacttatcaaagaaatcacagattcctcatcgattgcagagtttgaactgaaactgggaggtttccgcctatatgtagcaaggaaattagctgaccaaagtagtccaccgcctcagcaaattccacctgtggttgctgcaagctcagctcctgagggggttcatactaatggctcagccacttcctcgtcattggctatcacaaaatcagcatctccatcagacagaccacaaacactcgctaacaaagctgctgatcagggtttagtgattctccaatctccaacggttggttatttcaggagatccaagaccataaaaggcaaacgcactcctacaatctgtaaagagaaagacacagtgaaagaaggtcaagttctatgctacattgaacaactcggtggccagatcccagttgagtctgatgtttccggtgagattgtcaaaatactccgggaagatggcgagcctgtaggatacaacgatgctctcattaccgttcttccttcatttcctggcatcaagaagctttag

SEQ ID NO: 84 - DNA, 792 bp, Camelina sativa
atggcttcctgtagcctaggagttcctaaaattaaaatctcagcagtagaccttagtagagtaagttctggaagcttactgataccattcagccaaagatcattgcttggacaaaggccggtgaagtacttgagtctcaggacaacttttggatctgtgaaagctgtccaagtatctactgtcccaaccgcagaaacatcagctactatagaagtagaagattctgaagaaaccaagtcatctccattgaacgctcagctagttcccaagccatctgaggtggaagctcttgtcactgaaatatgcgattcctcatcaattgcagagtttgaattgaaactggggggtttccgcctatatgtagcaagggatctaactgacaaaagtagtccgcagcctcatccagttcctgctgtggctgctgccagtgaaactaccaagagtcctgattcgaatggatcaactccttctacttcattggctatcacaagaccagcatcctcagctgctgatcacggtttgatgattctccaatctccaaaagtagggttcttcaggagatccaaaactataaagggtaaacgcatgccttcgtcatgtaaagagaaagaccaagtgaaagaaggtcaaattctgtgctacattgaacaactcggtggccaattcccaatagagtctgatgtcagcggcgaggttgtcaaaatactccgagaagatggagagcctgtaggatacaatgatgctctcatctcgatccttccctctttccctgggatcaagaagcttcagtaa

SEQ ID NO: 85 - DNA, 792 bp, Camelina sativa
atggcatcctgtagcctaggagttcctaaaattaaaatctcagcagtagaccttagtagagtaagttctggaagcttactgataccattcagtcaaagatcattgcttggacaaaggccggtgaagtacttgagtctgaggacaacttttggatctgtgaaagctgtacaagtatctactgtcccagctgcagaaacatcagctactgtaggagtagaagattctgaagaaaccaagtcatccccattgaacgctcagctagttcccaagcgatctgaggtggaagctcttgtcactgaaatatgcgactcctcatcaattgcagagtttgaactgaaactggggggtttccgcctatatgtagcaagggatctagctgacaaaagtagtccgcagcctcatccaattcctgctgtggctgctgcaagtgaaactaccaagagtcctgattcgaatggatcaacaccttctacttcattggctatcacaagaccagcatcttcagctgctgatcagggtttgatgattctccaatctccaaaagtagggttctttaggagatccaaaaccataaagggtaaacgcatgccttcgtcatgtaaagagaaagaccaagtgaaagaaggtcaaattctgtgctacattgaacaactcggtggccaattcccaatagagtctgatgtcagcggtgaggttgtcaaaatactccgcgaagatggagaacctgtaggatacaatgatgctctcatctcgatccttccctctttccctgggatcaagaagcttcagtaa

SEQ ID NO: 86 - DNA, 792 bp, Camelina sativa
atggcttcctgtagcctaggagttcctaaaattaaaatctcagcagtagaccttagtagagtaagttctggaagcttgctggtaccattcagtcaaagatcattgcttggacaaaggacggtgaagtacttgagtctgaggaaaacttttggatctgtgaaagctgtacaactatctactgtcccagctgcagaaacatcagctactgtaggagtagaagattctgaagaaaccaagtcatctccattgaacgctcagctagttcccaatccatctgaggtggaagctcttgtcactgaaatatgcgactcctcatcaattgcagagtttgaactgaaactggggggtttccgcctatatgtagcaagggatctagctgacaaaagtagtccgcagcctcatccaattcctgctgtggctgctgcaagtgaaactaccaagagtcctgattcgaatggatcaacaccttctacttcattggctatcacaagaccagcatcttcagctgctgatcagggtttgatgattctccaatctccaaaagtagggttctttaggagatccaaaaccataaagggtaaacgcatgccttcgtcatgtaaagagaaagaccaagtgaaagaaggtcaaattctgtgctacattgaacaactcggtggccaattcccaatagagtctgatgtcagcggcgaggttgtcaaaatactccgagaagatggagagcctgtagggtacaatgatgctctcatctcgatccttccctctttccctgggatcaagaagcttcagtaa

SEQ ID NO: 87 - DNA, 2418 bp, Glycine max
atggatcatataagtaacgaggctagtgttgacccttttgcgattggcccttctagcattcttggtagaacaattgctttcagggttctcttctgcaagtctatctcacaattgaggtatcatatactttttgtgctgtcgaatatcttcaaaagatttaggggattttgggcacccattgtgtcatggttgcatccgagaaaccagggcatattggcaatgatgacaatgattgctttcttgttgaaacggtacaccagtgtgaaagtgagggccaatatcgcatataagcggaaattttggagaaatatggtgagagctgcattgacctatgaggagtgggctcatgcagccaagatgcttgatagagagacacaaaagatgaatgaatcagatctttatgatgtggagttggtgagtaacaagcttgaagaacttcgccatcgccggcaagaggggtctcttagagatataatattttgtatgcgtgcagatcttattaggaatctaggtaatatgtgtaaccctgagctccacaaagctaggcttcagatgccaagactaatcaaggagtatattgacgaggtatcaactcagttgagaatggtctgcaactctgattcggaggagcttgcattggaagaaaagctttctttcatgcatgaaacgagacatgcatttgggaggactgctttgttgttaagtgggggtgcttcccttggagcttttcatgttggtgtggttaaaacactggttgaacataaacttttgcctaggataattgctggttcaagtgtaggatccattatgtgttctattgttgccactaggtcttggcctgagcttcaaagcttttttgaggattcattgcactcactacagttttttgatcaaatgggtgggatttttacagttgtgaagagggttacaacatatggcgcagttcatgagatcagacaattgcagatgttgttgaggcatctaacaagcaatcttacatttcaagaagcatatgacatgacaggtcgaattcttgggattacagtttgttccccaagaaagcatgaaccacctagatgtcttaactacttgacttcacctcatgttgtaatatggagcgcagttactgcttcttgtgcctttcctggccttttcgaggctcaggaattgatggcaaaggatagaagtggagagattgttccttaccatcctccatttaatttgggtcctgaaaagggttccacttcagtgcgccgttggagggatggtagcttggagaatgatttacctatgatgcagttgaaagagttgttcaacgtcaatcactttatagttagtcaggccaatcctcatattgcaccattattgagattgaaagaatttgtacgagcttatggaggtaattttgcagcaaagcttgctcatctagcagagatggaggtgaaacatagatgtaatcaggtactggaactcggctttcccttaggtggacttgccaaactgtttgctcaagattgggaaggtgatgtaacagttgttatgcctgcaactcttgctcagtacttaaaaatcatacagaacccttcttatgtagagcttcaaaaggcagctaaccaagggagaagatgcacctgggagaagctttcggccataaaagcaaattgtggaattgagcttgctcttgatgagtctgttgcaattctaaatcatatgaggcgactcaaaagaaatgctgagagagttgctactgcttctcatggtcttcccagtactgtcaaattcagtggttcaagaagaattccatcgtggaacatcattgcacgagagaattctacaggttccctagaagaccttcatgctgatgctgcttcctcattgcatcaaggtgttattagtcccagtggagcaactggtaaaaactggaagtcccatcgtatcattcatgatgcaagtgacagtgaatctgaaactgctgacttgaattcttggaccagatccggtgggcctttgatgagaactacttcagcagatatgttcattgactttgtccaggacttagaatttcataaagttaataccgaactaaatagaggcaaggtgactcacactagtcctcatgattttcagcatcacagttcaaggcacacaacagttaataggtggtctgagagcacagaaaacgatcagaaagaaaatggcaacaggattgtcatgaatgaatctaacataatggtaactgaaggtgatcttttacagcctgaaaggatccataatgggattgtgtttaatgttgtcaagaaagaaaacttgacaccaaataggaatcatgattataatagttataacaatgaagttgctgaatgtgtacaaattgactttccagggaaggaggtggatgctgctagttcagcttcctaa

SEQ ID NO: 88 - DNA, 2277 bp, Glycine max
atggatcatataagtaacgaggctagtgttgacccttttgcgattggcccttctagcattcttggtagaacaattgctttcagggttctcttctgcaagtctatctcacaattgaggtatcatatactttttgtgctgtcgaatatcttcaaaagatttaggggattttgggcacccattgtgtcatggttgcatccgagaaaccagggcatattggcaatgatgacaatgattgctttcttgttgaaacggtacaccagtgtgaaagtgagggccaatatcgcatataagcggaaattttggagaaatatggtgagagctgcattgacctatgaggagtgggctcatgcagccaagatgcttgatagagagacacaaaagatgaatgaatcagatctttatgatgtggagttggtgagtaacaagcttgaagaacttcgccatcgccggcaagaggggtctcttagagatataatattttgtatgcgtgcagatcttattaggaatctaggtaatatgtgtaaccctgagctccacaaagctaggcttcagatgccaagactaatcaaggagtatattgacgaggtatcaactcagttgagaatggtctgcaactctgattcggaggagcttgcattggaagaaaagctttctttcatgcatgaaacgagacatgcatttgggaggactgctttgttgttaagtgggggtgcttcccttggagcttttcatgttggtgtggttaaaacactggttgaacataaacttttgcctaggataattgctggttcaagtgtaggatccattatgtgttctattgttgccactaggtcttggcctgagcttcaaagcttttttgaggattcattgcactcactacagttttttgatcaaatgggtgggatttttacagttgtgaagagggttacaacatatggcgcagttcatgagatcagacaattgcagatgttgttgaggcatctaacaagcaatcttacatttcaagaagcatatgacatgacaggtcgaattcttgggattacagtttgttccccaagaaagcatgaaccacctagatgtcttaactacttgacttcacctcatgttgtaatatggagcgcagttactgcttcttgtgcctttcctggccttttcgaggctcaggaattgatggcaaaggatagaagtggagagattgttccttaccatcctccatttaatttgggtcctgaaaagggttccacttcagtgcgccgttggagggatggtagcttggagaatgatttacctatgatgcagttgaaagagttgttcaacgtcaatcactttatagttagtcaggccaatcctcatattgcaccattattgagattgaaagaatttgtacgagcttatggaggtaattttgcagcaaagcttgctcatctagcagagatggaggtgaaacatagatgtaatcaggtactggaactcggctttcccttaggtggacttgccaaactgtttgctcaagattgggaaggtgatgtaacagttgttatgcctgcaactcttgctcagtacttaaaaatcatacagaacccttcttatgtagagcttcaaaaggcagctaaccaagggagaagatgcacctgggagaagctttcggccataaaagcaaattgtggaattgagcttgctcttgatgagtctgttgcaattctaaatcatatgaggcgactcaaaagaaatgctgagagagttgctactgcttctcatggtcttcccagtactgtcaaattcagtggttcaagaagaattccatcgtggaacatcattgcacgagagaattctacaggttccctagaagaccttcatgctgatgctgcttcctcattgcatcaaggtgttattagtcccagtggagcaactggtaaaaactggaagtcccatcgtatcattcatgatgcaagtgacagtgaatctgaaactgctgacttgaattcttggaccagatccggtgggcctttgatgagaactacttcagcagatatgttcattgactttgtccaggacttagaatttcataaagttaataccgaactaaatagaggcaaggtgactcacactagtcctcatgattttcagcatcacagttcaaggcacacaacagttaataggtggtctgagagcacagaaaacgatcagaaagaaaatggcaacaggattgtcatgaatgaatctaacataatggtaactgaagggaaggaggtggatgctgctagttcagcttcctaa

SEQ ID NO: 89 - DNA, 2400 bp, Glycine max
atggatcatataagtaacgaggctagtgttgacccttttgcgattggcccttctagcattcttggtagaacaattgctttcagggttctcttctgcaagtctatctcacaattgaggtatcatatactttttgtgctgtcgaatatcttcaaaagatttaggggattttgggcacccattgtgtcatggttgcatccgagaaaccagggcatattggcaatgatgacaatgattgctttcttgttgaaacggtacaccagtgtgaaagtgagggccaatatcgcatataagcggaaattttggagaaatatggtgagagctgcattgacctatgaggagtgggctcatgcagccaagatgcttgatagagagacacaaaagatgaatgaatcagatctttatgatgtggagttggtgagtaacaagcttgaagaacttcgccatcgccggcaagaggggtctcttagagatataatattttgtatgcgtgcagatcttattaggaatctaggtaatatgtgtaaccctgagctccacaaagctaggcttcagatgccaagactaatcaaggagtatattgacgaggtatcaactcagttgagaatggtctgcaactctgattcggaggagcttgcattggaagaaaagctttctttcatgcatgaaacgagacatgcatttgggaggactgctttgttgttaagtgggggtgcttcccttggagcttttcatgttggtgtggttaaaacactggttgaacataaacttttgcctaggataattgctggttcaagtgtaggatccattatgtgttctattgttgccactaggtcttggcctgagcttcaaagcttttttgaggattcattgcactcactacagttttttgatcaaatgggtgggatttttacagttgtgaagagggttacaacatatggcgcagttcatgagatcagacaattgcagatgttgttgaggcatctaacaagcaatcttacatttcaagaagcatatgacatgacaggtcgaattcttgggattacagtttgttccccaagaaagcatgaaccacctagatgtcttaactacttgacttcacctcatgttgtaatatggagcgcagttactgcttcttgtgcctttcctggccttttcgaggctcaggaattgatggcaaaggatagaagtggagagattgttccttaccatcctccatttaatttgggtcctgaaaagggttccacttcagtgcgccgttggagggatggtagcttggagaatgatttacctatgatgcagttgaaagagttgttcaacgtcaatcactttatagttagtcaggccaatcctcatattgcaccattattgagattgaaagaatttgtacgagcttatggaggtaattttgcagcaaagcttgctcatctagcagagatggaggtgaaacatagatgtaatcaggtactggaactcggctttcccttaggtggacttgccaaactgtttgctcaagattgggaaggtgatgtaacagttgttatgcctgcaactcttgctcagaacccttcttatgtagagcttcaaaaggcagctaaccaagggagaagatgcacctgggagaagctttcggccataaaagcaaattgtggaattgagcttgctcttgatgagtctgttgcaattctaaatcatatgaggcgactcaaaagaaatgctgagagagttgctactgcttctcatggtcttcccagtactgtcaaattcagtggttcaagaagaattccatcgtggaacatcattgcacgagagaattctacaggttccctagaagaccttcatgctgatgctgcttcctcattgcatcaaggtgttattagtcccagtggagcaactggtaaaaactggaagtcccatcgtatcattcatgatgcaagtgacagtgaatctgaaactgctgacttgaattcttggaccagatccggtgggcctttgatgagaactacttcagcagatatgttcattgactttgtccaggacttagaatttcataaagttaataccgaactaaatagaggcaaggtgactcacactagtcctcatgattttcagcatcacagttcaaggcacacaacagttaataggtggtctgagagcacagaaaacgatcagaaagaaaatggcaacaggattgtcatgaatgaatctaacataatggtaactgaaggtgatcttttacagcctgaaaggatccataatgggattgtgtttaatgttgtcaagaaagaaaacttgacaccaaataggaatcatgattataatagttataacaatgaagttgctgaatgtgtacaaattgactttccagggaaggaggtggatgctgctagttcagcttcctaa

SEQ ID NO: 90 - DNA, 2415 bp, Glycine max
atggatcatataaataacgaggctagtgttgacccttttgcgattggcccttctagcattcttggtagaacaattgctttcagggttctcttctgcaagtctatctcacaattgaggcatcatatacattttgtgttgttggatatcttgaaaagattttggggattttggggacccattgtgtcatggttgcatccaagaaacccgcaggggatattggcaatgatgacaataattgctgtcttgttgaaacgttacagcagtgtgaaggtgagggccgagatagcatataggcagaatttttggagaaatatgatgcgagctgcattgacctatgaggagtgggctcatgcagccaagatgattgatagagagacaccaaagatgaatgaatcaaaagacctttatgatgtggagttggtgagtaacaagcttcaagagcttcgccatcgcaggcaagaggggtctcttagagatataatattttgtatgcgtgcagatcttgttaggaatctaggtaatatgtgtaatccagagctacacaaaggtaggcttcagatgccaagactaatcaaggagtatattgatgaggtatcaactcaattgagaatggtttgcaactctgattcagaggagcttgcattggaagaaaagctttctttcatgcatgaaactagacacgcatttgggaggactgctttgttgttaagtggaggtgcttcccttggagcttttcatgttggtgtggttaaaacactggttgaacataaacttttgcctaggataattgctggttcaagtgtaggatccattatgtgttctattgttgccactaggtcttggcctgagctccaaagcttttttgaggattcattgcactcattacagttttttgatcaaatgggtgggatttttacagttgtgaagagggtaacaacatatggtgcagttcatgagatcagacagttgcaaatgttgttgaggaatctaacaagcaatcttacatttcaagaagcatatgacatgacgggtcgaattctcggaattacagtttgctccccgagaaagcatgaaccacccagatgtcttaactacttgacttcacctcatgttgttatatggagcgcagtcactgcttcttgtgcctttcctggccttttcgaggctcaggaattgatggcaaaggatagaagtggagagattgttccttaccatccaccatttaatttgggtcctgaaaagggttccacttcagtgcgccgttggagggatggtagcttggagatggatttgcctatgatgcaattgaaagagctcttcaatgtcaatcattttatagttagtcaggccaatcctcacattgccccattattgagattcaaagaaattatccgagcttatggaggtaattttgcagcaaagcttgctcatctagcagagatggaggtgaaacatagatgtaatcaagtactggaactcggctttccattaggtggactagccaaactgtttgctcaagactgggagggtgatgtaacagttgttatgcctgcaactcctgcccaatacttaaaaatcatacagaacccttcttatgtagagcttcaaaaggcagctaaccaaggcagaagatgcacctgggagaagctttcggccatgaaagcaaattgtggaattgagcttgctcttgatgagtctgttgcaattctcaatcatatgagacgactcaaaagaaatgctgagagaactgctgctgcttctcatggtcttcccagtacaggcaaatttagtggttcaagaagaattccatcgtggaacatcattgcacgagagaattctacaggctccctagaagaccttcatgctgctgctgctgctacttcctcattgcatcaaggtgttattagtcccagtggagccactggtaaaaattggaagtcccaccgtagctttcatgatgcaagtgacagtgaatctgaaagtgctcatgacttgaattcttggaccagatctggtgggcctttgatgagaactacatcagcagatatgttcattgactttttccagaacatagaagttgataccgaactaaatagaggcaaggtgacaaacactaccactagccctcgtgattttcagcatcacatttcaaggcacacaacacctgatcataataggtggtgctctaagagaacagaaatcgatcagaaagaaaatggcaacaggatgattgccatgaatggatctaacataatggtaactgaagggaaggaggtggatgctcttagttcagcttccgaaaatggcgatgatggttccacaatggccaggtccctaactgaaaaaccagattataattcaacaaatcatttcagcaaagattttggtgaagatcaaagcattgctgacaattaa

SEQ ID NO: 91 - DNA, 2565 bp, Glycine max
atggatcatataaataacgaggctagtgttgacccttttgcgattggcccttctagcattcttggtagaacaattgctttcagggttctcttctgcaagtctatctcacaattgaggcatcatatacattttgtgttgttggatatcttgaaaagattttggggattttggggacccattgtgtcatggttgcatccaagaaacccgcaggggatattggcaatgatgacaataattgctgtcttgttgaaacgttacagcagtgtgaaggtgagggccgagatagcatataggcagaatttttggagaaatatgatgcgagctgcattgacctatgaggagtgggctcatgcagccaagatgattgatagagagacaccaaagatgaatgaatcaaaagacctttatgatgtggagttggtgagtaacaagcttcaagagcttcgccatcgcaggcaagaggggtctcttagagatataatattttgtatgcgtgcagatcttgttaggaatctaggtaatatgtgtaatccagagctacacaaaggtaggcttcagatgccaagactaatcaaggagtatattgatgaggtatcaactcaattgagaatggtttgcaactctgattcagaggagcttgcattggaagaaaagctttctttcatgcatgaaactagacacgcatttgggaggactgctttgttgttaagtggaggtgcttcccttggagcttttcatgttggtgtggttaaaacactggttgaacataaacttttgcctaggataattgctggttcaagtgtaggatccattatgtgttctattgttgccactaggtcttggcctgagctccaaagcttttttgaggattcattgcactcattacagttttttgatcaaatgggtgggatttttacagttgtgaagagggtaacaacatatggtgcagttcatgagatcagacagttgcaaatgttgttgaggaatctaacaagcaatcttacatttcaagaagcatatgacatgacgggtcgaattctcggaattacagtttgctccccgagaaagcatgaaccacccagatgtcttaactacttgacttcacctcatgttgttatatggagcgcagtcactgcttcttgtgcctttcctggccttttcgaggctcaggaattgatggcaaaggatagaagtggagagattgttccttaccatccaccatttaatttgggtcctgaaaagggttccacttcagtgcgccgttggagggatggtagcttggagatggatttgcctatgatgcaattgaaagagctcttcaatgtcaatcattttatagttagtcaggccaatcctcacattgccccattattgagattcaaagaaattatccgagcttatggaggtaattttgcagcaaagcttgctcatctagcagagatggaggtgaaacatagatgtaatcaagtactggaactcggctttccattaggtggactagccaaactgtttgctcaagactgggagggtgatgtaacagttgttatgcctgcaactcctgcccaatacttaaaaatcatacagaacccttcttatgtagagcttcaaaaggcagctaaccaaggcagaagatgcacctgggagaagctttcggccatgaaagcaaattgtggaattgagcttgctcttgatgagtctgttgcaattctcaatcatatgagacgactcaaaagaaatgctgagagaactgctgctgcttctcatggtcttcccagtacaggcaaatttagtggttcaagaagaattccatcgtggaacatcattgcacgagagaattctacaggctccctagaagaccttcatgctgctgctgctgctacttcctcattgcatcaaggtgttattagtcccagtggagccactggtaaaaattggaagtcccaccgtagctttcatgatgcaagtgacagtgaatctgaaagtgctcatgacttgaattcttggaccagatctggtgggcctttgatgagaactacatcagcagatatgttcattgactttttccagaacatagaagttgataccgaactaaatagaggcaaggtgacaaacactaccactagccctcgtgattttcagcatcacatttcaaggcacacaacacctgatcataataggtggtgctctaagagaacagaaatcgatcagaaagaaaatggcaacaggatgattgccatgaatggatctaacataatggtaactgaaggtgatcttttacagcctgaaaggatccataatgggattgtgtttaatgttgtcaagaaacaagactttttgaccccttcaaataggaataatgattataatagttgttgtaacaatgaagttgctgaatgtgtacaaattgaatttccagggaaggaggtggatgctcttagttcagcttccgaaaatggcgatgatggttccacaatggccaggtccctaactgaaaaaccagattataattcaacaaatcatttcagcaaagattttggtgaagatcaaagcattgctgacaattaa

SEQ ID NO: 92 - DNA, 2523 bp, Glycine max
atggatcatattagtaatgaggctagtattgaccgttttccaattggtccttctgacattcttggtaggacaattgctttcagggttcttttttgcaagtctatctcacattttaggcaccacatatttctagtgttgttagatctcttctatagatttaggggggggttggcatcctttatatcatggttgcatcccaggaaccctcaggggatattggcaatgatgacaattgttgctttcttgttgaaacgatacacaaatgtgaaagcaagggctgaaatggcatataggaggaagttttggagaaacatgatgagaagtgctttgacctatgatgaatgggctcatgcagctaagatgcttgataaagagacaacaaagatgaatgaatcagacctttatgatgtggaattggtgaggaacaagcttcaagagctccgccaccgtcggcaagagggatctcttagagatataatgttttgtatgcgtgccgatcttattagaaatttaggtaatatgtgtaaccctgaactgcacaagggtaggcttcaggtgcctaaattaatcaaggagtacattgatgaagtaacgactcaattgagaatggtctgtgattctgattcagaggagctatcattggaagaaaagcttgctttcatgcatgaaacaaggcatgcatttgggaggacagctttgctgttaagcgggggtgcctctcttggagcttctcatgtgggtgtagttaaaacaatggtagaacataaactcatgcctaggataattgctggttcaagtgtgggatccattatgtgtgctgttgttgccactaggacttggcctgagctccagagcttttttgaggattcatggcactcgttgcaattttttgatcaaatgggtgggatttttgcagttgttaagagagtcacaacattgggtgctgttcatgagatcagacagttgcaaatgatgttgaggcatctaacaagcaatcttacatttcaagaagcttatgacatgacaggtagaattcttgggattacagtttgttccccaaggaagcacgaaccacctagatgtcttaactacttgacttcaccccatgtggttatatggagtgcagtcaccgcttcttgtgccttccctggcctttttgaggctcaagaattgatggcaaaggatagaagtggagagattattccttaccaccctccttttaacttaggtcctgaagagggctccacaccagcacgccgttggagggatggtagcttggagattgatttacctatgatgcagttgaaagaactattcaatgtcaatcattttatagttagtcaggccaaccctcatattgcaccattattgagattgaaagaatttgtgcgcacttatgggggcaacttcgctgccaagcttgctcatcttgtggagatggaggtgaaacataggtgtaatcaaatactggaacttggttttccattaggtggacttgctaagttgtttgctcaagactgggaaggtgatgtgactgttgttattcccgcaactcttgctcagtacaccaaaattatacagaacccttcttatggagagcttcaaaaggcaaccaaccaagggagaagatgtacctgggagaaactttcagccataaaagcaaattgtggcattgagcttgctcttgatgagtgtgtggtgattctcaatcatatgagaagactaaagaggattgctgagagagctgcttctgcttctcaaggtttgtccagcactgtaaggttcagtgcttcaaaaagaattccttcatggaattgcattgcacgagagaattcaactggttcccttgaggaccttactgatgttgctgcctcaaagcatcaaggcattagcagttcctgtggaaccaatggcaaaacttggaagacctaccgtggcatacttgatggaagtgacagtgactctgaaagtgttgatttgcattcttggacaagatctggtgggcctttgatgagaactacttcagcaaatatgtttgtcgattttcttcaaaacttagacgttgataccgatcaaaacaataaaggcttagtgagtcgtgctaaccctaatgattttcagtatcgtagccccaggctcgcaacactggataggaactctgatagcacagaatctgagccaagggaaattggcaacagggttttcaatgtgtccagcatacttgtgaccgaaggtgatcttttgcagcctgaaaggatccataatgggattgtgtttaatgttgtcaagaaagaagacttgtcacccttaagtggtagcagtcatgattttgaaaattacaacattgaagttgctgaatgtgtccaagatgagtgtccagggaaggagatagatgctgctagctcagcttctgaacatggagatgatgaagaatccacagtagctaggtccttaactgaaactcaagactacaattccatggatcatcattcgggtatggatcagagcattgttgacagttag

SEQ ID NO: 93 - DNA, 2535 bp, Glycine max
atgatggatcatattagtaatgaggccagtgttgaccgttttccaattggtccttctggcattcttggtaggacaattgctttcagggttcttttttgcaagtctatctcacattttaggcaccacatatttattgtgttgttagatctcttctataggtttagggggggtttggcatcctttatatcatggttgcatcccaggaaccctcaagggatattggcaatgatgacaattgttgctttcttgttgaaacgatacacaaatgtgaaatcaagggctgaaatggcatataggaggaagttttggagaaacatgatgagaagtgctttgacctatgaggagtgggctcatgcagctaagatgcttgataaagagacaacaaagatgaatgaatcagacctttatgatgtggaattggtgaggaacaagcttcaagagctccgccaccgtcgacaagagggatctctcggagatataatgttttttatgcgtgccgatcttattagaaatttaggtaatatgtgtaaccctgaactacacaagggtaggcttcaggtgcctaaattaatcaaggagtacattgatgaagtaacgactcaattgagaatggtctgtgattctgattcagaggagctatcattggaagaaaagcttgctttcatgcatgaaactaggcatgcatttgggaggactgctttgctgttaagtgggggtgcctctcttggagcttctcatgtgggtgtagttaaaacactggtagaacataaactcatgcctaggataattgctggttcaagtgtgggatccattatgtgtgctgttgttgccactaggacttggcctgagctccagagcttttttgaggattcatggcactcattgcaattttttgatcaaatgggtgggatttttgcagttgttaagagagtcacaacattgggtgctgttcatgagatcagacagttgcaaatgatgttgaggcatctaacaagcaaccttacatttcaagaagcttatgacatgacaggcagaattcttgggattactgtttgttccccaaggaagcatgaaccgcctagatgtcttaactacttgacttcaccccatgtggttatatggagtgcagtaaccgcttcttgtgcctttcctggcctttttgaggctcaagaattgatggcaaaggatagaagtggagagattgttccttaccaccctccttttaacttaggccctgaagagggctccacaccagtgcgccgttggagggatggtagcttggagattgatttacctatgatgcagttgaaagaactattcaatgtcaatcattttatagttagtcaggccaaccctcatattgcaccactattgagattgaaagaatttgtgcggacttatgggggcaactttgctgccaagcttgctcatcttgtggagatggaggtgaaacataggtgtcatcaaatactggaacttggttttccattaggtggacttgctaaattgtttgctcaagactgggaaggtgatgtgactgttgttattcccgcaactcttgctcagtacaccaaaattatacagaacccttcttatggagagcttcaaaaggcagccaaccaagggagaagatgtacctgggagaaactttcagccataaaagcaaattgtggcattgagcttgctcttgatgagtgtgttgtgattctcaatcatatgagaagactaaagagaattgctgagagagctgcttctgcctctcatggtttgtccagcactgtcaggttcagtgcttcaaaaagaattccttcgtggaattgcattgcacgagagaattcgaccggctcccttgaggaccttactgatgttgcctcctcattgcatcaaggcatcggcagttccagcagagccaatggcaaaacttggaagacccaccgtggcatacatgatggaagtgacagtgactctgaaagtgttgatttgcattcttggacaagaactggcgggcctttgatgagaactacttcagcaaatatgttcgttgattttctccaaaacttagaggttgatacggatcctaataaaggcttagtgagtcacactatccataatgattttcagtatcatagccccaggctcacaacactagataggaactctgatagcacagaatctgagccaagggaaactggcaacagggttgtcaatgtgtccagcatacttgtgaccgaaggtgatcttctgcagcctgaaaggatccataatgggattgtgtttaatgttgtcaagaaagaagacttgtcacccttaagtagtagcagtcatggttttgaaaattacaacattgaagttgctgaatgtgtccaagatgagtgtccagggaaggagatagatgctgctagctctgcatctgaacacggagatgatgaagaatccatgccagccaggtccttaactgacatgccagattacaattccattgatcatcattcgggcacagattcgggtatggatcaaagcattgttgacagttag

SEQ ID NO: 94 - DNA, 858 bp, Glycine max
atgggtagaagtcgttgttgttccaaggaagggttgaacaaaggagcatggacagctctggaagatagaattttgacagaatacattaacattcatggcgaaggaaaatggaggcacttgcccaaaagagcaggtcttaagagatgtgggaaaagttgcaggctaagatggttgaattatctgagacctggcattaaaagaggcaacattactaatgatgaagaggagcttataattaggcttcacaatcttcttggaaacaggtggtctttgattgctggacggcttccaggacgaacagacaatgaaatcaagaattattggaacaccaacattgggaggaaacttcaaaatggtggtgcaggaaccactttaaacacacttcaacaagaagatcaaaacgttaaagaacaagagtggcactatgataagggttcgtgtcttgttcagacaaaagcaacaaggtggactaaggtcacagtcaccaatgaaattagtcaacccaatgaagggattacaattaagagcaatgttggtgataacaaaatgaagggttttctagtgtctccatctaaagagagtaacaatgattcagtgttgttggatttcatggcagattttgagatggatggaaacttcttctcggagctccttaagatggatttgcccgaatcatcttgtaaggaaaataagatactcggagataatggtaaccctagcacggttgataaagtctatttatctccaaaatcatcctgtgataccgcccacttcccttggggtgattcattctatgatacaaattttgattttctaccagtgacaatttttatggagtccggatttgattggctctga

SEQ ID NO: 95 - DNA, 822 bp, Glycine max
atgggaagaaaggccaattgtgacaatcaatatgctatgaacagaggtccttggtctgctgaagaagacaaaattcttatgaacgacgttcaagttcatggggaaagaaaatggagagagctttctaaaagagcaggcttgaaaagatgtgggaagagttgtagactccgatggctgaattatctcaagccagatatcaagagaggaaacatttcttcggatgaagaagatctcattatcagactacataagctcttagggaatagatggtctctaattgcgggacgcttaccagggcgaacagacaatgaaattaagaactattggaatacttatttgagaaagaagacagagcacaaggacaagcataacaaaattcttagccataacaacaacattcctatcaaattaagaattgaatctccacagtgctcaaaaaattcctctggtaatgttcttgatcctgcaaaatcccctcacccgatgaggtgtaccaaggttatgatgccagtcaatgattcagtcaacacaatgaatttgattgcaacgagctgggagaatcataacccttctgcatccatgccactagatgaccataatcattgcttaacaggtgtcctccaagaccttgacattagtgacttgttaatgtcgtatgaggatgattgtcgttttaatggatatgcttgtgtcgaaataccccaacatatttttaaagatgagacttggaggcttgatgattcgatgggtgaagaagcatggtatgagaattggaagattgacacctattttccacaagatatggggttttcttcattttaa

SEQ ID NO: 96 - DNA, 786 bp, Glycine max
atgaaaccctctccaaatatgtttccatccatggagaaggaaaatggcagaaggttgcccaaaatgcagctttttggttgtatatatacaataataatgtctgcagggttaaagcgatgtggaaagagttgcagacaaagatggttgaattatctcaagcccggtataaaaagaggccacatctctgtggatgaagaggatatgattataagacttcatcgtcttcttggtaacagatgggcattgattgcaaagaggctacctggacgaacagacaatgaaatcaagaactattggaacacaaatctgtcaaagaagttacaaaaacatccaacatcatcagtttcttcacttcaacacaaacgtcatgaaaaagaaaaaaccatacaaatgcatgtatcacccgaagcaccacggcctaggagagttaatgcagttgaatacagtaaaaatttggagaatggtgggtgtggaaatagacctagtacaactcccagtccctcaaataaagtagagggtagtagtgaagaagcttcttttagtgatttcctcatccacgacattgatcaaaaccaatttttgattgctgatgattcaaactcaaaggtgccacaaatggaagaagatcaccaccacaacaacaaggtggaattaataaccaacaacagtcctagctcttcatcaccttctgatcattgtcaccatctcttgccagagaaatttgacccttttgagacccttttggatgtggagctggcttcctttcttggatttgaaaatgattga

SEQ ID NO: 97 - DNA, 924 bp, Glycine max
atggggagaagcccttgttgttcaaaggagggtttgaatagaggtgcttggacagctcatgaagacaaaatcctgagagaatatattagagtccatggtgaaggaagatggagaaaccttcccaaaagagcaggtttgaaaagatgcgggaaaagttgtagacttcgatggttgaattatctcagaccagatattaagagaggcaatatatccccagatgaagaagagctcatcatcaggctccacaagctcctcggaaacagatggtctttaatagctgggaggcttccaggacgaacagacaacgaaataaagaattattggaacaccaatttagggaaaaaggtgaaagatggtcaccaaacaaccactggaaacaacacacagaatccaatgcccaacccatcaccctccctatctcctcctaaactagattcccatgttgtccgtacaaaggctaccaagtgctccaagttgttattcctaaacccaccccctcacccatcaatgcagaacaagttcaagactgaggcagaagaagaagaagaagaagcaaggcttgtgaatggtgtaattagaaaccaaatggaacacactacatacgacaacgggttcctgtcgtttccggatgaagaaaaagaactctcaacagatttgctcatagatttcaacgtgggggatttctgcttgtctgatcttctcaactcagatttctcaaactcgtacaatttcagctgcaatattaataatgttaacaaccacgagcagttatcgccttgttcggaccaacctgatcctatgttctctgatgaagttctcaaggactggacacacaacaattttgcagacgaagcgaatgcttccaacaatcttcgttctttcatttcgttccttgagtccactgaggaaagattaggagaatga

SEQ ID NO: 98 - protein, 805 aa, Glycine max
MDHISNEASVDPFAIGPSSILGRTIAFRVLFCKSISQLRYHILFVLSNIFKRFRGFWAPIVSWLHPRNQGILAMMTMIAFLLKRYTSVKVRANIAYKRKFWRNMVRAALTYEEWAHAAKMLDRETQKMNESDLYDVELVSNKLEELRHRRQEGSLRDIIFCMRADLIRNLGNMCNPELHKARLQMPRLIKEYIDEVSTQLRMVCNSDSEELALEEKLSFMHETRHAFGRTALLLSGGASLGAFHVGVVKTLVEHKLLPRIIAGSSVGSIMCSIVATRSWPELQSFFEDSLHSLQFFDQMGGIFTVVKRVTTYGAVHEIRQLQMLLRHLTSNLTFQEAYDMTGRILGITVCSPRKHEPPRCLNYLTSPHVVIWSAVTASCAFPGLFEAQELMAKDRSGEIVPYHPPFNLGPEKGSTSVRRWRDGSLENDLPMMQLKELFNVNHFIVSQANPHIAPLLRLKEFVRAYGGNFAAKLAHLAEMEVKHRCNQVLELGFPLGGLAKLFAQDWEGDVTVVMPATLAQYLKIIQNPSYVELQKAANQGRRCTWEKLSAIKANCGIELALDESVAILNHMRRLKRNAERVATASHGLPSTVKFSGSRRIPSWNIIARENSTGSLEDLHADAASSLHQGVISPSGATGKNWKSHRIIHDASDSESETADLNSWTRSGGPLMRTTSADMFIDFVQDLEFHKVNTELNRGKVTHTSPHDFQHHSSRHTTVNRWSESTENDQKENGNRIVMNESNIMVTEGDLLQPERIHNGIVFNVVKKENLTPNRNHDYNSYNNEVAECVQIDFPGKEVDAASSAS

SEQ ID NO: 99 - protein, 758 aa, Glycine max
MDHISNEASVDPFAIGPSSILGRTIAFRVLFCKSISQLRYHILFVLSNIFKRFRGFWAPIVSWLHPRNQGILAMMTMIAFLLKRYTSVKVRANIAYKRKFWRNMVRAALTYEEWAHAAKMLDRETQKMNESDLYDVELVSNKLEELRHRRQEGSLRDIIFCMRADLIRNLGNMCNPELHKARLQMPRLIKEYIDEVSTQLRMVCNSDSEELALEEKLSFMHETRHAFGRTALLLSGGASLGAFHVGVVKTLVEHKLLPRIIAGSSVGSIMCSIVATRSWPELQSFFEDSLHSLQFFDQMGGIFTVVKRVTTYGAVHEIRQLQMLLRHLTSNLTFQEAYDMTGRILGITVCSPRKHEPPRCLNYLTSPHVVIWSAVTASCAFPGLFEAQELMAKDRSGEIVPYHPPFNLGPEKGSTSVRRWRDGSLENDLPMMQLKELFNVNHFIVSQANPHIAPLLRLKEFVRAYGGNFAAKLAHLAEMEVKHRCNQVLELGFPLGGLAKLFAQDWEGDVTVVMPATLAQYLKIIQNPSYVELQKAANQGRRCTWEKLSAIKANCGIELALDESVAILNHMRRLKRNAERVATASHGLPSTVKFSGSRRIPSWNIIARENSTGSLEDLHADAASSLHQGVISPSGATGKNWKSHRIIHDASDSESETADLNSWTRSGGPLMRTTSADMFIDFVQDLEFHKVNTELNRGKVTHTSPHDFQHHSSRHTTVNRWSESTENDQKENGNRIVMNESNIMVTEGKEVDAASSAS

SEQ ID NO: 100 - protein, 799 aa, Glycine max
MDHISNEASVDPFAIGPSSILGRTIAFRVLFCKSISQLRYHILFVLSNIFKRFRGFWAPIVSWLHPRNQGILAMMTMIAFLLKRYTSVKVRANIAYKRKFWRNMVRAALTYEEWAHAAKMLDRETQKMNESDLYDVELVSNKLEELRHRRQEGSLRDIIFCMRADLIRNLGNMCNPELHKARLQMPRLIKEYIDEVSTQLRMVCNSDSEELALEEKLSFMHETRHAFGRTALLLSGGASLGAFHVGVVKTLVEHKLLPRIIAGSSVGSIMCSIVATRSWPELQSFFEDSLHSLQFFDQMGGIFTVVKRVTTYGAVHEIRQLQMLLRHLTSNLTFQEAYDMTGRILGITVCSPRKHEPPRCLNYLTSPHVVIWSAVTASCAFPGLFEAQELMAKDRSGEIVPYHPPFNLGPEKGSTSVRRWRDGSLENDLPMMQLKELFNVNHFIVSQANPHIAPLLRLKEFVRAYGGNFAAKLAHLAEMEVKHRCNQVLELGFPLGGLAKLFAQDWEGDVTVVMPATLAQNPSYVELQKAANQGRRCTWEKLSAIKANCGIELALDESVAILNHMRRLKRNAERVATASHGLPSTVKFSGSRRIPSWNIIARENSTGSLEDLHADAASSLHQGVISPSGATGKNWKSHRIIHDASDSESETADLNSWTRSGGPLMRTTSADMFIDFVQDLEFHKVNTELNRGKVTHTSPHDFQHHSSRHTTVNRWSESTENDQKENGNRIVMNESNIMVTEGDLLQPERIHNGIVFNVVKKENLTPNRNHDYNSYNNEVAECVQIDFPGKEVDAASSAS

SEQ ID NO: 101 - protein, 804 aa, Glycine max
MDHINNEASVDPFAIGPSSILGRTIAFRVLFCKSISQLRHHIHFVLLDILKRFWGFWGPIVSWLHPRNPQGILAMMTIIAVLLKRYSSVKVRAEIAYRQNFWRNMMRAALTYEEWAHAAKMIDRETPKMNESKDLYDVELVSNKLQELRHRRQEGSLRDIIFCMRADLVRNLGNMCNPELHKGRLQMPRLIKEYIDEVSTQLRMVCNSDSEELALEEKLSFMHETRHAFGRTALLLSGGASLGAFHVGVVKTLVEHKLLPRIIAGSSVGSIMCSIVATRSWPELQSFFEDSLHSLQFFDQMGGIFTVVKRVTTYGAVHEIRQLQMLLRNLTSNLTFQEAYDMTGRILGITVCSPRKHEPPRCLNYLTSPHVVIWSAVTASCAFPGLFEAQELMAKDRSGEIVPYHPPFNLGPEKGSTSVRRWRDGSLEMDLPMMQLKELFNVNHFIVSQANPHIAPLLRFKEIIRAYGGNFAAKLAHLAEMEVKHRCNQVLELGFPLGGLAKLFAQDWEGDVTVVMPATPAQYLKIIQNPSYVELQKAANQGRRCTWEKLSAMKANCGIELALDESVAILNHMRRLKRNAERTAAASHGLPSTGKFSGSRRIPSWNIIARENSTGSLEDLHAAAAATSSLHQGVISPSGATGKNWKSHRSFHDASDSESESAHDLNSWTRSGGPLMRTTSADMFIDFFQNIEVDTELNRGKVTNTTTSPRDFQHHISRHTTPDHNRWCSKRTEIDQKENGNRMIAMNGSNIMVTEGKEVDALSSASENGDDGSTMARSLTEKPDYNSTNHFSKDFGEDQSIADN

SEQ ID NO: 102 - protein, 854 aa, Glycine max
MDHINNEASVDPFAIGPSSILGRTIAFRVLFCKSISQLRHHIHFVLLDILKRFWGFWGPIVSWLHPRNPQGILAMMTIIAVLLKRYSSVKVRAEIAYRQNFWRNMMRAALTYEEWAHAAKMIDRETPKMNESKDLYDVELVSNKLQELRHRRQEGSLRDIIFCMRADLVRNLGNMCNPELHKGRLQMPRLIKEYIDEVSTQLRMVCNSDSEELALEEKLSFMHETRHAFGRTALLLSGGASLGAFHVGVVKTLVEHKLLPRIIAGSSVGSIMCSIVATRSWPELQSFFEDSLHSLQFFDQMGGIFTVVKRVTTYGAVHEIRQLQMLLRNLTSNLTFQEAYDMTGRILGITVCSPRKHEPPRCLNYLTSPHVVIWSAVTASCAFPGLFEAQELMAKDRSGEIVPYHPPFNLGPEKGSTSVRRWRDGSLEMDLPMMQLKELFNVNHFIVSQANPHIAPLLRFKEIIRAYGGNFAAKLAHLAEMEVKHRCNQVLELGFPLGGLAKLFAQDWEGDVTVVMPATPAQYLKIIQNPSYVELQKAANQGRRCTWEKLSAMKANCGIELALDESVAILNHMRRLKRNAERTAAASHGLPSTGKFSGSRRIPSWNIIARENSTGSLEDLHAAAAATSSLHQGVISPSGATGKNWKSHRSFHDASDSESESAHDLNSWTRSGGPLMRTTSADMFIDFFQNIEVDTELNRGKVTNTTTSPRDFQHHISRHTTPDHNRWCSKRTEIDQKENGNRMIAMNGSNIMVTEGDLLQPERIHNGIVFNVVKKQDFLTPSNRNNDYNSCCNNEVAECVQIEFPGKEVDALSSASENGDDGSTMARSLTEKPDYNSTNHFSKDFGEDQSIADN

SEQ ID NO: 103 - protein, 840 aa, Glycine max
MDHISNEASIDRFPIGPSDILGRTIAFRVLFCKSISHFRHHIFLVLLDLFYRFRGGLASFISWLHPRNPQGILAMMTIVAFLLKRYTNVKARAEMAYRRKFWRNMMRSALTYDEWAHAAKMLDKETTKMNESDLYDVELVRNKLQELRHRRQEGSLRDIMFCMRADLIRNLGNMCNPELHKGRLQVPKLIKEYIDEVTTQLRMVCDSDSEELSLEEKLAFMHETRHAFGRTALLLSGGASLGASHVGVVKTMVEHKLMPRIIAGSSVGSIMCAVVATRTWPELQSFFEDSWHSLQFFDQMGGIFAVVKRVTTLGAVHEIRQLQMMLRHLTSNLTFQEAYDMTGRILGITVCSPRKHEPPRCLNYLTSPHVVIWSAVTASCAFPGLFEAQELMAKDRSGEIIPYHPPFNLGPEEGSTPARRWRDGSLEIDLPMMQLKELFNVNHFIVSQANPHIAPLLRLKEFVRTYGGNFAAKLAHLVEMEVKHRCNQILELGFPLGGLAKLFAQDWEGDVTVVIPATLAQYTKIIQNPSYGELQKATNQGRRCTWEKLSAIKANCGIELALDECVVILNHMRRLKRIAERAASASQGLSSTVRFSASKRIPSWNCIARENSTGSLEDLTDVAASKHQGISSSCGTNGKTWKTYRGILDGSDSDSESVDLHSWTRSGGPLMRTTSANMFVDFLQNLDVDTDQNNKGLVSRANPNDFQYRSPRLATLDRNSDSTESEPREIGNRVFNVSSILVTEGDLLQPERIHNGIVFNVVKKEDLSPLSGSSHDFENYNIEVAECVQDECPGKEIDAASSASEHGDDEESTVARSLTETQDYNSMDHHSGMDQSIVDS

SEQ ID NO: 104 - protein, 844 aa, Glycine max
MMDHISNEASVDRFPIGPSGILGRTIAFRVLFCKSISHFRHHIFIVLLDLFYRFRGGLASFISWLHPRNPQGILAMMTIVAFLLKRYTNVKSRAEMAYRRKFWRNMMRSALTYEEWAHAAKMLDKETTKMNESDLYDVELVRNKLQELRHRRQEGSLGDIMFFMRADLIRNLGNMCNPELHKGRLQVPKLIKEYIDEVTTQLRMVCDSDSEELSLEEKLAFMHETRHAFGRTALLLSGGASLGASHVGVVKTLVEHKLMPRIIAGSSVGSIMCAVVATRTWPELQSFFEDSWHSLQFFDQMGGIFAVVKRVTTLGAVHEIRQLQMMLRHLTSNLTFQEAYDMTGRILGITVCSPRKHEPPRCLNYLTSPHVVIWSAVTASCAFPGLFEAQELMAKDRSGEIVPYHPPFNLGPEEGSTPVRRWRDGSLEIDLPMMQLKELFNVNHFIVSQANPHIAPLLRLKEFVRTYGGNFAAKLAHLVEMEVKHRCHQILELGFPLGGLAKLFAQDWEGDVTVVIPATLAQYTKIIQNPSYGELQKAANQGRRCTWEKLSAIKANCGIELALDECVVILNHMRRLKRIAERAASASHGLSSTVRFSASKRIPSWNCIARENSTGSLEDLTDVASSLHQGIGSSSRANGKTWKTHRGIHDGSDSDSESVDLHSWTRTGGPLMRTTSANMFVDFLQNLEVDTDPNKGLVSHTIHNDFQYHSPRLTTLDRNSDSTESEPRETGNRVVNVSSILVTEGDLLQPERIHNGIVFNVVKKEDLSPLSSSSHGFENYNIEVAECVQDECPGKEIDAASSASEHGDDEESMPARSLTDMPDYNSIDHHSGTDSGMDQSIVDS

SEQ ID NO: 105 - protein, 285 aa, Glycine max
MGRSRCCSKEGLNKGAWTALEDRILTEYINIHGEGKWRHLPKRAGLKRCGKSCRLRWLNYLRPGIKRGNITNDEEELIIRLHNLLGNRWSLIAGRLPGRTDNEIKNYWNTNIGRKLQNGGAGTTLNTLQQEDQNVKEQEWHYDKGSCLVQTKATRWTKVTVTNEISQPNEGITIKSNVGDNKMKGFLVSPSKESNNDSVLLDFMADFEMDGNFFSELLKMDLPESSCKENKILGDNGNPSTVDKVYLSPKSSCDTAHFPWGDSFYDTNFDFLPVTIFMESGFDWL

SEQ ID NO: 106 - protein, 273 aa, Glycine max
MGRKANCDNQYAMNRGPWSAEEDKILMNDVQVHGERKWRELSKRAGLKRCGKSCRLRWLNYLKPDIKRGNISSDEEDLIIRLHKLLGNRWSLIAGRLPGRTDNEIKNYWNTYLRKKTEHKDKHNKILSHNNNIPIKLRIESPQCSKNSSGNVLDPAKSPHPMRCTKVMMPVNDSVNTMNLIATSWENHNPSASMPLDDHNHCLTGVLQDLDISDLLMSYEDDCRFNGYACVEIPQHIFKDETWRLDDSMGEEAWYENWKIDTYFPQDMGFSSF

SEQ ID NO: 107 - protein, 261 aa, Glycine max
MKPSPNMFPSMEKENGRRLPKMQLFGCIYTIIMSAGLKRCGKSCRQRWLNYLKPGIKRGHISVDEEDMIIRLHRLLGNRWALIAKRLPGRTDNEIKNYWNTNLSKKLQKHPTSSVSSLQHKRHEKEKTIQMHVSPEAPRPRRVNAVEYSKNLENGGCGNRPSTTPSPSNKVEGSSEEASFSDFLIHDIDQNQFLIADDSNSKVPQMEEDHHHNNKVELITNNSPSSSSPSDHCHHLLPEKFDPFETLLDVELASFLGFEND

SEQ ID NO: 108 - protein, 307 aa, Glycine max
MGRSPCCSKEGLNRGAWTAHEDKILREYIRVHGEGRWRNLPKRAGLKRCGKSCRLRWLNYLRPDIKRGNISPDEEELIIRLHKLLGNRWSLIAGRLPGRTDNEIKNYWNTNLGKKVKDGHQTTTGNNTQNPMPNPSPSLSPPKLDSHVVRTKATKCSKLLFLNPPPHPSMQNKFKTEAEEEEEEARLVNGVIRNQMEHTTYDNGFLSFPDEEKELSTDLLIDFNVGDFCLSDLLNSDFSNSYNFSCNINNVNNHEQLSPCSDQPDPMFSDEVLKDWTHNNFADEANASNNLRSFISFLESTEERLGE

SEQ ID NO: 109 - protein, 17 aa, Artificial Sequence
  Synthetic construct nuclear localization sequence
MAPKKKRKVGIHGVPAA

SEQ ID NO: 110 - DNA, 12 bp, Camelina sativa
aaggatacttgg

SEQ ID NO: 111 - DNA, 13 bp, Camelina sativa
aaggattacttgg

SEQ ID NO: 112 - DNA, 20 bp, Artificial Sequence
  Synthetic construct CsC1-69 guide target sequence
ggttgttgtcgaagttttag

SEQ ID NO: 113 - DNA, 20 bp, Artificial Sequence
  Synthetic construct CsC2-33 guide target sequence
gctcattcccaagtcctctg

SEQ ID NO: 114 - DNA, 20 bp, Artificial Sequence
  Synthetic construct CsC3-52 guide target sequence
gatcccttgctacatatagg

SEQ ID NO: 115 - DNA, 20 bp, Artificial Sequence
  Synthetic construct C1-29A guide target sequence
gtacttcttgtgtaccacgg

SEQ ID NO: 116 - DNA, 20 bp, Artificial Sequence
  Synthetic construct C1-29B guide target sequence
gtacttcttgcgttccacgg

(19) United States
(12) Patent Application Publication
(10) Pub. No.: US 2022/0403403 A1
(43) Pub. Date: Dec. 22, 2022

(54) GENETICALLY MODIFIED PLANTS THAT EXHIBIT AN INCREASE IN SEED YIELD COMPRISING A FIRST HOMEOLOG OF SUGAR-DEPENDENT1 ( SDP1) HOMOZYGOUS FOR A WILD-TYPE ALLELE AND A SECOND HOMEOLOG OF SDP1 HOMOZYGOUS FOR A MUTANT ALLELE

(71) Applicant: YIELD10 BIOSCIENCE, INC., Woburn, MA (US)

(72) Inventors: Meghna MALIK, Saskatoon (US); Jihong TANG, West Roxbury, MA (US); Yuanyuan JI, (US); Kristi D. SNELL, Belmont, MA (US)

(21) Appl. No.: 17/597,707

(22) PCT Filed: Jul. 22, 2020
(86) PCT No.: PCT/US2020/043063

§ 371 Date: Jan. 19, 2022

Related U.S. Application Data
(60) Provisional application No. 62/877,591, filed on Jul. 23, 2019.

(51) Int. Cl. C12N 15/82

(52) U.S. Cl. CPC C12N 15/8261; C12N 15/8247; C12N 15/8213

(57) ABSTRACT

A genetically modified plant that exhibits an increase in seed yield relative to a progenitor plant is disclosed. The genetically modified plant includes (a) a first homeolog of the SUGAR-DEPENDENT1 (SDP1) gene being homozygous for a wild-type allele; and (b) a second homeolog of the SDP1 gene being homozygous for a mutant allele. The wild-type allele encodes an active SDP1 triacylglycerol lipase and is identical to an allele of the first homeolog from the progenitor plant. The mutant allele does not encode an active SDP1 triacylglycerol lipase and includes one or more additions, deletions, or substitutions of one or more nucleotides relative to an allele of the second homeolog from the progenitor plant. The genetically modified plant expresses about 20% to 80% of SDP1 triacylglycerol lipase activity in seeds relative to the progenitor. The increase in seed yield is at least 10%.

### STATEMENT OF GOVERNMENT SPONSORED RESEARCH

This invention was made with government support under Contract No. DE-EE0007003 awarded by the United States Department of Energy. The government has certain rights in the invention.

### FIELD OF THE INVENTION

The present invention relates generally to genetically modified plants that exhibit an increase in seed yield relative to a progenitor plant from which the genetically modified plants were derived, and more particularly to such genetically modified plants comprising: (a) a first homeolog of the SUGAR-DEPENDENT1 (SDP1) gene, occurring in its natural position within the genome of the genetically modified plant and being homozygous for a wild-type allele; and (b) a second homeolog of the SDP1 gene, occurring in its natural position within the genome of the genetically modified plant and being homozygous for a mutant allele.

### BACKGROUND OF THE INVENTION

Vegetable oils are an important renewable source of hydrocarbons for food, energy, and industrial feedstocks. As demand for this commodity increases, discovering ways to increase vegetable oil production in an oilseed crop will be an agronomic priority. With the increasing global population and the added infrastructure impact on arable land available for crop production it will be critical to increase the amount of harvestable vegetable oil from each acre of land. Vegetable oil per acre of land is determined by the yield of oilseed per acre multiplied by the oil content (usually stated as a percentage of dry seed weight). Increasing vegetable oil per acre can be accomplished in a number of ways: (1) developing new oilseed varieties which produce higher seed yield without reducing the seed oil content; (2) developing new oilseed varieties that have higher seed oil content without reducing seed yield; or (3) developing new oilseed varieties that have higher seed oil content and higher seed yield. The net impact of any of these three solutions will be to increase vegetable oil production per harvestable acre of land.

The production of oil in plants is a dynamic process involving multiple metabolic pathways including the fatty acid biosynthesis pathway, triacylglycerol (also termed “TAG”) biosynthesis, and TAG degradation, and complex gene regulation systems. During the production of oil in an oilseed, the rate of fatty acid and TAG biosynthesis is high and the rate of TAG degradation is low, resulting in a net accumulation of oil. TAG degradation is an essential process for seed germination.

Genes involved in the production of oil in plants include, among others, the following: (i) SUGAR DEPENDENT1 (also termed “SDP1” or “sdp1”) and SUGAR-DEPENDENT1-LIKE (also termed “SDP 1-L,” “sdp1-L,” “SDP1-Like” or “sdp1-like”) genes, which encode oil body-associated triacylglycerol lipases (Eastmond, 2006, Plant Cell, 18, 665); (ii) TRANSPARENT TESTA2 (also termed “TT2” or “tt2”) genes, which encode a transcription factor that coordinates gene expression for fatty acid biosynthesis in the embryo and proanthocyanidins in the seed coat (Chen et al., Plant Physiology, 2012, 160, 1023); and (iii) genes encoding biotin/lipoyl attachment domain-containing (also termed “BADC” or “badc”) proteins, which are negative regulators of the acetyl-CoA carboxylase enzyme (PCT/US2016/041386 to the University of Missouri (published as WO2017/039834)).

Regarding SDP1 and SDP1-L genes, triacylglycerol content in oil seeds is highest during the late maturation phase, but in many species declines during the following desiccation phase, which is when the seeds typically dry down before being harvested. This loss can account for about 10% of the maximum oil content in Brassica napus seeds grown in the greenhouse or in the field (Chia et al., 2005, Journal of Experimental Botany, 56, 1285; Kelly et al., 2013, Plant Biotechnology Journal, 11, 355). Oil catabolism (degradation) is initiated by triacylglycerol lipases that hydrolyze fatty acids off the glycerol backbone for subsequent conversion into sugars or amino acids via β-oxidation, glyoxylate cycle, and gluconeogenesis.

Two oil body-associated triacylglycerol lipases, SDP1 and SDP1-L, have been identified in Arabidopsis thaliana. Both enzymes together contribute over 95% of triacylglycerol lipase activity during seed germination (Eastmond, 2006, Plant Cell, 18, 665). Knockout mutants of SDP1 in Arabidopsis thaliana (sdp1-5) were delayed in germination due to reduced rates of oil degradation, but had no phenotype once photosynthesis contributed to carbon supply, and SDP1-like null mutants had no growth and developmental phenotype (Kelly et al., 2011, Plant Physiology, 157, 866). Both genes are also highly expressed during seed maturation and desiccation in Arabidopsis thaliana, suggesting their involvement in oil loss during desiccation. Desiccated seeds of the Arabidopsis SDP1 null mutant sdp1-5 were larger and had 11.5% higher seed weight per seed as compared to wild-type seeds. No changes in seed yield per plant were reported (Kelly et al., 2011). The dry seeds contained 10% more total lipids, with an increased proportion of TAG and corresponding decrease in free fatty acids (Kim et al., 2014, Biotechnology for Biofuels, 7, 36). Similar results were obtained by antisense repression of SDP1, driven by a seed-specific promoter in Arabidopsis thaliana, which increased the TAG content in desiccated seeds by about 10% without affecting germination or growth rate of seedlings (van Erp et al., 2014, Plant Physiology, 165, 30). In that case, it was noted that although evidence was provided that an increase in seed oil content can translate into greater oil yield, the relationship is very likely to be less than proportionate, and that no significant increase in seed yield (P>0.05) could be detected in any of the engineered lines, but a significant (P<0.05) reduction in seed number was apparent in most of the engineered lines (van Erp et al., 2014). A similar antisense-RNA approach using a conserved region to repress three putative alleles of SDP1 in Brassica napus driven by a seed maturation specific promoter led to increases of seed oil content between 3% and 8% on a per seed and per plant basis with slight reductions in seed protein content 4%) (Kelly et al., 2013). The most dramatic increases in seed oil content were achieved using antisense repression of the SDP1-homolog in Jatropha curcas driven by its endogenous promoter (Kim et al., 2014). The JcSDP1 protein levels were reduced by only 7%, which resulted in an increase in total lipid content in the transgenic endosperm of up to 30% compared to control lines without affecting germination, growth rates, or any other phenotypic traits (Kim et al., 2014).

Regarding TT2 genes, during seed development and maturation incoming carbohydrate supply is directed into different pathways by transcriptional master regulators. One of these master regulators is TT2, a transcription factor that coordinates the gene expression of enzymes for the proanthocyanidins (PAs) in the seed coat and fatty acid biosynthesis in the embryo (Chen et al., 2012). While TT2 activates the biosynthesis pathway of PAs in the seed coat, it represses the expression of the fatty acid biosynthesis pathway enzymes in the embryo by inhibiting the activity of the transcription factor FUSCA3 (Chen et al., 2012). Consequently, as shown in TT2 Arabidopsis null mutants, expression of FUSCA3 is increased and leads to an increase in fatty acid biosynthesis in the seed embryo (Wang et al., 2014, The Plant Journal, 77, 757). Null mutants of TT2 lack the dark brown color of the condensed tannins (oxidized PAs) in the maternal seed coat and are therefore easily identified in T₂ seeds (Debeaujon et al., 2003, The Plant Cell Online, 15, 2514). Analysis of seed composition showed that TT2 knockout lines contain up to 79% more fatty acids (based on seed dry weight) compared to wild-type while their protein content was reduced by more than 50%. Most of the increased fatty acids were found to be long-chain (C20) and very-long chain fatty acids (C22; C24) (Chen et al., 2012; Wang et al., 2014). Germination and development of TT2 knockout seeds and plants were not affected by the mutation, but germination rate was slightly delayed under salt stress conditions.

Regarding genes encoding BADC proteins, the BADC proteins are negative regulators of the acetyl-coA carboxylase enzyme which catalyzes the first committed step in fatty acid biosynthesis. Fatty acids are the key precursors for oil biosynthesis in oilseeds. It has been shown that reducing the expression of BADC in Arabidopsis results in an increase in seed oil content. It has also been shown that gene knockouts of BADC in Arabidopsis result in increased fatty acid production and oil content in seeds but with lower seed yield (PCT/US2016/041386 to the University of Missouri; Keereetaweep et al., 2018, Plant Physiology, 177, 208).

There is a need to develop plants in which the TAG production rates are increased and TAG degradation rates during seed production are decreased without impairing overall seed yield and preferably increasing overall seed yield.

### BRIEF SUMMARY OF THE INVENTION

A genetically modified plant that exhibits an increase in seed yield relative to a progenitor plant from which the genetically modified plant was derived is provided. The genetically modified plant comprises (a) a first homeolog of the SUGAR-DEPENDENT1 (SDP1) gene, occurring in its natural position within the genome of the genetically modified plant and being homozygous for a wild-type allele; and (b) a second homeolog of the SDP1 gene, occurring in its natural position within the genome of the genetically modified plant and being homozygous for a mutant allele. The wild-type allele encodes an active SDP1 triacylglycerol lipase and is identical to an allele of the first homeolog of the SDP1 gene from the progenitor plant. The mutant allele does not encode an active SDP1 triacylglycerol lipase and includes one or more additions, deletions, or substitutions of one or more nucleotides relative to an allele of the second homeolog of the SDP1 gene from the progenitor plant. The genetically modified plant expresses about 20% to 80% of SDP1 triacylglycerol lipase activity in seeds relative to the progenitor. The increase in seed yield is at least 10%.

In some embodiments, the genetically modified plant comprises the first homeolog and the second homeolog based on one or more of polyploidy, alloploidy, autoploidy, diploidization following polyploidy, diploidization following alloploidy, or diploidization following autoploidy. In some embodiments, the genetically modified plant is allotetetraploid, allohexaploid, or allooctoploid.

In some embodiments, the genetically modified plant is homozygous for the wild-type allele based on including two identical copies of a wild-type allele. In some embodiments, the genetically modified plant is homozygous for the wild-type allele based on including a first wild-type allele and a second wild-type allele that are not identical to each other.

In some embodiments, the genetically modified plant is homozygous for the mutant allele based on including two copies of the mutant allele that are identical. In some embodiments, the genetically modified plant is homozygous for the mutant allele based on including a first mutant allele and a second mutant allele that are not identical to each other.

In some embodiments, the active SDP1 triacylglycerol lipase has a sequence that is at least 70% identical to one or more SEQ ID NO: 30, SEQ ID NO: 31, or SEQ ID NO: 32. In some embodiments, the active SDP1 triacylglycerol lipase has a sequence that comprises SEQ ID NO: 30, SEQ ID NO: 31, or SEQ ID NO: 32.

In some embodiments, the one or more additions, deletions, or substitutions of one or more nucleotides comprise one or more of a frameshift mutation, an active site mutation, a nonconservative substitution mutation, or an open-reading-frame deletion mutation in the mutant allele relative to the allele of the second homeolog of the SDP1 gene from the progenitor plant.

In some embodiments, the genetically modified plant expresses about 30% to 70% of SDP1 triacylglycerol lipase activity in seeds relative to the progenitor.

In some embodiments, the increase in seed yield is at least 20%, 25%, 30%, 35%, 40%, 45%, 50%, or more.

In some embodiments the genetically modified plant further comprises a third homeolog of the SDP1 gene occurring in its natural position within the genome of the genetically modified plant. In some of these embodiments, the third homeolog is homozygous for a wild-type allele. In some of these embodiments, the third homeolog is homozygous for a mutant allele. In some of these embodiments, the third homeolog is heterozygous for a wild-type allele and a mutant allele.

In some embodiments, the genetically modified plant further comprises: (a) a first homeolog of the SUGAR-DEPENDENT1-LIKE (SDP1-L) gene, occurring in its natural position within the genome of the genetically modified plant and being homozygous for a wild-type allele; and (b) a second homeolog of the SDP1-L gene, occurring in its natural position within the genome of the genetically modified plant and being homozygous for a mutant allele. In these embodiments, the wild-type allele encodes an active SDP1-L triacylglycerol lipase and is identical to an allele of the first homeolog of the SDP1-L gene from the progenitor plant. Also in these embodiments, the mutant allele does not encode an active SDP1-L triacylglycerol lipase and includes one or more additions, deletions, or substitutions of one or more nucleotides relative to an allele of the second homeolog of the SDP1-L gene from the progenitor plant.

In some embodiments, the genetically modified plant further comprises: (a) a first homeolog of the TRANSPARENT TESTA2 (TT2) gene, occurring in its natural position within the genome of the genetically modified plant and being homozygous for a wild-type allele; and (b) a second homeolog of the TT2 gene, occurring in its natural position within the genome of the genetically modified plant and being homozygous for a mutant allele. In these embodiments, the wild-type allele encodes an active TT2 transcription factor and is identical to an allele of the first homeolog of the TT2 gene from the progenitor plant. Also in these embodiments, the mutant allele does not encode an active TT2 transcription factor and includes one or more additions, deletions, or substitutions of one or more nucleotides relative to an allele of the second homeolog of the TT2 gene from the progenitor plant.

In some embodiments, the genetically modified plant is one or more of a Brassica species, Brassica napus, Brassica rapa, Brassica carinata, Brassica juncea, Camelina sativa, a Crambe species, a Jatropha species, pennycress, Ricinus communis, a Calendula species, a Cuphea species, Arabidopsis thaliana, maize, soybean, a Gossypium species, sunflower, palm, coconut, safflower, peanut, Sinapis alba, sugarcane, flax, or tobacco. In some embodiments, the genetically modified plant is Brassica napus, Brassica rapa, Brassica carinata, Brassica juncea, Camelina sativa, or soybean.

In some embodiments, the genetically modified plant is Camelina sativa. In some of these embodiments, the natural position of the second homeolog of the SDP1 gene is on chromosome 13 of Camelina sativa. Also in some of these embodiments, the allele of the second homeolog of the SDP1 gene from the progenitor plant encodes a protein that has a sequence comprising SEQ ID NO: 31. Also in some of these embodiments, the allele of the second homeolog of the SDP1 gene from the progenitor plant comprises SEQ ID NO: 2. Also in some of these embodiments, the genetically modified plant further comprises a third homeolog of the SDP1 gene occurring in its natural position within the genome of the genetically modified plant, wherein the third homeolog is homozygous for a wild-type allele.

Example embodiments include the following:

Embodiment 1: A genetically modified plant that exhibits an increase in seed yield relative to a progenitor plant from which the genetically modified plant was derived, the genetically modified plant comprising:
(a) a first homeolog of the SUGAR-DEPENDENT1 (SDP1) gene, occurring in its natural position within the genome of the genetically modified plant and being homozygous for a wild-type allele; and
(b) a second homeolog of the SDP1 gene, occurring in its natural position within the genome of the genetically modified plant and being homozygous for a mutant allele, wherein:

(i) the wild-type allele encodes an active SDP1 triacylglycerol lipase and is identical to an allele of the first homeolog of the SDP1 gene from the progenitor plant;
(ii) the mutant allele does not encode an active SDP1 triacylglycerol lipase and includes one or more additions, deletions, or substitutions of one or more nucleotides relative to an allele of the second homeolog of the SDP1 gene from the progenitor plant;
(iii) the genetically modified plant expresses about 20% to 80% of SDP1 triacylglycerol lipase activity in seeds relative to the progenitor; and
(iv) the increase in seed yield is at least 10%.

Embodiment 2: The genetically modified plant of embodiment 1, wherein the genetically modified plant comprises the first homeolog and the second homeolog based on one or more of polyploidy, alloploidy, autoploidy, diploidization following polyploidy, diploidization following alloploidy, or diploidization following autoploidy.

Embodiment 3: The genetically modified plant of embodiment 1, wherein the genetically modified plant is allotetetraploid, allohexaploid, or allooctoploid.

Embodiment 4: The genetically modified plant of any one of embodiments 1-3, wherein the genetically modified plant is homozygous for the wild-type allele based on including two identical copies of a wild-type allele.

Embodiment 5: The genetically modified plant of any one of embodiments 1-3, wherein the genetically modified plant is homozygous for the wild-type allele based on including a first wild-type allele and a second wild-type allele that are not identical to each other.

Embodiment 6: The genetically modified plant of any one of embodiments 1-5, wherein the genetically modified plant is homozygous for the mutant allele based on including two copies of the mutant allele that are identical.

Embodiment 7: The genetically modified plant of any one of embodiments 1-5, wherein the genetically modified plant is homozygous for the mutant allele based on including a first mutant allele and a second mutant allele that are not identical to each other.

Embodiment 8: The genetically modified plant of any one of embodiments 1-7, wherein the active SDP1 triacylglycerol lipase has a sequence that is at least 70% identical to one or more SEQ ID NO: 30, SEQ ID NO: 31, or SEQ ID NO: 32.

Embodiment 9: The genetically modified plant of embodiment 8, wherein the active SDP1 triacylglycerol lipase has a sequence that comprises SEQ ID NO: 30, SEQ ID NO: 31, or SEQ ID NO: 32.

Embodiment 10: The genetically modified plant of any one of embodiments 1-9, wherein the one or more additions, deletions, or substitutions of one or more nucleotides comprise one or more of a frameshift mutation, an active site mutation, a nonconservative substitution mutation, or an open-reading-frame deletion mutation in the mutant allele relative to the allele of the second homeolog of the SDP1 gene from the progenitor plant.

Embodiment 11: The genetically modified plant of any one of embodiments 1-10, wherein the genetically modified plant expresses about 30% to 70% of SDP1 triacylglycerol lipase activity in seeds relative to the progenitor.

Embodiment 12: The genetically modified plant of any one of embodiments 1-11, wherein the increase in seed yield is at least 20%.

Embodiment 13: The genetically modified plant of any one of embodiments 1-12, further comprising a third homeolog of the SDP1 gene occurring in its natural position within the genome of the genetically modified plant.

Embodiment 14: The genetically modified plant of embodiment 13, wherein the third homeolog is homozygous for a wild-type allele.

Embodiment 15: The genetically modified plant of embodiment 13, wherein the third homeolog is homozygous for a mutant allele.

Embodiment 16: The genetically modified plant of embodiment 13, wherein the third homeolog is heterozygous for a wild-type allele and a mutant allele.

Embodiment 17: The genetically modified plant of any one of embodiments 1-16, further comprising:
(a) a first homeolog of the SUGAR-DEPENDENT1-LIKE (SDP1-L) gene, occurring in its natural position within the genome of the genetically modified plant and being homozygous for a wild-type allele; and
(b) a second homeolog of the SDP1-L gene, occurring in its natural position within the genome of the genetically modified plant and being homozygous for a mutant allele, wherein:
(i) the wild-type allele encodes an active SDP1-L triacylglycerol lipase and is identical to an allele of the first homeolog of the SDP1-L gene from the progenitor plant; and
(ii) the mutant allele does not encode an active SDP1-L triacylglycerol lipase and includes one or more additions, deletions, or substitutions of one or more nucleotides relative to an allele of the second homeolog of the SDP1-L gene from the progenitor plant.

Embodiment 18: The genetically modified plant of any one of embodiments 1-17, further comprising:
(a) a first homeolog of the TRANSPARENT TESTA2 (TT2) gene, occurring in its natural position within the genome of the genetically modified plant and being homozygous for a wild-type allele; and
(b) a second homeolog of the TT2 gene, occurring in its natural position within the genome of the genetically modified plant and being homozygous for a mutant allele, wherein:
(i) the wild-type allele encodes an active TT2 transcription factor and is identical to an allele of the first homeolog of the TT2 gene from the progenitor plant; and
(ii) the mutant allele does not encode an active TT2 transcription factor and includes one or more additions, deletions, or substitutions of one or more nucleotides relative to an allele of the second homeolog of the TT2 gene from the progenitor plant.

Embodiment 19: The genetically modified plant of any one of embodiments 1-18, wherein the genetically modified plant is one or more of a Brassica species, Brassica napus, Brassica rapa, Brassica carinata, Brassica juncea, Camelina sativa, a Crambe species, a Jatropha species, pennycress, Ricinus communis, a Calendula species, a Cuphea species, Arabidopsis thaliana, maize, soybean, a Gossypium species, sunflower, palm, coconut, safflower, peanut, Sinapis alba, sugarcane, flax, or tobacco.

Embodiment 20: The genetically modified plant of embodiment 19, wherein the genetically modified plant is Brassica napus, Brassica rapa, Brassica carinata, Brassica juncea, Camelina sativa, or soybean.

Embodiment 21: The genetically modified plant of embodiment 1, wherein the genetically modified plant is Camelina sativa.

Embodiment 22: The genetically modified plant of embodiment 21, wherein the natural position of the second homeolog of the SDP1 gene is on chromosome 13 of Camelina sativa.

Embodiment 23: The genetically modified plant of embodiment 21 or 22, wherein the allele of the second homeolog of the SDP1 gene from the progenitor plant encodes a protein that has a sequence comprising SEQ ID NO: 31.

Embodiment 24: The genetically modified plant of any one of embodiments 21-23, wherein the allele of the second homeolog of the SDP1 gene from the progenitor plant comprises SEQ ID NO: 2.

Embodiment 25: The genetically modified plant of any one of embodiments 21-24, further comprising a third homeolog of the SDP1 gene occurring in its natural position within the genome of the genetically modified plant, wherein the third homeolog is homozygous for a wild-type allele.

### BRIEF DESCRIPTION OF THE DRAWINGS

FIG. 1 illustrates targets for CRISPR/Cas9 gene edits to significantly increase oil content and/or seed yield and their function in specific parts of plant metabolism. Stacking edits of sdp1, sdp1-like, and tt2 that have a role in carbon flow to fatty acid biosynthesis, seed coat pigmentation, and lipase activity during various stages of seed development, is expected to increase total oil content in seeds. Adding a badc edit to the sdp1, sdp1-like, and tt2 edited lines will increase carbon flow into fatty acid biosynthetic pathways.

FIG. 2A-D shows a multiple sequence alignment of the Arabidopsis thaliana SDP1 and SDP1-like proteins with seven Camelina orthologs according to CLUSTAL O (1.2.4). Sequence descriptions and SEQ ID numbers are shown in TABLE 1 and TABLE 2. The sequences are as follows: ARABIDOPSIS_SDP1 (SEQ ID NO: 28); Camelina SDP1_CH_8 (SEQ ID NO: 30); Camelina SDP1_CH_13 (SEQ ID NO: 31); Camelina SDP1_CH_20 (SEQ ID NO: 32); ARABIDOPSIS_SDP1-LIKE (SEQ ID NO: 29); Camelina SDP1-like_CH_9 (SEQ ID NO: 37); Camelina SDP1-LIKE_CH_4 (SEQ ID NO: 33); Camelina SDP1-LIKE_CH_6_ISO_X1 (SEQ ID NO: 34); and Camelina SDP1-LIKE_CH_6_ISO_X2 (SEQ ID NO: 36).

FIG. 3 illustrates the genetic elements transformed into plants to achieve Cas9 mediated genome editing. A. Separate cassettes for expression of a DNA molecule encoding a single guide RNA (sgRNA) and a gene encoding the Cas9 enzyme. The expression cassette for the sgRNA is composed of DNA encoding a guide target sequence, targeted to the gene of interest in the Camelina genome, fused to DNA encoding a guide RNA scaffold. The DNA encoding the guide portion of the sgRNA is often identical to the “guide target sequence” of the genomic DNA to be cut, however several mismatches, depending on their position, can be tolerated and still promote double stranded DNA cleavage. B. An sgRNA and Cas9 enzyme are produced. C. Pairing of the sgRNA to genomic DNA at the target site, which lies adjacent to a protospacer adjacent motif (PAM) site, an additional requirement for target recognition. A double stranded DNA break will occur at a position within the Guide target site.

FIG. 4 illustrates plasmid maps of binary vectors for Cas9 mediated genome editing of (A) sdp1 gene in Camelina sativa and (B) sdp1-like gene in Camelina sativa. (A) Binary construct pMBXS1107 (SEQ ID NO: 5) for Cas9 mediated genome editing of the coding sequence of the sdp1 genes using guide target sequence SDP1 #71 (TABLE 3, SEQ ID NO: 4). Important genetic elements within the vector are as follows: U6-26p, a DNA fragment encoding the polymerase III promoter from the Arabidopsis U6-26 small nuclear RNA gene; Guide SDP1 #71 (SEQ ID NO: 4), DNA encoding a 20 bp guide target sequence; gRNA Sc, DNA fragment encoding a Guide RNA scaffold encoding a crRNA-tracrRNA hybrid engineered from the Streptococcus pyogenes CRISPR locus (the DNA encoding the guide target sequence and gRNASc, when expressed together, form a functional sgRNA sequence); U6-26t, a DNA fragment encoding the terminator from the Arabidopsis U6-26 snRNA gene; 35S:C4PPDK promoter (Chiu et al., 1996, Curr. Biol., 6, 325); 2X Flag, a fragment encoding a FLAG polypeptide protein tag (Li et al., 2013, Nature Biotechnology, 31, 688) created by artificial design (Hopp et al., 1988, Bio/Technology, 6, 1204); NLS-5′, a nuclear localization sequence encoding the peptide MAPKKKRKVGIHGVPAA (SEQ ID NO: 109) (WO 2016114972) attached to the 5′ end of Cas9; pcoCas9-5′, DNA fragment encoding the 5′ part of a Cas9 (CRISPR associated protein 9) from Streptococcus pyogenes codon-optimized for expression in plants (pcoCas9, Li et al., 2013, Nature Biotechnology, 31, 688); IV2, a DNA sequence encoding the second intron (IV2) of the nuclear photosynthetic gene ST-LS1 from Solanum tuberosum (Vancanneyt et al., 1990, Molecular and General Genetics, 220, 245); pcoCas9-3′, DNA fragment encoding the 3′ part of Cas9 (the 5′ fragment and the 3′ fragment of pcoCas9 together form the complete Cas9 protein coding sequence); NLS-3′, DNA fragment encoding the nuclear localization sequence of nucleoplasmin, a protein involved in chromatin assembly and histone storage in the Xenopus oocyte and egg (Dingwall et al., 1988, Journal of Cell Biology, 1988, 107, 841), attached to the 3′ end of Cas9; nos, a termination sequence. An expression cassette for the DsRed protein driven by the 2X CaMV 35S promoter provides a visual selection of transgenic seeds. (B) Binary construct pMBXS1126 (SEQ ID NO: 6) for Cas9 mediated genome editing of the coding sequence of the Camelina sativa sdp1-like genes using guide target sequence SDP1-like #4 (TABLE 8, SEQ ID NO: 10). Important genetic elements within the vector are as follows and are described in more detail above: Promoter U6-26p; Guide SDP1-like #4 (SEQ ID NO: 10), DNA encoding a 20 bp guide target sequence to the sdp1-like genes; gRNA Sc; U6-26t; 35S:C4PPDK promoter; 2XFlag, NLS-5′ nuclear localization sequence; pcoCas9-5′ encoding the 5′ part of the Cas9 protein; the IV2 intron sequence; pcoCas9-3′ encoding the 3′ part of Cas9; NLS-3′ nuclear localization sequence; nos termination sequence. An expression cassette for the DsRed protein driven by the 2X CaMV 35S promoter provides a visual selection of transgenic seeds.

FIG. 5 shows the expression profiles of the three different homeologs of SDP1 on Chromosomes 8, 13, and 20 according to the Camelina eFP Browser (website: //bar.utoronto.ca/efp_camelina/cgi-bin/efpWeb.cgi). The expression signal is in units of FPKM, fragments per kilobase of transcript per million mapped reads.

FIG. 6 illustrates the binary construct pMBXS1140 (SEQ ID NO: 16) designed for Cas9 mediated genome editing of the coding sequences of the sdp1, sdp1-like, and tt2 genes in Camelina sativa WT43. Important genetic elements within the vector are as follows and are described in more detail above regarding FIG. 4: Promoter U6-26p; Guide TT2#106/107 (SEQ ID NO: 15), DNA encoding a 20 bp guide target sequence to the tt2 genes; gRNA Sc; U6-26t; Promoter U6-26p; Guide SDP1-like #4 (SEQ ID NO: 10), DNA encoding a 20 bp guide target sequence to the sdp1-like genes; gRNA Sc; U6-26t; Promoter U6-26p; Guide SDP1 #77 (SEQ ID NO: 14), DNA encoding a 20 bp guide target sequence to the sdp1 genes; gRNA Sc; U6-26t; 35S:C4PPDK promoter; 2XFlag, NLS-5′ nuclear localization sequence; pcoCas9-5′ encoding the 5′ part of the Cas9 protein; the IV2 intron sequence; pcoCas9-3′ encoding the 3′ part of Cas9; NLS-3′ nuclear localization sequence; nos termination sequence. An expression cassette for the DsRed protein driven by the 2X CaMV 35S promoter provides a visual selection of transgenic seeds.

FIG. 7 illustrates the seed coat phenotype of T3 seeds harvested from T2 multiplex edited lines targeting the sdp1, sdp1-like, and tt2 genes. A loss of pigmentation in the seed coat is observed in lines with 100% editing within the tt2 gene (lines 17-1013, 17-1011 and 17-1014; TABLE 13) compared to lines with partial tt2 editing (lines 17-1012 and 17-1042; TABLE 13) and WT43.

FIG. 8 illustrates the development of stable, fertile homozygous lines with INDELS in the sdp1, sdp1-like, and tt2 gene targets. INDELS is an abbreviation for insertions or deletions.

FIG. 9 illustrates plasmid maps of binary constructs (A) pMBXO58 (SEQ ID NO: 18) expressing the CCP1 gene from the 35S constitutive promoter, and (B) pMBXO84 (SEQ ID NO: 19) expressing the CCP1 gene from the seed specific promoter from the soya bean oleosin isoform A gene. A. Plasmid pMBXO58 contains a CaMV35S constitutive promoter operably linked to the CCP1 gene from Chlamydomonas reinhardtii fused to a C-terminal myc tag operably linked to an OCS3 termination sequence. An expression cassette for the bar gene, driven by the mannopine synthase promoter, imparts transgenic plants resistance to the herbicide bialophos. B. Construct pMBXO84 contains a seed-specific expression cassette, driven by the promoter from the soya bean oleosin isoform A gene, for expression of the CCP1 gene from Chlamydomonas reinhardtii. An expression cassette for the bar gene, driven by the CaMV35S promoter, imparts transgenic plants resistance to the herbicide bialophos.

### DETAILED DESCRIPTION OF THE INVENTION

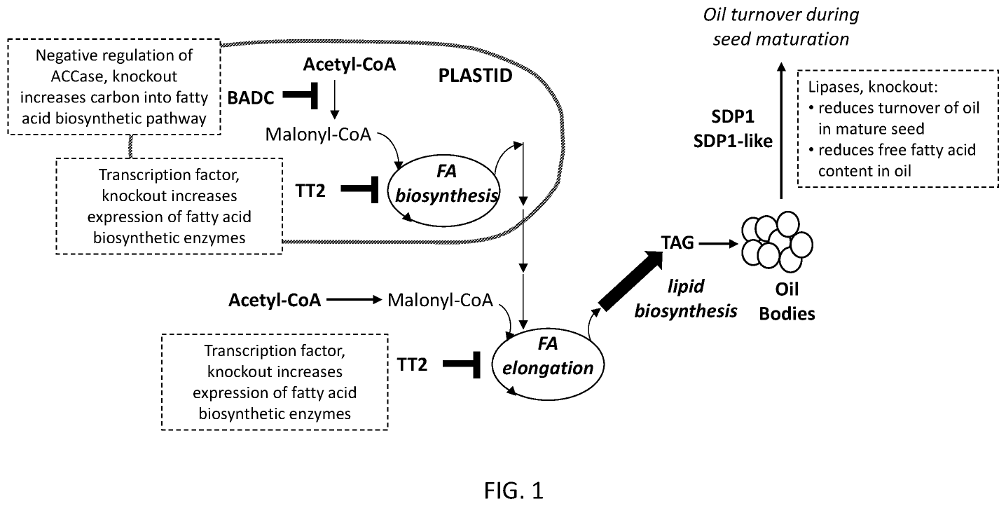

Herein we describe surprising improvements of TAG accumulation in plants by modulating the activity of multiple genes involved in fatty acid biosynthesis, TAG biosynthesis, and TAG degradation. Preferably these modifications to the activity of multiple genes are accomplished without introducing DNA sequences from a different species. Preferred methods for modulating the activity of the genes include genome editing and cis-genic approaches, including cis-genic systems expressing RNA inhibitors of expression of the target genes such as RNAi or anti-sense. As described herein we have focused our efforts on four gene targets, SDP1, SDP1-L, TT2, and BADC, that may be useful to increase TAG production in oilseeds. Where the products of each of these genes fit in oil metabolism are illustrated in FIG. 1.

We have identified full-length single gene homologs for SDP1, SDP1-like, TT2, and BADC proteins in Camelina sativa, canola, and soybean as targets for reducing their expression or activity using genome editing as a means to increase oil content while minimizing the reduction in seed yield seen by most researchers using other approaches. These oilseed crops have more complex genomes than the diploid genome of Arabidopsis, with multiple homeologs of each gene. Thus, for example, Camelina, an allohexaploid, has three homeologs of SDP1 genes, each present in two copies. As discussed below, using genome editing with the CRISPR/Cas9 system to knockout two copies of the three different SDP1 genes (six total copies) in Camelina proved very difficult, and typically we were only successful in inactivating the two copies of a single homeolog of SDP1. When stable homozygous plants with single homeolog knockouts were analyzed for seed yield and oil content, we determined that the oil content of the seed was not negatively affected, however quite surprisingly we found that the edited lines had a significantly higher seed yield contrary to all previous reports.

### I. DEFINITIONS

The following terms, unless otherwise indicated, will be understood to have the following meanings:

The term “plant” includes whole plant, mature plants, seeds, shoots and seedlings, and parts, propagation material, plant organ tissue, protoplasts, callus and other cultures, for example cell cultures, derived from plants belonging to the plant subkingdom Embryophyta, and all other species of groups of plant cells giving functional or structural units, also belonging to the plant subkingdom Embryophyta. The term “mature plants” refers to plants at any developmental stage beyond the seedling. The term “seedlings” refers to young, immature plants at an early developmental stage. The terms “crops” and “plants” are used interchangeably.

As used herein a “genetically modified plant” refers to non-naturally occurring plants or crops engineered as described throughout herein.

As used herein a “control plant” means a plant that has not been modified as described in the present disclosure to impart an enhanced trait or altered phenotype. A control plant is used to identify and select a modified plant that has an enhanced trait or altered phenotype. For instance, a control plant can be a plant that has not been modified or has not been genome edited to express or to inhibit its endogenous gene product. A suitable control plant can be a non-transgenic or non-edited plant of the parental line used to generate a transgenic plant, for example, a wild-type plant devoid of a recombinant DNA or a genome edit. A suitable control plant can also be a transgenic plant that contains recombinant DNA that imparts other traits, for example, a transgenic plant having enhanced herbicide tolerance. A suitable control plant can in some cases be a progeny of a hemizygous transgenic plant line that does not contain the recombinant DNA, known as a negative segregant, a null segregant, or a negative isogenic line.

As used herein the term “seed oil content” refers to amount of oil per mature seed weight and is typically expressed as a percentage.

As used herein the term “seed yield” refers to weight of seeds produced per plant and is typically expressed in grams per plant.

As used herein the term “oil yield” refers to weight of oil produced per plant and is typically expressed as grams per plant.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct,” which are used interchangeably, refers to any gene that is not a native gene, comprising regulatory and coding sequences that are not found together in nature. A “Cis-genic gene” is a chimeric gene where the DNA sequences making up the gene are from the same plant species or a sexually compatible plant species where the cis-genic gene is deployed in the same species from which the DNA sequences were obtained. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

As used herein the term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

As used herein “gene” includes protein coding regions of the specific genes and the regulatory sequences both 5′ and 3′ which control the expression of the gene.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for increased expression in a host cell, it is desirable to design the nucleic acid fragment such that its frequency of codon usage approaches the frequency of preferred codon usage of the host cell.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity). When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percent sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif).

As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

“Homeologs” are pluralities of genes (e.g. two, three, or more genes) that originated by speciation and were brought back together in the same genome by allopolyploidization (Glover et al., 2016, Trends Plant Sci., 21, 609).

“Polyploidy” is a heritable condition of an organism having more than two complete sets of chromosomes (Woodhouse et al., 2009, Nature Education, 2, 1). For example, a “tetraploid” has four sets of chromosomes. A “hexaploid” has six sets of chromosomes.

“Allopolyploidy” is a type of whole-genome duplication by hybridization followed by genome doubling (Glover et al., 2016). Allopolyploidy typically occurs between two related species, and results in the merging of the genomes of two divergent species into one genome. For example, an “allotetraploid” is an alloploid that has four sets of chromosomes. An “allohexaploid” is a hexaploid that has six sets of chromosomes.

“Autopolyploidy” is a type of whole-genome duplication based on doubling of a genome within one species.

“Diploidization” of a polyploid is a process that involves genomic reorganization, restructuring, and functional alternations in association with polyploidy, generally resulting in restoration of a secondary diploid-like behavior of a polyploid genome (del Pozo et al., 2015, Journal Experimental Botany, 66, 6991). Most polyploid plants have lost their polyploidy over time through diploidization (del Pozo et al., 2015).

### II. PREFERRED EMBODIMENTS

As noted above, a genetically modified plant that exhibits an increase in seed yield relative to a progenitor plant from which the genetically modified plant was derived is provided.

The genetically modified plant comprises a first homeolog of the SUGAR-DEPENDENT1 (SDP1) gene, occurring in its natural position within the genome of the genetically modified plant and being homozygous for a wild-type allele. The wild-type allele encodes an active SDP1 triacylglycerol lipase and is identical to an allele of the first homeolog of the SDP1 gene from the progenitor plant.

The genetically modified plant also comprises a second homeolog of the SDP1 gene, occurring in its natural position within the genome of the genetically modified plant and being homozygous for a mutant allele. The mutant allele does not encode an active SDP1 triacylglycerol lipase and includes one or more additions, deletions, or substitutions of one or more nucleotides relative to an allele of the second homeolog of the SDP1 gene from the progenitor plant.

In some embodiments, the genetically modified plant comprises the first homeolog and the second homeolog based on one or more of polyploidy, alloploidy, autoploidy, diploidization following polyploidy, diploidization following alloploidy, or diploidization following autoploidy. In some embodiments, the genetically modified plant is allotetetraploid, allohexaploid, or allooctoploid.

The genetically modified plant expresses about 20% to 80% of SDP1 triacylglycerol lipase activity in seeds relative to the progenitor. This is based on the plant not having a full complement of wild-type alleles of homeologs of SDP1, and particularly not having wild-type alleles of the second homeolog of SDP1. In some embodiments, the genetically modified plant expresses about 30% to 70% of SDP1 triacylglycerol lipase activity in seeds relative to the progenitor. For example, in some embodiments the genetically modified plant expresses about 30% to 40%, about 40% to 50%, about 50% to 60%, or about 60% to 70% of SDP1 triacylglycerol lipase activity in seeds relative to the progenitor. Also for example, in some embodiments the genetically modified plant expresses about 30% to 36%, about 45% to 55%, or about 63% to 70% of SDP1 triacylglycerol lipase activity in seeds relative to the progenitor.

The increase in seed yield is at least 10%. As noted above, surprisingly we found that the edited lines had a significantly higher seed yield. In some embodiments, the increase in seed yield is at least 20%. For example in some embodiments, the increase in seed yield is at least 25%, 30%, 35%, 40%, 45%, 50% or more.

As noted above, the first homeolog of the SDP1 gene is homozygous for the wild-type allele. In some embodiments, the genetically modified plant is homozygous for the wild-type allele based on including two identical copies of a wild-type allele. The identical wild-type alleles may be derived, for example, from a single wild-type allele of a progenitor plant. In some embodiments, the genetically modified plant is homozygous for the wild-type allele based on including a first wild-type allele and a second wild-type allele that are not identical to each other. The non-identical wild-type alleles may differ, for example, based on differences in the nucleotide sequences of the non-identical alleles that are sufficiently minor as to have no corresponding phenotype with respect to SDP1 triacylglycerol lipase activity.

As also noted above, the second homeolog of the SDP1 gene is homozygous for the mutant allele. In some embodiments, the genetically modified plant is homozygous for the mutant allele based on including two copies of the mutant allele that are identical. The identical mutant alleles may be based, for example, on breeding the genetically modified plant to homozygosity with respect to a particular mutant allele. In some embodiments, the genetically modified plant is homozygous for the mutant allele based on including a first mutant allele and a second mutant allele that are not identical to each other. The non-identical mutant alleles may differ, for example, based on having different additions, deletions, and/or substitutions of one or more nucleotides relative to each other, with the additions, deletions, and/or substitutions of each being sufficiently severe to cause a loss of function of the SDP1 triacylglycerol lipase encoded by each.

In some embodiments, the active SDP1 triacylglycerol lipase has a sequence that is at least 70% identical to one or more SEQ ID NO: 30, SEQ ID NO: 31, or SEQ ID NO: 32. In some embodiments, the active SDP1 triacylglycerol lipase has a sequence that comprises SEQ ID NO: 30, SEQ ID NO: 31, or SEQ ID NO: 32.

In some embodiments, the one or more additions, deletions, or substitutions of one or more nucleotides comprise one or more of a frameshift mutation, an active site mutation, a nonconservative substitution mutation, or an open-reading-frame deletion mutation in the mutant allele relative to the allele of the second homeolog of the SDP1 gene from the progenitor plant.

In some embodiments the genetically modified plant further comprises a third homeolog of the SDP1 gene occurring in its natural position within the genome of the genetically modified plant. In some of these embodiments, the third homeolog is homozygous for a wild-type allele. In some of these embodiments, the third homeolog is homozygous for a mutant allele. In some of these embodiments, the third homeolog is heterozygous for a wild-type allele and a mutant allele.

In some embodiments, the genetically modified plant further comprises: (a) a first homeolog of the SUGAR-DEPENDENT1-LIKE (SDP1-L) gene, occurring in its natural position within the genome of the genetically modified plant and being homozygous for a wild-type allele; and (b) a second homeolog of the SDP1-L gene, occurring in its natural position within the genome of the genetically modified plant and being homozygous for a mutant allele. In these embodiments, the wild-type allele encodes an active SDP1-L triacylglycerol lipase and is identical to an allele of the first homeolog of the SDP1-L gene from the progenitor plant. Also in these embodiments, the mutant allele does not encode an active SDP1-L triacylglycerol lipase and includes one or more additions, deletions, or substitutions of one or more nucleotides relative to an allele of the second homeolog of the SDP1-L gene from the progenitor plant.

In some embodiments, the genetically modified plant further comprises: (a) a first homeolog of the TRANSPARENT TESTA2 (TT2) gene, occurring in its natural position within the genome of the genetically modified plant and being homozygous for a wild-type allele; and (b) a second homeolog of the TT2 gene, occurring in its natural position within the genome of the genetically modified plant and being homozygous for a mutant allele. In these embodiments, the wild-type allele encodes an active TT2 transcription factor and is identical to an allele of the first homeolog of the TT2 gene from the progenitor plant. Also in these embodiments, the mutant allele does not encode an active TT2 transcription factor and includes one or more additions, deletions, or substitutions of one or more nucleotides relative to an allele of the second homeolog of the TT2 gene from the progenitor plant.

In some embodiments, the genetically modified plant is one or more of a Brassica species, Brassica napus, Brassica rapa, Brassica carinata, Brassica juncea, Camelina sativa, a Crambe species, a Jatropha species, pennycress, Ricinus communis, a Calendula species, a Cuphea species, Arabidopsis thaliana, maize, soybean, a Gossypium species, sunflower, palm, coconut, safflower, peanut, Sinapis alba, sugarcane, flax, or tobacco. In some embodiments, the genetically modified plant is Brassica napus, Brassica rapa, Brassica carinata, Brassica juncea, Camelina sativa, or soybean.

In some embodiments, the genetically modified plant is Camelina sativa. In some of these embodiments, the natural position of the second homeolog of the SDP1 gene is on chromosome 13 of Camelina sativa. Also in some of these embodiments, the allele of the second homeolog of the SDP1 gene from the progenitor plant encodes a protein that has a sequence comprising SEQ ID NO: 31. Also in some of these embodiments, the allele of the second homeolog of the SDP1 gene from the progenitor plant comprises SEQ ID NO: 2. Also in some of these embodiments, the genetically modified plant further comprises a third homeolog of the SDP1 gene occurring in its natural position within the genome of the genetically modified plant, wherein the third homeolog is homozygous for a wild-type allele.

### III. GENETIC MODIFICATION OF PLANTS

### Methods of Plant Transformation

Known transformations methods can be used to genetically modify a plant with respect to one or more gene sequences of the invention using transgenic, cis-genic, or genome editing methods.

### Vectors

Several plant transformation vector options are available, including those described in Gene Transfer to Plants, 1995, Potrykus et al., eds., Springer-Verlag Berlin Heidelberg New York, Transgenic Plants: A Production System for Industrial and Pharmaceutical Proteins, 1996, Owen et al., eds., John Wiley & Sons Ltd. Eng, and Methods in Plant Molecular Biology: A Laboratory Course Manual, 1995, Maliga et al., eds., Cold Spring Laboratory Press, New York. Plant transformation vectors generally include one or more coding sequences of interest under the transcriptional control of 5′ and 3′ regulatory sequences, including a promoter, a transcription termination and/or polyadenylation signal, and a selectable or screenable marker gene.

Many vectors are available for transformation using Agrobacterium tumefaciens. These typically carry at least one T-DNA sequence and include vectors such as pBIN19. Typical vectors suitable for Agrobacterium transformation include the binary vectors pCIB200 and pCIB2001, as well as the binary vector pCIB 10 and hygromycin selection derivatives thereof (see, for example, U.S. Pat. No. 5,639,949).

Transformation without the use of Agrobacterium tumefaciens circumvents the requirement for T-DNA sequences in the chosen transformation vector and consequently vectors lacking these sequences are utilized in addition to vectors such as the ones described above which contain T-DNA sequences. The choice of vector for transformation techniques that do not rely on Agrobacterium depends largely on the preferred selection for the species being transformed. Typical vectors suitable for non-Agrobacterium transformation include pCIB3064, pSOG 19, and pSOG35. (See, for example, U.S. Pat. No. 5,639,949). Alternatively, DNA fragments containing the transgene and the necessary regulatory elements for expression of the transgene can be excised from a plasmid and delivered to the plant cell using microprojectile bombardment-mediated, or alternatively, nanotube-mediated methods.

### Protocols

Transformation protocols as well as protocols for introducing nucleotide sequences into plants may vary depending on the type of plant or plant cell targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055; Zhao et al. WO US98/01268), direct gene transfer (Paszkowski et al. (1984) EMBO J. 3:2717-2722), and ballistic particle acceleration (see, for example, Sanford et al., U.S. Pat. No. 4,945,050; Tomes et al. (1995) Plant Cell, Tissue, and Organ Culture: Fundamental Methods, ed. Gamborg and Phillips (Springer-Verlag, Berlin); and McCabe et al. Biotechnology 6:923-926 (1988)). Also see Weissinger et al. Ann. Rev. Genet. 22:421-477 (1988); Sanford et al. Particulate Science and Technology 5:27-37 (1987) (onion); Christou et al. Plant Physiol. 87:671-674 (1988) (soybean); McCabe et al. (1988) BioTechnology 6:923-926 (soybean); Finer and McMullen In Vitro Cell Dev. Biol. 27P:175-182 (1991) (soybean); Singh et al. Theor. Appl. Genet. 96:319-324 (1998)(soybean); Dafta et al. (1990) Biotechnology 8:736-740 (rice); Klein et al. Proc. Natl. Acad. Sci. USA 85:4305-4309 (1988) (maize); Klein et al. Biotechnology 6:559-563 (1988) (maize); Tomes, U.S. Pat. No. 5,240,855; Buising et al., U.S. Pat. Nos. 5,322,783 and 5,324,646; Tomes et al. (1995) in Plant Cell, Tissue, and Organ Culture: Fundamental Methods, ed. Gamborg (Springer-Verlag, Berlin) (maize); Klein et al. Plant Physiol. 91:440-444 (1988) (maize); Fromm et al. Biotechnology 8:833-839 (1990) (maize); Hooykaas-Van Slogteren et al. Nature 311:763-764 (1984); Bowen et al., U.S. Pat. No. 5,736,369 (cereals); Bytebier et al. Proc. Natl. Acad. Sci. USA 84:5345-5349 (1987) (Liliaceae); De Wet et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler et al. Theor. Appl. Genet. 84:560-566 (1992) (whisker-mediated transformation); D'Halluin et al. Plant Cell 4:1495-1505 (1992) (electroporation); Li et al. Plant Cell Reports 12:250-255 (1993) and Christou and Ford Annals of Botany 75:407-413 (1995) (rice); Osjoda et al. Nature Biotechnology 14:745-750 (1996) (maize via Agrobacterium tumefaciens). References for protoplast transformation and/or gene gun for Agrisoma technology are described in WO 2010/037209. Methods for transforming plant protoplasts are available including transformation using polyethylene glycol (PEG), electroporation, and calcium phosphate precipitation (see for example Potrykus et al., 1985, Mol. Gen. Genet., 199, 183-188; Potrykus et al., 1985, Plant Molecular Biology Reporter, 3, 117-128). Methods for plant regeneration from protoplasts have also been described [Evans et al., in Handbook of Plant Cell Culture, Vol 1, (Macmillan Publishing Co., New York, 1983); Vasil, IK in Cell Culture and Somatic Cell Genetics (Academic, Oro, 1984)].

Transformation protocols as well as protocols for introducing nucleotide sequences into plants may vary depending on the type of plant or plant cell, i.e., monocot or dicot, targeted for transformation.

Suitable methods of introducing nucleotide sequences into plant cells and subsequent insertion into the plant genome are described in US 2010/0229256 A1 to Somleva & Ali and US 2012/0060413 to Somleva et al.

The transformed cells are grown into plants in accordance with conventional techniques (see, for example, McCormick et al., 1986, Plant Cell Rep. 5: 81-84). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Procedures for in planta transformation can be simple. Tissue culture manipulations and possible somaclonal variations are avoided and only a short time is required to obtain transgenic plants. However, the frequency of transformants in the progeny of such inoculated plants is relatively low and variable. At present, there are very few species that can be routinely transformed in the absence of a tissue culture-based regeneration system. Stable Arabidopsis transformants can be obtained by several in planta methods including vacuum infiltration (Clough & Bent, 1998, The Plant J. 16: 735-743), transformation of germinating seeds (Feldmann & Marks, 1987, Mol. Gen. Genet. 208: 1-9), floral dip (Clough and Bent, 1998, Plant J. 16: 735-743), and floral spray (Chung et al., 2000, Transgenic Res. 9: 471-476). Other plants that have successfully been transformed by in planta methods include rapeseed and radish (vacuum infiltration, Ian and Hong, 2001, Transgenic Res., 10: 363-371; Desfeux et al., 2000, Plant Physiol. 123: 895-904), Medicago truncatula (vacuum infiltration, Trieu et al., 2000, Plant J. 22: 531-541), camelina (floral dip, WO/2009/117555 to Nguyen et al.), and wheat (floral dip, Zale et al., 2009, Plant Cell Rep. 28: 903-913). In planta methods have also been used for transformation of germ cells in maize (pollen, Wang et al. 2001, Acta Botanica Sin., 43, 275-279; Zhang et al., 2005, Euphytica, 144, 11-22; pistils, Chumakov et al. 2006, Russian J. Genetics, 42, 893-897; Mamontova et al. 2010, Russian J. Genetics, 46, 501-504) and Sorghum (pollen, Wang et al. 2007, Biotechnol. Appl. Biochem., 48, 79-83).

### Selection

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the DNA construct for introducing the targeted insertion of the DNA sequence elements producing the desired level of desired polypeptide(s) in the desired tissue and cellular location.

The cells that have been transformed may be grown into plants in accordance with conventional techniques (see, for example, McCormick et al. Plant Cell Reports 5:81-84(1986)). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

Transgenic plants can be produced using conventional techniques to express any genes of interest in plants or plant cells (Methods in Molecular Biology, 2005, vol. 286, Transgenic Plants: Methods and Protocols, Pena L., ed., Humana Press, Inc. Totowa, N.J.; Shyamkumar Barampuram and Zhanyuan J. Zhang, Recent Advances in Plant Transformation, in James A. Birchler (ed.), Plant Chromosome Engineering: Methods and Protocols, Methods in Molecular Biology, vol. 701, Springer Science+Business Media). Typically, gene transfer, or transformation, is carried out using explants capable of regeneration to produce complete, fertile plants. Generally, a DNA or an RNA molecule to be introduced into the organism is part of a transformation vector. A large number of such vector systems known in the art may be used, such as plasmids. The components of the expression system can be modified, e.g., to increase expression of the introduced nucleic acids. For example, truncated sequences, nucleotide substitutions or other modifications may be employed. Expression systems known in the art may be used to transform virtually any plant cell under suitable conditions. A transgene comprising a DNA molecule encoding a gene of interest is preferably stably transformed and integrated into the genome of the host cells. Transformed cells are preferably regenerated into whole fertile plants. Detailed description of transformation techniques are within the knowledge of those skilled in the art.

Plant promoters can be selected to control the expression of the transgene in different plant tissues or organelles for all of which methods are known to those skilled in the art (Gasser & Fraley, 1989, Science 244: 1293-1299). In one embodiment, promoters are selected from those of eukaryotic or synthetic origin that are known to yield high levels of expression in plants and algae. In a preferred embodiment, promoters are selected from those that are known to provide high levels of expression in monocots.

Constitutive promoters include, for example, the core promoter of the Rsyn7 promoter and other constitutive promoters disclosed in WO 99/43838 and U.S. Pat. No. 6,072,050, the core CaMV 35S promoter (Odell et al., 1985, Nature 313: 810-812), rice actin (McElroy et al., 1990, Plant Cell 2: 163-171), ubiquitin (Christensen et al., 1989, Plant Mol. Biol. 12: 619-632; Christensen et al., 1992, Plant Mol. Biol. 18: 675-689), pEMU (Last et al., 1991, Theor. Appl. Genet. 81: 581-588), MAS (Velten et al., 1984, EMBO J. 3: 2723-2730), and ALS promoter (U.S. Pat. No. 5,659,026). Other constitutive promoters are described in U.S. Pat. Nos. 5,608,149; 5,608,144; 5,604,121; 5,569,597; 5,466,785; 5,399,680; 5,268,463; and 5,608,142.

“Tissue-preferred” promoters can be used to target gene expression within a particular tissue. Compared to chemically inducible systems, developmentally and spatially regulated stimuli are less dependent on penetration of external factors into plant cells. Tissue-preferred promoters include those described by Van Ex et al., 2009, Plant Cell Rep. 28: 1509-1520; Yamamoto et al., 1997, Plant J. 12: 255-265; Kawamata et al., 1997, Plant Cell Physiol. 38: 792-803; Hansen et al., 1997, Mol. Gen. Genet. 254: 337-343; Russell et al., 1997, Transgenic Res. 6: 157-168; Rinehart et al., 1996, Plant Physiol. 112: 1331-1341; Van Camp et al., 1996, Plant Physiol. 112: 525-535; Canevascini et al., 1996, Plant Physiol. 112: 513-524; Yamamoto et al., 1994, Plant Cell Physiol. 35: 773-778; Lam, 1994, Results Probl. Cell Differ. 20: 181-196, Orozco et al., 1993, Plant Mol. Biol. 23: 1129-1138; Matsuoka et al., 1993, Proc. Natl. Acad. Sci. USA 90: 9586-9590, and Guevara-Garcia et al., 1993, Plant J. 4: 495-505. Such promoters can be modified, if necessary, for weak expression.

Any of the described promoters can be used to control the expression of one or more of the genes of the invention, their homologs and/or orthologs as well as any other genes of interest in a defined spatiotemporal manner.

### Expression Cassettes

Nucleic acid sequences intended for expression in transgenic plants are first assembled in expression cassettes behind a suitable promoter active in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described infra.

A variety of transcriptional terminators are available for use in expression cassettes. These are responsible for the termination of transcription beyond the transgene and the correct polyadenylation of the transcripts. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

Individual plants within a population of transgenic plants that express a recombinant gene(s) may have different levels of gene expression. The variable gene expression is due to multiple factors including multiple copies of the recombinant gene, chromatin effects, and gene suppression. Accordingly, a phenotype of the transgenic plant may be measured as a percentage of individual plants within a population. The yield of a plant can be measured simply by weighing. The yield of seed from a plant can also be determined by weighing. The increase in seed weight from a plant can be due to a number of factors, an increase in the number or size of the seed pods, an increase in the number of seed or an increase in the number of seed per plant. In the laboratory or greenhouse seed yield is usually reported as the weight of seed produced per plant and in a commercial crop production setting yield is usually expressed as weight per acre or weight per hectare.

A recombinant DNA construct including a plant-expressible gene or other DNA of interest is inserted into the genome of a plant by a suitable method. Suitable methods include, for example, Agrobacterium tumefaciens-mediated DNA transfer, direct DNA transfer, liposome-mediated DNA transfer, electroporation, co-cultivation, diffusion, particle bombardment, microinjection, gene gun, calcium phosphate coprecipitation, viral vectors, and other techniques. Suitable plant transformation vectors include those derived from a Ti plasmid of Agrobacterium tumefaciens. In addition to plant transformation vectors derived from the Ti or root-inducing (Ri) plasmids of Agrobacterium, alternative methods can be used to insert DNA constructs into plant cells. A transgenic plant can be produced by selection of transformed seeds or by selection of transformed plant cells and subsequent regeneration.

In one embodiment, the transgenic plants are grown (e.g., on soil) and harvested. In one embodiment, above ground tissue is harvested separately from below ground tissue. Suitable above ground tissues include shoots, stems, leaves, flowers, grain, and seed. Exemplary below ground tissues include roots and root hairs. In one embodiment, whole plants are harvested and the above ground tissue is subsequently separated from the below ground tissue.

Genetic constructs may encode a selectable marker to enable selection of transformation events. There are many methods that have been described for the selection of transformed plants [for review see (Miki et al., Journal of Biotechnology, 2004, 107, 193-232) and references incorporated within]. Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298, Waldron et al., (1985), Plant Mol Biol, 5:103-108; Zhijian et al., (1995), Plant Sci, 108:219-227), the bar gene encoding resistance to phosphinothricin (U.S. Pat. No. 5,276,268), the expression of aminoglycoside 3′-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684). Other suitable selectable markers include, but are not limited to, genes encoding resistance to chloramphenicol (Herrera Estrella et al., (1983), EMBO J, 2:987-992), methotrexate (Herrera Estrella et al., (1983), Nature, 303:209-213; Meijer et al, (1991), Plant Mol Biol, 16:807-820); streptomycin (Jones et al., (1987), Mol Gen Genet, 210:86-91); bleomycin (Hille et al., (1990), Plant Mol Biol, 7:171-176); sulfonamide (Guerineau et al., (1990), Plant Mol Biol, 15:127-136); bromoxynil (Stalker et al., (1988), Science, 242:419-423); glyphosate (Shaw et al., (1986), Science, 233:478-481); phosphinothricin (DeBlock et al., (1987), EMBO J, 6:2513-2518).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson et al., Nat Biotechnol, 2004, 22, 455-8). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants.

Methods for positive selection using sorbitol dehydrogenase to convert sorbitol to fructose for plant growth have also been described (WO 2010/102293). Screenable marker genes include the beta-glucuronidase gene (Jefferson et al., 1987, EMBO J. 6: 3901-3907; U.S. Pat. No. 5,268,463) and native or modified green fluorescent protein gene (Cubitt et al., 1995, Trends Biochem. Sci. 20: 448-455; Pan et al., 1996, Plant Physiol. 112: 893-900).

Transformation events can also be selected through visualization of fluorescent proteins such as the fluorescent proteins from the nonbioluminescent Anthozoa species which include DsRed, a red fluorescent protein from the Discosoma genus of coral (Matz et al. (1999), Nat Biotechnol 17: 969-73). An improved version of the DsRed protein has been developed (Bevis and Glick (2002), Nat Biotech 20: 83-87) for reducing aggregation of the protein.

Visual selection can also be performed with the yellow fluorescent proteins (YFP) including the variant with accelerated maturation of the signal (Nagai, T. et al. (2002), Nat Biotech 20: 87-90), the blue fluorescent protein, the cyan fluorescent protein, and the green fluorescent protein (Sheen et al. (1995), Plant J 8: 777-84; Davis and Vierstra (1998), Plant Molecular Biology 36: 521-528). A summary of fluorescent proteins can be found in Tzfira et al. (Tzfira et al. (2005), Plant Molecular Biology 57: 503-516) and Verkhusha and Lukyanov (Verkhusha, V. V. and K. A. Lukyanov (2004), Nat Biotech 22: 289-296) whose references are incorporated in entirety. Improved versions of many of the fluorescent proteins have been made for various applications. It will be apparent to those skilled in the art how to use the improved versions of these proteins or combinations of these proteins for selection of transformants.

The plants modified for enhanced performance may be combined or stacked with input traits by crossing or plant breeding. Useful input traits include herbicide resistance and insect tolerance, for example a plant that is tolerant to the herbicide glyphosate and that produces the Bacillus thuringiensis (BT) toxin. Glyphosate is a herbicide that prevents the production of aromatic amino acids in plants by inhibiting the enzyme 5-enolpyruvylshikimate-3-phosphate synthase (EPSP synthase). The overexpression of EPSP synthase in a crop of interest allows the application of glyphosate as a weed killer without killing the modified plant (Suh, et al., J. M Plant Mol. Biol. 1993, 22, 195-205). BT toxin is a protein that is lethal to many insects providing the plant that produces it protection against pests (Barton, et al. Plant Physiol. 1987, 85, 1103-1109). Other useful herbicide tolerance traits include but are not limited to tolerance to Dicamba by expression of the dicamba monoxygenase gene (Behrens et al, 2007, Science, 316, 1185), tolerance to 2,4-D and 2,4-D choline by expression of a bacterial aad-1 gene that encodes for an aryloxyalkanoate dioxygenase enzyme (Wright et al., Proceedings of the National Academy of Sciences, 2010, 107, 20240), glufosinate tolerance by expression of the bialophos resistance gene (bar) or the pat gene encoding the enzyme phosphinotricin acetyl transferase (Droge et al., Planta, 1992, 187, 142), as well as genes encoding a modified 4-hydroxyphenylpyruvate dioxygenase (HPPD) that provides tolerance to the herbicides mesotrione, isoxaflutole, and tembotrione (Siehl et al., Plant Physiol, 2014, 166, 1162). The plants modified for enhanced yield by reducing the expression of the transcription factor genes or transcription factor gene combinations may be combined or stacked with other genes which improve plant performance.

### Genome Editing

Genome editing can also be used to accomplish genetic modification of plants according to the invention. An advantage of using genome editing technologies is that the regulatory body in the United States views genome editing as an advanced plant breeding tool and may not regulate the technologies. Recent advances in genome editing technologies provide an opportunity to precisely remove genes, edit control sequences, introduce frame shift mutations, etc., to significantly alter the expression levels of targeted genes and/or the activities of the proteins encoded thereby. Plants engineered using this approach may be defined as non-regulated by USDA-APHIS providing the opportunity to continually improve the plants. Given the timelines and costs associated with achieving regulatory approval for transgenic plants this approach enables a single regulatory filing instead of having to continuously file for regulatory approval for each subsequent genetic modification to improve the plants.

Genome editing can be accomplished by using Clustered Regularly Interspaced Short Palindromic Repeats (CRISPR/Cas9) or CRISPR/Cpf1. The use of this technology in genome editing is well described in the art (Fauser et al, 2014, The Plant Journal, Vol 79, p 348-359; Belhaj, K., 2013, Plant Methods 9, 39; Khandagale & Nadal, 2016, Plant Biotechnol Rep 10, 327). In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). At least two classes (Class I and II) and six types (Types I-VI) of Cas proteins have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR/Cas is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the Type II CRISPR-Cas system, and a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms.

For applications in eukaryotic organisms, codon optimized versions of Cas9, which is originally from the bacterium Streptococcus pyogenes, have been used. The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Cas9 expression plasmids for use in the methods of the invention can be constructed as described in the art.

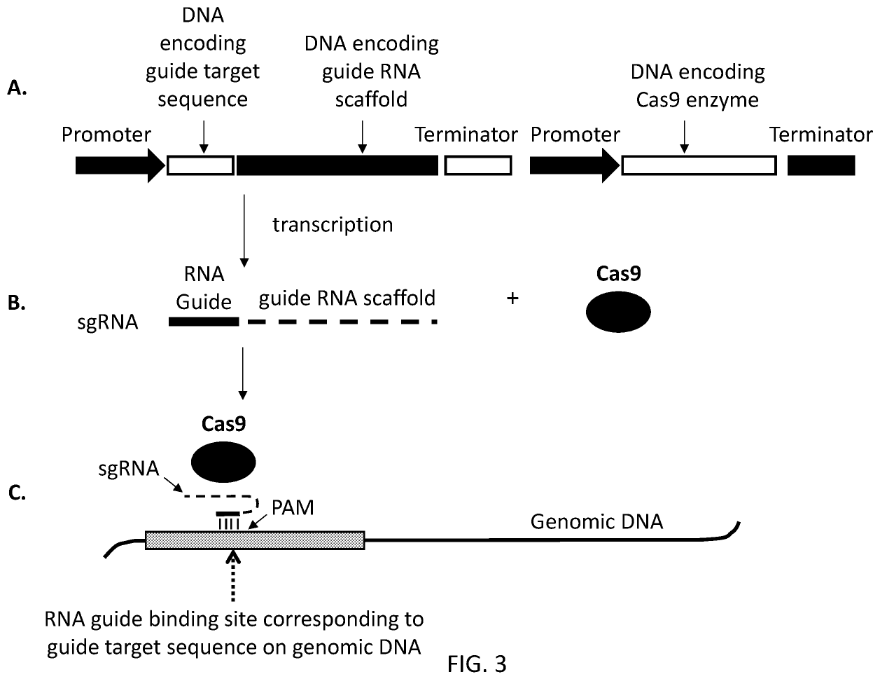

The Cas9 enzyme and sgRNA can introduced to the cells to be edited using multiple methods. Genetic transformation of an expression construct encoding the sgRNA and the Cas9 enzyme (FIG. 3) can be used to edit the cells. Subsequent removal of the transgenes encoding the sgRNA and the Cas9 enzyme can be achieved through segregation yielding plants with only the genome edit. Alternatively, the sgRNA can be synthesized in vitro and introduced into cells, often in the form of Ribonucleoprotein complexes (RNPs) that contain Cas9 protein to promote cleavage of the target genomic DNA at the “guide target sequence”.

Various other methods can be used for gene editing, by using transcription activator-like effector nucleases (TALENs), clustered Regularly Interspaced Short Palindromic Repeats (CRISPR/Cas9) or zinc-finger nucleases (ZFN) techniques (as described in Belhaj et al, 2013, Plant Methods, vol 9, p 39, Chen et al, 2014 Methods Volume 69, Issue 1, p 2-8).

### EXAMPLES

### Example 1. Identification of the Camelina Orthologs of the Arabidopsis SUGAR-DEPENDENT1 (sdp1) and SUGAR-DEPENDENT1-Like (sdp1-Like) Genes

Triacylglycerol (TAG) content in oil seeds is highest during the late seed maturation phase, but in many species declines during the following desiccation phase. This loss can account for about 10% of the maximum oil content in Brassica napus seeds grown in the greenhouse or in the field (Chia et al., 2005, Journal of Experimental Botany, 56, 1285; Kelly et al., 2013, Plant Biotechnology Journal, 11, 355). Oil catabolism is initiated by triacylglycerol lipases that hydrolyze fatty acids off the glycerol backbone for subsequent conversion into sugars or amino acids via β-oxidation, glyoxylate cycle and gluconeogenesis. Two oil body-associated triacylglycerol lipases SUGAR-DEPENDENT1 (SDP1) and SUGAR-DEPENDENT1-LIKE (SDP1-like) have been identified in Arabidopsis thaliana. Both enzymes together contribute over 95% of triacylglycerol lipase activity during seed germination (Eastmond, 2006, Plant Cell, 18, 665).

The sdp1 and sdp1-like genes were selected for editing in Camelina sativa to reduce the turnover of TAGs that occurs in mature seeds, both to prevent yield loss and to prevent the undesirable accumulation of free fatty acids in oil. The Arabidopsis thaliana genes encoding SDP1 and SDP1-Like are listed in TABLE 1. GenBank was searched for genes annotated as sdp1 or sdp1-like in the Camelina sativa DH55 genome and by using the Genbank BLAST search tool using the Arabidopsis SDP1 and SDP1-like proteins as queries. Eight sequences were identified and are listed in TABLE 2. Two of these sequences (SEQ ID NO: 2, SEQ ID NO: 3) were annotated in Genbank as triacylglycerol SDP1 lipases. The remaining six sequences were annotated as triacylglycerol lipase SDP1-like, SDP1L, or SDP1L-like. One sequence was incomplete and was eliminated from future analyses.

TABLE 1
Arabidopsis genes encoding SDP1 and SDP1-like

| | | | | Protein |
|---|---|---|---|---|
| | | | | Length |
| | | Genbank | Protein ID | (amino |
| Gene | Gene IDs | Description | (SEQ ID NO:) | acids) |
| sdp1 | At5g04040 | Patatin-like | NP_196024.1 | 825 |
| | NM_120486.7 | phospholipase | (SEQ ID NO: 28) | |
| | | family protein | | |
| | | (SDP1) | | |
| sdp1- | At3g57140 | SDP1-Like | AAO64904.1 | 801 |
| like | BT005969.1 | sugar-dependent | (SEQ ID NO: 29) | |
| | | 1-like protein | | |

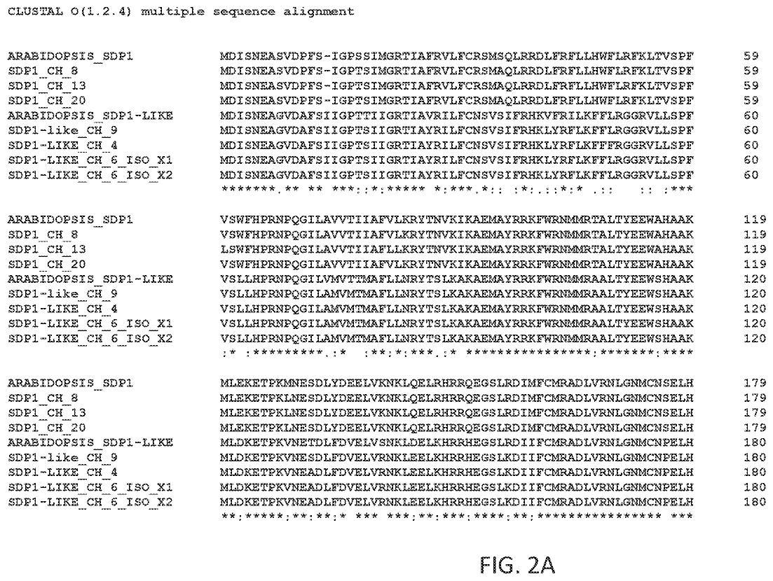

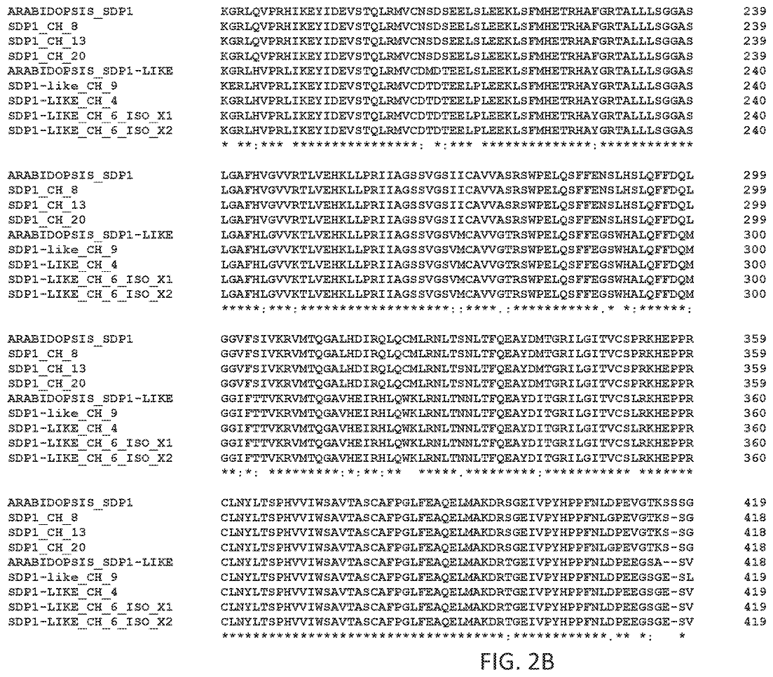

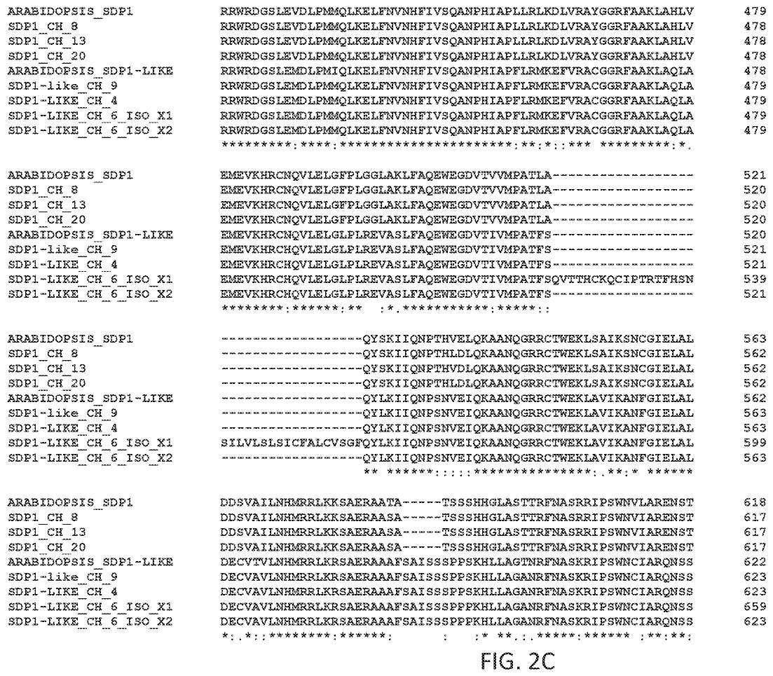

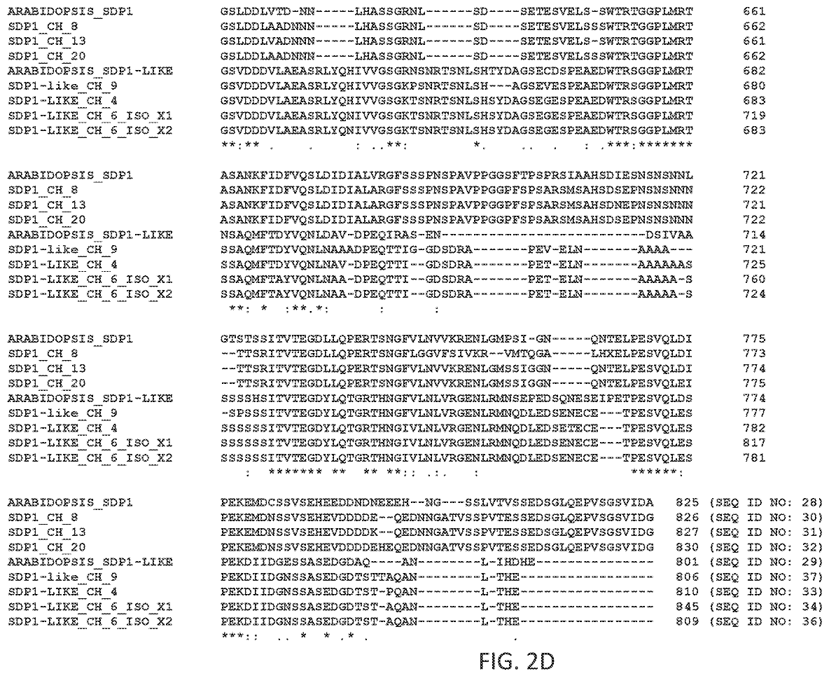

Since Camelina is an allohexaploid containing three subgenomes (for review see Malik et al., 2018, Plant Cell Rep, 37, 1367), three copies of each gene are expected. A combination of syntenic analysis and sequence alignment was used to identify the three homeologous copies of SDP1 and SDP1-like in the allohexaploid Camelina sativa genome. FIG. 2A-D shows a Clustal O multiple sequence alignment of the Arabidopsis SDP1 and SDP1-like protein and the Camelina orthologs found through this analysis. The three copies of SDP1 were identified on chromosome 8 (XM_010425338.2, SEQ ID NO: 1), chromosome 13 (XM_010453992.2, SEQ ID NO: 2;), and chromosome 20 (XM_010492596.2, SEQ ID NO: 3). The Arabidopsis SDP1 protein closely aligns with the identified Camelina SDP1 proteins on Chromosomes 8, 13 and 20 (FIG. 2A-D). The copy on chromosome 8 had been previously annotated as an SDP1-like lipase in Genbank (TABLE 2). The three copies of the sdp1-like genes were found in the Camelina genome on chromosome 4 (XM_010506334.2, SEQ ID NO: 7), chromosome 6 (XM_010518019.2, SEQ ID NO: 8), and chromosome 9 (XM_010429278.2, SEQ ID NO: 9). A protein isoform for the gene on chromosome 6 was also predicted (SEQ ID NO: 35) in GenBank which was larger by 36 amino acid residues due to an extra internal sequence. The Arabidopsis SDP1-like protein closely aligns with the identified Camelina SDP1-like proteins on Chromosomes 4, 6 and 9 (FIG. 2A-D).

TABLE 2
SDP1 and SDP1-like sequences identified in Camelina sativa

| | | | | | Protein |
|---|---|---|---|---|---|
| | | | | | Length |
| Gene IDs | Gene | Genbank | | Protein ID | (amino |
| (SEQ ID NO:) | Name² | Description | Location¹ | (SEQ ID NO:) | acids) |
| LOC104708721 | SDP1 | Triacylglycerol | Ch 8 | XP_010423640.1 | 826 |
| (SEQ ID NO: 1) | | lipase SDP1-like | | (SEQ ID NO: 30) | |
| LOC104734421 | SDP1 | Triacylglycerol | Ch 13 | XP_010452294.1 | 827 |
| (SEQ ID NO: 2) | | lipase SDP1 | | (SEQ ID NO: 31) | |
| LOC104768592 | SDP1 | Triacylglycerol | Ch 20 | XP_010490898.2 | 830 |
| (SEQ ID NO: 3) | | lipase SDP1 | | (SEQ ID NO: 32) | |
| | | | | XP_019097840.1 | |
| | | | | (2 accessions have | |
| | | | | same protein sequence) | |
| LOC104774729 | | Triacylglycerol | Incomplete | | 126 |
| | | lipase SDP1-like | on 3′ end, | | |
| | | | on unplaced | | |
| | | | scaffold | | |
| LOC104781618 | SDP1-like | Triacylglycerol | Ch 4 | XP_010504636.1 | 810 |
| (SEQ ID NO: 7) | | lipase SDP1L | | (SEQ ID NO: 33) | |
| LOC104792005 | SDP1-like | Triacylglycerol | Ch 6 | XP_010516321.1 | 845 |
| (SEQ ID NO: 8) | | lipase SDP1L- | | (SEQ ID NO: 34) | |
| | | like isoform X1 | | | |
| LOC104792005 | SDP1-like | Triacylglycerol | Ch 6 | XP_010516322.1 | 809 |
| (SEQ ID NO: 35) | | lipase SDP1L- | | (SEQ ID NO: 36) | |
| | | like isoform X2 | | | |
| LOC104712383 | SDP1-like | Triacylglycerol | Ch 9 | XP_010427580.1 | 806 |
| (SEQ ID NO: 9) | | lipase SDP1L- | | (SEQ ID NO: 37) | |
| | | like | | | |

¹Abbreviation: Ch, chromosome.
²In the present work, the annotation of the gene as SDP1 or SDP1-like was based on sequence similarity analysis, as well as syntenic analysis.

### Example 2. Genome Editing of the Camelina sativa SUGAR DEPENDENT1 (sdp1) Gene Encoding a Triacylglycerol Lipase

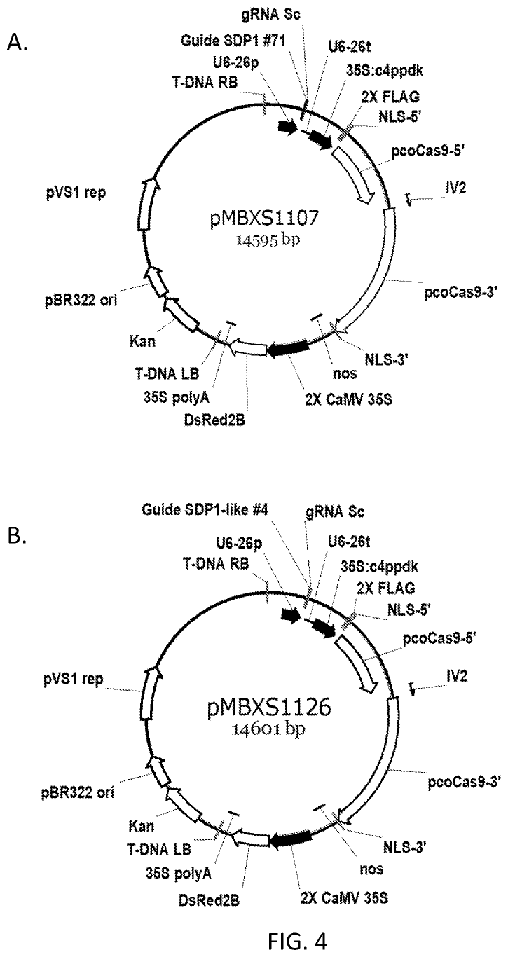

The large seeded C. sativa germplasm 10CS0043 (abbreviated WT43) that was obtained from a breeding program at Agriculture and Agri-Food Canada was used for genome editing of the sdp1 gene target. To create mutations in the sdp1 genes, genetic constructs were designed that would generate a single guide RNA (sgRNA) within the plant cell and produce a functional Cas9 enzyme molecule. FIG. 3 shows the genetic elements required for editing and how they interact with genomic DNA to achieve an edit. Genetic construct pMBXS1107 (FIG. 4(A), SEQ ID NO: 5), a binary vector containing expression cassettes to produce an sgRNA to target the sdp1 genes, a plant-codon optimized Cas9 (pcoCas9, Li et al., 2013. Nature Biotechnology, 31, 688), and the DsRed gene, which encodes a red fluorescent protein from the Discosoma genus of coral (Matz et al., 1999, Nat Biotechnol 17, 969), was constructed. DsRed expression can be used to distinguish transformed T1 seeds from untransformed seeds using a fluorescence microscope or by shining light of the correct wavelength on the seeds and viewing through the appropriate filter. Construct pMBXS1107 (FIG. 4(A)) was designed with the Guide sequence SDP1-#71 (SEQ ID NO: 4; TABLE 3) fused to DNA encoding the RNA scaffold (FIG. 3) to allow formation of the functional sgRNA for editing all three copies of the sdp1 gene. Guide SDP1-#71 was designed to target all three homeologs of the sdp1 gene in Camelina WT43 germplasm. In prior work in our laboratories, it was found that using one construct to edit the three homeologous copies of genes on three different Camelina chromosomes could be accomplished routinely if the genes were amenable to editing.

TABLE 3
Guide sequences for editing the sdp1 gene.

| | | Guide target | | |
|---|---|---|---|---|
| Guide name | Target Gene¹ | Strand | Guide target sequence (5′ to 3′) | PAM |
| SDP1-#71 | sdp1 Chr 8 | + | GACATGACAGGAAGGATACT | TGG |
| | (XM_010425338.2, | | (SEQ ID NO: 4) | |
| | SEQ ID NO. 1) | | | |
| | sdp1 Chr 13 | | | |
| | (XM_010453992.2, | | | |
| | SEQ ID NO. 2) | | | |
| | sdp1 Chr 20 | | | |
| | (XM_010492596.2, | | | |
| | SEQ ID NO. 3) | | | |

Construct pMBXS1107 (FIG. 4(A)) was transformed into Camelina using Agrobacterium-mediated floral dip transformation procedures (Lu and Kang, 2008, Plant Cell Rep, 27, 273) as follows.

In preparation for plant transformation experiments, seeds of Camelina sativa germplasm 10CS0043 (abbreviated WT43, obtained from Agriculture and Agri-Food Canada) were sown directly into 4 inch (10 cm) pots filled with soil in the greenhouse. Growth conditions were maintained at 24° C. during the day and 18° C. during the night. Plants were grown until flowering. Plants with a number of unopened flower buds were used in “floral dip” transformations.

Agrobacterium strain GV3101 (pMP90) was transformed with plasmid pMBXS1107 using electroporation. A single colony of GV3101 (pMP90) containing the construct of interest was obtained from a freshly streaked plate and was inoculated into 5 mL LB medium. After overnight growth at 28° C., 2 mL of culture was transferred to a 500-mL flask containing 300 mL of LB and incubated overnight at 28° C. Cells were pelleted by centrifugation (4,000 rpm, 20 min), and diluted to an OD600 of ˜0.8-1.0 with infiltration medium containing 5% sucrose and 0.05% (v/v) Silwet-L77 (Lehle Seeds, Round Rock, Tex., USA). Plants of Camelina sativa germplasm WT43 were transformed by “floral dip” using the pMBXS1107 transformation construct as follows. Pots containing plants at the flowering stage were placed inside a 460 mm height vacuum desiccator (Bel-Art, Pequannock, N.J., USA). Inflorescences were immersed into the Agrobacterium inoculum contained in a 500-ml beaker. A vacuum (85 kPa) was applied and held for 5 min. Plants were removed from the desiccator and were covered with plastic bags in the dark for 24 h at room temperature. Plants were removed from the bags and returned to normal growth conditions within the greenhouse for seed formation (T1 generation of seed).

T1 seeds were screened by monitoring the expression of DsRed, a marker on the T-DNA in plasmid vector pMBXS1107 (FIG. 4(A)) allowing the identification of transgenic seeds. DsRed expression in the seed was visualized by fluorescent microscopy using a Nikon AZ100 microscope with a TRITC-HQ(RHOD)2 filter module (HQ545/30X, Q570LP, HQ610/75M) as previously described (Malik et al., 2015, Plant Biotechnology Journal, 13, 675).

T1 generation DsRed+ seeds were selected and planted in soil. Plantlets were grown in a greenhouse under supplemental lighting. Tissue was harvested from plants with 3-4 leaves and amplicon sequencing was used to identify edited lines. Amplicon sequencing allows a survey of the different types of edits in a plant (i.e. deletions, insertions) as well as a determination of the number of alleles of the target gene that are edited. A fee for service provider was used to perform amplicon sequencing work. The analysis of amplicon sequencing data from wild-type WT43 plants showed that each sdp1 allele was represented in almost equal numbers (i.e. approximately 33% of sequences correspond to each allele, TABLE 4). The slight deviation from the expected 33% for each allele may be due to a slight bias during PCR for the alleles present on different chromosomes.

TABLE 4
Summary of Amplicon sequencing reads for
sdp1 alleles in WT43 control line.

| | Total | Chromosome | Chromosome | Chromosome |
|---|---|---|---|---|
| Plant | reads | 8 (% reads) | 13 (% reads) | 20 (% reads) |
| WT43 | 100% | 33.8 | 30.0% | 36.2% |
| control | | | | |

Amplicon sequencing data for the T1 lines transformed with pMBXS1107 showed edits mostly in the form of 1 to 6 base pair deletions or single base pair insertions in the sdp1 gene. The T1 generation line with the highest percentage of edited alleles contained 13.86% editing (line NS56, TABLE 5).

TABLE 5
Summary of Amplicon sequencing reads for sdp1 gene edits in
select representative T1 lines transformed with pMBXS1107.

| | | % edited | % edited | % edited |
|---|---|---|---|---|
| | % edited | reads, sdp1 | reads, sdp1 | reads, sdp1 |
| | reads, all | Chromosome | Chromosome | Chromosome |
| Plant | sdp1 alleles¹ | 8² | 13² | 20² |
| NS37 | 12.23% | 4.58% | 4.20% | 3.45% |
| NS56 | 13.86% | 4.63% | 3.69% | 5.54% |
| NS62 | 1.75% | 0.63% | 0.32% | 0.80% |
| NS63 | 0% | 0% | 0% | 0% |

¹For complete editing of all three gene copies in allohexaploid Camelina, a value of 100% is expected.
²For a completely edited chromosomal allele, a value of approximately 33% is expected.

After confirmation of edits in T1 lines, select lines were advanced by growing the plants to produce T2 generation seed. The segregation of the transformed T-DNA sequences (includes expression cassettes for the DsRed marker gene, Cas9 enzyme, and sgRNA) from the edited line was monitored with loss of the visible DsRed marker in T2 seeds and amplicon sequencing verification that the edit was retained in the T2 DsRed- lines. At this point in line development, edits were not yet homozygous and often required at least one additional cycle of breeding to achieve a homozygous edit.

T2 lines were allowed to produce T3 seeds that were planted in the greenhouse to generate T3 lines. Tissue from T3 lines was harvested and edits were characterized by amplicon sequencing. In the T3 generation homozygous edits were obtained, however the maximum number of edited alleles in lines was two (TABLE 6) despite having observed heterozygous edits in all three sdp1 gene copies in the T1 generation (TABLE 5). T3 lines with homozygous editing in the SDP1 alleles on chromosome 13 or chromosome 8, as well as lines with homozygous editing in SDP1 alleles on chromosomes 13 and 20 were identified (TABLE 6). The sequence of the edited regions is shown for select lines in TABLE 6.

TABLE 6
Summary of edits in best homozygous T3 lines edited in the SDP1 gene

| | Summary of Edits in | | | | |
|---|---|---|---|---|---|
| | SDP1 gene | | | | |
| Line | Ch 8 | Ch 13 | Ch 20 | Sequence of edited region¹ | |
| Wild-type | — | — | — | Ch 8 | AAGGAT-ACTTGG (SEQ ID NO: 110) |
| | | | | Ch 13 | AAGGAT-ACTTGG (SEQ ID NO: 110) |
| | | | | Ch 20 | AAGGAT-ACTTGG (SEQ ID NO: 110) |
| NS14 Line 17-0781 | — | X | — | Ch 8 | AAGGAT-ACTTGG (SEQ ID NO: 110) |
| | | | | Ch 13 | AAGGATTACTTGG (SEQ ID NO: 111) |
| | | | | Ch 20 | AAGGAT-ACTTGG (SEQ ID NO: 110) |
| NS14 Line 17-0783 | — | X | — | Ch 8 | AAGGAT-ACTTGG (SEQ ID NO: 110) |
| | | | | Ch 13 | AAGGATTACTTGG (SEQ ID NO: 111) |
| | | | | Ch 20 | AAGGAT-ACTTGG (SEQ ID NO: 110) |
| NS37 line 17-0831 | X | — | — | Ch 8 | AAGGATTACTTGG (SEQ ID NO: 111) |
| | | | | Ch 13 | AAGGAT-ACTTGG (SEQ ID NO: 110) |
| | | | | Ch 20 | AAGGAT-ACTTGG (SEQ ID NO: 110) |
| NS37 line 17-0836 | X | — | — | Ch 8 | AAGGATTACTTGG (SEQ ID NO: 111) |
| | | | | Ch 13 | AAGGAT-ACTTGG (SEQ ID NO: 110) |
| | | | | Ch 20 | AAGGAT-ACTTGG (SEQ ID NO: 110) |
| NS37 line 17-0865 | — | X | X | Ch 8 | AAGGAT-ACTTGG (SEQ ID NO: 110) |
| | | | | Ch 13 | AAGGATTACTTGG (SEQ ID NO: 111) |
| | | | | Ch 20 | AAGGATTACTTGG (SEQ ID NO: 111) |

¹Edited bases, either insertions, or substitutions, are shown in bold, underlined letters.
²Symbol “X” denotes complete editing of the chromosomal allele of the gene and “-” denotes the wild-type sequence of the chromosomal allele of the gene.

T4 seeds were harvested from T3 homozygous edited plants and a total of eleven lines with homozygous editing were compared to wild-type control plants for seed yield and oil content. Results from the best plants are summarized in TABLE 7. The highest yielding plants were those that contained edits in only the SDP1 gene located on chromosome 13 (NS14 lines, TABLE 7) leaving the copies on chromosomes 8 and 20 intact. Seed yields in these plants increased by up to 39% over the wild-type unedited control plants (TABLE 7). These edited lines had similar seed oil content as wild-type. The increased seed yield increased the total oil produced per plant by up to 40% compared to the control (TABLE 7).

TABLE 7
Summary of T4 seed production from best T3 lines edited in SDP1 gene

| | Summary of | | | | | T4 seed | % | | |
|---|---|---|---|---|---|---|---|---|---|
| | Edits in | | | T4 | | oil | increase | Oil | % |
| | SDP1 gene³ | | | seed | | content | in seed | produced | increase |
| | Ch | Ch | Ch | yield | % increase | (% seed | oil | (g per T3 | in oil |
| Line | 8 | 13 | 20 | (g) | T4 seed yield | weight) | content | plant)² | produced |
| Wild- | — | — | — | 8 ± 2 | — | 34.3 ± 2.8 | — | 2.74 | — |
| type¹ | | | | | | | | | |
| NS14 | — | X | — | 11 | 37.5 | 34.7 | 1.2 | 3.82 | 39 |
| Line | | | | | | | | | |
| 17- | | | | | | | | | |
| 0781 | | | | | | | | | |
| NS14 | — | X | — | 11.1 | 38.8 | 34.6 | 0.9 | 3.84 | 40 |
| Line | | | | | | | | | |
| 17- | | | | | | | | | |
| 0783 | | | | | | | | | |
| NS37 | X | — | — | 8.9 | 11.3 | 36.03 | 5.04 | 3.21 | 17.2 |
| Line | | | | | | | | | |
| 17- | | | | | | | | | |
| 0831 | | | | | | | | | |
| NS37 | X | — | — | 10.1 | 26.3 | 34.7 | 1.2 | 3.42 | 25 |
| Line | | | | | | | | | |
| 17- | | | | | | | | | |
| 0836 | | | | | | | | | |
| NS37 | — | X | X | 6.01 | −24.8 | 31.2 | −9.0 | 1.88 | −31.4 |
| line | | | | | | | | | |
| 17- | | | | | | | | | |
| 0865 | | | | | | | | | |

¹Wild-type data is from the average of six wild-type control plants.
²Oil per plant for each line (calculated from seed yield and seed oil content).
³Symbol “X” denotes complete editing of the chromosomal allele of the gene and denotes the wild-type sequence of the chromosomal allele of the gene.

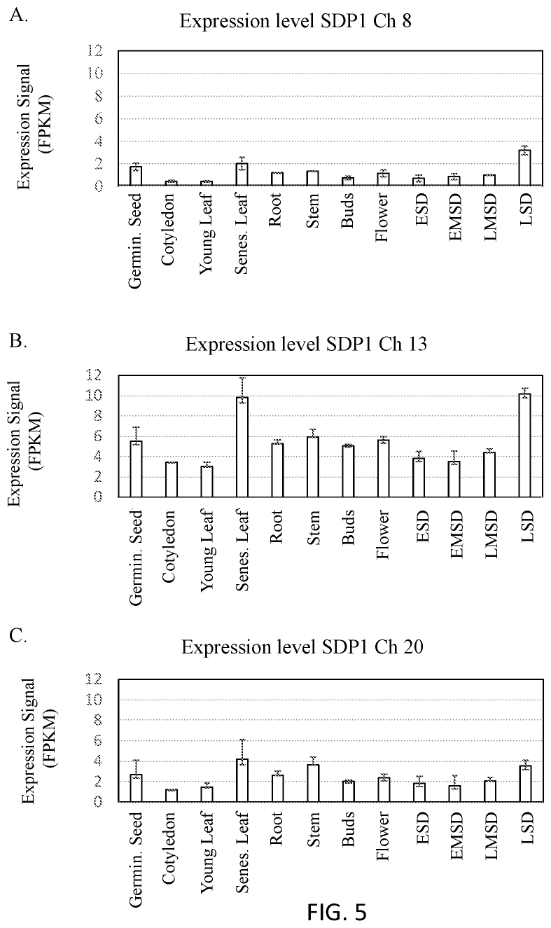

The results in TABLE 7 suggest that it is difficult to edit all three homeologs of sdp1 in Camelina. To examine the expression profiles of the three different homeologs, the Camelina eFP Browser (website: //bar.utoronto.ca/efp_camelina/cgi-bin/efpWeb.cgi) was used. The expression profile of the sdp1 genes on Chromosomes 8, 13, and 20 suggests that the gene is expressed to some degree in many different tissue types (FIG. 5). Interestingly, the gene on Chromosome 13, which gave the highest seed yield upon editing, is the highest expressed homeolog according to the Camelina eFP browser.

### Example 3. Genome Editing of the Camelina sativa SUGAR DEPENDENT1 Like (sdp1-Like) Gene Encoding a Triacylglycerol Lipase

The gene encoding SUGAR-DEPENDENT1-LIKE (SDP1-like), another oil body associated TAG lipase, was edited using Agrobacterium-mediated transformation of pMBXS1126 (FIG. 4(B), SEQ ID NO: 6), a genetic construct that contains expression cassettes for sgRNA to target all three copies of the sdp1-like gene in Camelina, as well as expression cassettes for the Cas9 enzyme and the DsRed visual marker protein. Three copies of the sdp1-like genes were found in the Camelina genome on chromosome 4 (SEQ ID NO: 7), chromosome 6 (SEQ ID NO: 8), and chromosome 9 (SEQ ID NO: 9). Construct pMBXS1126 (FIG. 4(B), SEQ ID NO: 6) was designed with the Guide sequence SDP1-like #4 (SEQ ID NO: 10, TABLE 8) fused to DNA encoding the RNA scaffold (FIG. 3) to allow formation of the functional sgRNA for editing all three copies of the sdp1-like gene.

TABLE 8
Guide sequences for editing the SDP1-like gene.

| Guide | | Guide target | | |
|---|---|---|---|---|
| name | Target Gene | Strand | Guide target sequence (5′ to 3′) | PAM |
| SDPl-like | SDP1-Like Chr4 | - | GTTCTACCGATTATAGACGT | GGG |
| #4 | (XM_010506334.2, | | (SEQ ID NO: 10) | |
| | SEQ ID NO. 7) | | | |
| | SDP1-Like Chr6 | | | |
| | (XM_010518019.2, | | | |
| | SEQ ID NO. 8) | | | |
| | SDP1-Like Chr9 | | | |
| | (XM_010429278.2, | | | |
| | SEQ ID NO. 9) | | | |

Camelina WT43 was transformed with pMBXS1126, using the Camelina transformation procedures described above, and 48 T1 lines were obtained. Analyses of amplicon sequencing data showed that edits obtained were mostly in the form of insertions of 1 base pair or deletions of 1-30 base pairs. Four T1 lines with a high percentage of edits (38 to 79% total editing in all sdp1-like alleles) were obtained for this target and advanced to the T3 generation in the greenhouse to remove the T-DNA insert by segregation by monitoring loss of the visual DsRed marker in seeds.

Forty DsRed negative T2 seeds from each of three T1 lines [OA05 (79% editing), OA07 (55% editing) and OA09 (63% editing)] were planted in the greenhouse with wild-type controls for line advancement purposes. T2 plants were genotyped for editing and the most promising plants were analyzed by Amplicon sequencing. Although we did not obtain homozygous edited lines for all three copies of the SDP1-like gene in the T2 generation, several transgene-free progenies of OA05 showed a high percentage of editing. One T2 line, 16-4076, with 77.4% total editing amongst all sdp1-like alleles, showed complete editing of the sdp1-like alleles on chromosome 4 and 6 and partial editing (heterozygous) of the allele on chromosome 9. Other transgene-free lines (16-4073, 16-4083, and 16-4092) showed complete editing of sdp1-like alleles on chromosomes 4 and 6 and 0% editing on chromosome 9. Edited plants showed vegetative and floral characteristics similar to the wild-type controls. A few plants had larger siliques/pods during T3 seed set.

T3 seeds were planted in the greenhouse and five T3 transgene-free plants derived from T2 plant 16-4076 showed 100% editing in all three alleles by amplicon sequencing. Two of these T3 generation 100% edited lines, lines 17-0607 and 17-0609 (TABLE 9), were propagated further for yield assessment. T3 lines 17-0607 and 17-0609 differ in the nature of their edits with 17-0607 having homozygous and 17-069 having heterozygous edits in chromosomes 6 and 9 in terms of sequence (TABLE 9). These lines were allowed to set seed which was sown in the greenhouse. Progeny (six T4 plants each) of the two edited lines as well as wild-type controls were grown in the greenhouse and amplicon sequencing was performed. The nature of edits in the six T4 progeny plants of 17-0607 was similar to the parental T3 plant 17-0607 showing stable inheritance of all edits (TABLE 9). The T4 progeny of T3 line 17-0609 showed 5 plants with one type of editing (an insertion of nucleotide ‘A’ in the sdp1-like alleles on chromosomes 6 and 9 and an insertion of ‘T’ in the allele on chromosome 4) and one plant with a different editing pattern (an insertion of ‘T’ in all three alleles of sdp1-like gene). This result was expected since T3 plant 17-0609 was heterozygous for editing in the alleles on chromosome 6 and 9 and therefore showed segregation in the nature of edits in the T4 plant population (TABLE 9). The editing in the form of a 1 base pair insertion in all of the edited lines in TABLE 9 will produce a truncated polypeptide leading to a non-functional SDP1-like protein.

TABLE 9
Summary of edits in best lines edited in the sdp1-like gene

| | | Summary of Edits | | | | |
|---|---|---|---|---|---|---|
| | | in SDP1 gene³ | | | | |
| Line | Generation | Ch 4 | Ch 6 | Ch 9 | Sequence of edited region² | |
| Wild- | — | — | — | — | Ch 4 | CCCACG-TC |
| type | | | | | Ch 6 | CCCACG-TC |
| | | | | | Ch 9 | CCTACG-TC |
| 17-0607 | T3 | X | X | X | Ch 4 | CCCACGTTC |
| | | | | | Ch 6 | CCCACGATC |
| | | | | | Ch 9 | CCTACGATC |
| 17-0609 | T3 | X | X | X | Ch 4 | CCCACGTTC |
| | | | | | Ch 61 | CCCACGTTC or |
| | | | | | | CCCACGATC |
| | | | | | Ch 91 | CCTACGTTC or |
| | | | | | | CCTACGATC |
| Progeny | T4 | X | X | X | Ch 4 | CCCACGTTC |
| of line | (6 plants tested; 6 with | | | | Ch 6 | CCCACGATC |
| 17-0607 | identical homozygous | | | | Ch 9 | CCTACGATC |
| | edits) | | | | | |
| Progeny | T4 | X | X | X | Ch 4 | CCCACGTTC |
| 17-0609 | (6 plants tested, 1 with | | | | Ch 6 | CCCACGTTC |
| of line | following homozygous | | | | Ch 9 | CCTACGTTC |
| | edits) | | | | | |
| | T4 | X | X | X | Ch 4 | CCCACGTTC |
| | (6 plants tested, 5 with | | | | Ch 6 | CCCACGATC |
| | following homozygous | | | | Ch 9 | CCTACGATC |
| | edits) | | | | | |

¹Amplicon sequencing reads showed two kinds of edits on chromosome which segregated in the next generation.
²Bold underlined letters indicate the edit in the sequence.
³Symbol “X” denotes complete editing of the chromosomal allele of the gene and “-” denotes the wild-type sequence of the chromosomal allele of the gene.

The two groups of T4 edited lines (progeny of lines 17-0607 and 17-0609, TABLE 9) displayed similar growth characteristics and height as the wild-type plants during the rosette, bolting, and flowering stages of plant development. The wild-type plants were taller than the edited plants by the end of flowering, and remained taller during seed filling and maturation. On average the plants of the two edited lines were 3-4 cm shorter than the wild-type plants as determined by height measurements of mature plants. This difference in height was statistically significant in the progeny of line 17-0609 as compared to the wild-type plants. Also, on average the edited plants flowered 1-2 days earlier than the wild-type but the difference was not statistically significant.

The average T4 seed yield of plants of the two edited lines, 14.4 g for T3 line 17-0609 and 14.2 g for T3 line 17-0607, was similar to those of the wild-type control plants that yielded an average of 13.4 g seeds (TABLE 10). Although a small increase of average seed yield of 1 g for line 17-0609 over the wild-type seed yield was recorded, this difference was not statistically significant (TABLE 10). Measurements of 1000 seed weights were performed with three replicate seed samples from each plant. The average 1000 T4 seed weights from T3 plants of the two lines, 17-0609 and 17-0607, were 4% and 7.9% higher than that of the wild-type plants. The increase in 1000 seed weights of 7.9% in plants of line 17-0607 as compared to the wild-type plants was statistically significant.

TABLE 10
Summary of T4 seed production from best T3 lines edited in sdp1-like gene

| | Summary of edits in | | | | | % increase T4 |
|---|---|---|---|---|---|---|
| | sdp1-like gene³ | | | | Average T4 1000 | weights |
| Line | Ch 4 | Ch 6 | Ch 9 | T4 seed yield (g) | seed weights (mg)² | 1000 seed |
| Wild-type | — | — | — | 13.4 ± 4.08¹ | 1,145 ± 76.99 | — |
| 17-0609 | X | X | X | 14.4 | 1,190.6 ± 99.43 | 4.0% |
| 17-0607 | X | X | X | 14.2 | 1,236.1 ± 93.34 | 7.9%* |

¹Wild-type data is from the average of six wild-type control plants.
²Performed with three replicated seed samples from each plant.
*Statistically significant compared to wild-type plants.
³Symbol “X” denotes complete editing of the chromosomal allele of the gene anddenotes the wild-type sequence of the chromosomal allele of the gene.

### Example 4. Multiplex Editing to Created Stacked Edits of SUGAR-DEPENDENT1 (sdp1), SUGAR-DEPENDENT1 Like (sdp1-Like), and TESTA2 (tt2) for Increased Oil and Seed Yield

The objective of this work was to use multiplex genome editing to combine, or stack, more than one edit of interest to obtain an additive yield effect. Targets for the first round of multiplex editing included sdp1, sdp1-like, and tt2. The tt2 gene encodes TRANSPARENT TESTA2, a transcription factor that coordinates the gene expression of enzymes for the proanthocyanidins in the seed coat and fatty acid biosynthesis in the embryo (Chen et al., Plant Physiol, 2012, 160, 1023; Wang et al., Plant J., 2014, 77, 757). The sdp1 and sdp1-like genes were previously described in Examples 1-3. While TT2 activates the biosynthetic pathway for proanthocyanidins in the seed coat, it represses the expression of the fatty acid biosynthetic pathway enzymes in the embryo by inhibiting the activity of the transcription factor FUSCA3. In tt2 Arabidopsis thaliana null mutants, expression of FUSCA3 is increased and leads to an increase in fatty acid biosynthesis in the seed embryo (Chen et al., Plant Physiol, 2012, 160, 1023). Arabidopsis mutants of tt2 lack the dark brown color of the condensed tannins (oxidized PAs) in the maternal seed coat (Wang et al., Plant J., 2014, 77, 757). The objective of the present research was to determine if stacking edits of sdp1, sdp1-like, and tt2 would provide a benefit for seed oil content and/or seed yield.

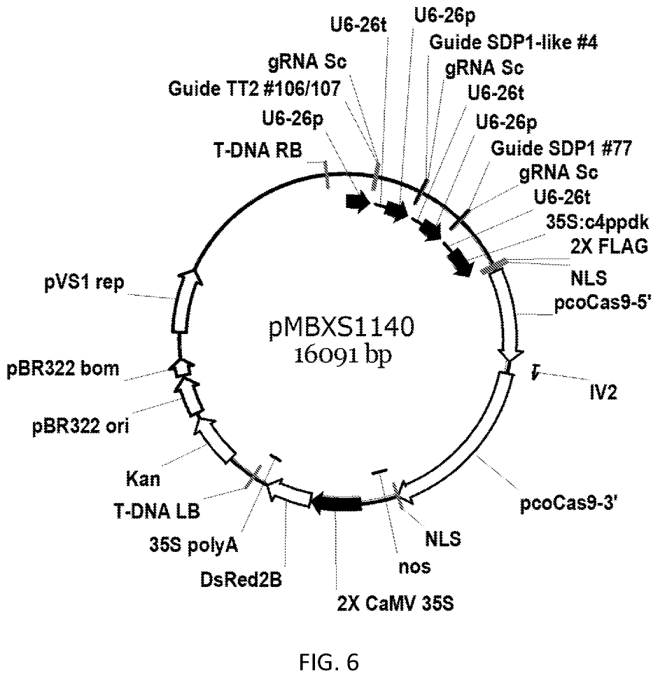

Three copies of the tt2 gene were identified in Camelina, one on chromosome 10 (SEQ ID NO: 11), one on chromosome 11 (SEQ ID NO: 12), and one on chromosome 12 (SEQ ID NO: 13) (TABLE 11). Genetic construct pMBXS1140 (FIG. 6, SEQ ID NO: 16) was designed with three separate expression cassettes for the Guide sequences shown in TABLE 11 to target editing of all three copies of the sdp1, sdp1-like, and tt2 genes. In pMBXS1140, each of these Guides are fused to DNA encoding the RNA scaffold (FIG. 3) to allow formation of the functional sgRNA for target specific editing. Construct pMBXS1140 also contains an expression cassette for Cas9 and an expression cassette for DsRed. Construct pMBXS1140 was transformed into Camelina using the procedures described above and 44 T1 lines were obtained. Amplicon sequencing of select T1 lines showed editing for all the three gene targets (TABLE 12).

TABLE 11
Guide sequences for multiplex editing of the sdp1, sdp1-like, and tt2 genes in
Camelina.

| Guide | | Guide target | | |
|---|---|---|---|---|
| name | Target Gene | Strand | Guide target sequence (5′ to 3′) | PAM |
| SDP1 #77 | sdp1 Chr 8 | + | CAAGAAAGCATGAACCTCCT | CGG |
| | (XM_010425338.2, | | (SEQ ID NO: 14) | |
| | SEQ ID NO. 1) | | | |
| | sdp1 Chr 13 | | | |
| | (XM_010453992.2, | | | |
| | SEQ ID NO. 2) | | | |
| | sdp1 Chr 20 | | | |
| | (XM_010492596.2, | | | |
| | SEQ ID NO. 3) | | | |
| SDP1- | SDP1-Like Chr 4 | - | GTTCTACCGATTATAGACGT | GGG |
| like #4 | (XM_010506334.2, | | (SEQ ID NO: 10) | |
| | SEQ ID NO. 7) | | | |
| | SDP1-Like Chr 6 | | | |
| | (XM_010518019.2, | | | |
| | SEQ ID NO. 8) | | | |
| | SDP1-Like Chr 9 | | | |
| | (XM_010429278.2, | | | |
| | SEQ ID NO. 9) | | | |
| TT2 | tt2 Chr 10 | + | GATGGTCGTTGATAGCTGGG | AGG |
| #106/107 | (XM_010438372.2, | | (SEQ ID NO: 15) | |
| | SEQ ID NO: 11) | | | |
| | tt2 Chr 11 | | | |
| | (XM_010442433.2, | | | |
| | SEQ ID NO: 12) | | | |
| | tt2 Chr 12 | | | |
| | (XM_010452083.2, | | | |
| | SEQ ID NO: 13) | | | |

As expected, amplicon sequencing data showed different editing efficiency for the three genes in the edited lines obtained from the pMBXS1140 transformation. The highest editing was observed in event OG31 T1 line 17-0309 with 69% total editing of the sdp1 gene, >99% editing in the sdp1-like gene, and 92% editing in the tt2 gene targets (TABLE 12) . Event OG15 T1 line 17-0293 showed editing of 49% in the sdp1 gene, 84.7% in the sdp1-like gene, and 46% editing in the tt2 gene target. Since all nine alleles of the three genes were edited in these two T1 lines, they were advanced to produce T2 seed.

TABLE 12
Summary of percent editing from amplicon sequencing data for eleven T1 lines transformed
with pMBXSl 140.

| | | | Dsred | sdp1 | sdp1-like | tt2 |
|---|---|---|---|---|---|---|
| ID | Event code | Generation | marker | % editing¹ | % editing¹ | % editing¹ |
| 17-0285 | OG07 | T1 | positive | 18.3% | 69.7% | 20.0% |
| 17-0287 | OG09 | T1 | positive | 23% | 63.6% | 18.1% |
| 17-0292 | OG14 | T1 | positive | 7% | 32% | 4.4% |
| 17-0293 | OG15 | T1 | positive | 49% | 84.7% | 46.4% |
| 17-0295 | OG17 | T1 | positive | 42% | 77.4% | 57.1% |
| 17-0305 | OG27 | T1 | positive | 16% | 54.8% | 16.8% |
| 17-0307 | OG29 | T1 | positive | 35% | 66.3% | 56.3% |
| 17-0308 | OG30 | T1 | positive | 40% | 58.6% | 41.8% |
| 17-0309 | OG31 | T1 | positive | 69% | 99.7% | 91.8% |
| 17-0315 | OG37 | T1 | positive | 11% | 56.2% | 13.1% |
| 17-0317 | OG39 | T1 | positive | 35% | 79.3% | 46.8% |

¹1% editing indicates the sum of total editing of all three alleles of a gene.

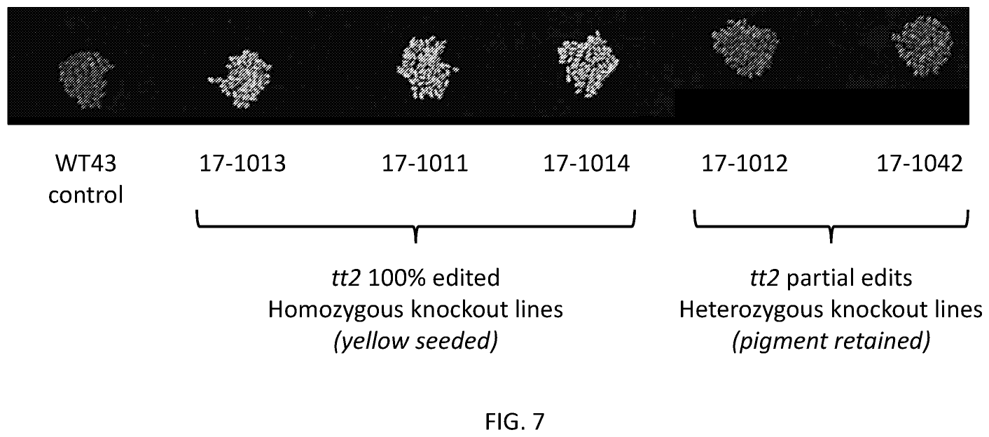

T2 plants were generated and amplicon sequencing was performed on select T2 plants. T2 transgene-free plants of the OG31 line, identified by loss of DsRed expression, were isolated that showed high editing in the sdp1, sdp1-like, and tt2 targets with some plants possessing 100% editing of tt2 or sdp1-like (TABLE 13). T3 seeds were harvested from the T2 plants and analyzed for seed yield and oil content. Lines with 100% editing in the tt2 gene showed no pigmentation in the seed coat due to the loss of the dark brown color of the condensed tannins (oxidized proanthocyanidins) in the maternal seed coat (FIG. 7). This phenotype is characteristic of plants containing an inactive tt2 protein and is observed in tt2 mutants of Arabidopsis (Wang et al., Plant J., 2014, 77, 757). This phenotype can give an important visual distinction to track a commercial line of edited seed.

TABLE 13
Summary of editing of sdp1, sdp1-like, and tt2 gene targets in DsRed negative
T2 plants generated from multiplex editing construct pMBXSl 140.

| | | SDP1 | | | | SDPl-like | | | | TT2 | | |
|---|---|---|---|---|---|---|---|---|---|---|---|---|
| | | % editing on | | | | % editing on | | | | % editing on | | |
| | SDP1 | individual | | | SDP1-L | individual | | | | individual | | |
| | % | chromosomes² | | | % | chromosomes² | | | TT2 | chromosomes² | | |
| Line | total | Ch | Ch | Ch | total | Ch | Ch | Ch | % total | Ch | Ch | Ch |
| ID | editing¹ | 13 | 20 | 8 | editing¹ | 4 | 6 | 9 | editing¹ | 10 | 11 | 12 |
| 17- | 83 | 33 | 34 | 16 | 80 | 20 | 29 | 31 | 100 | 33 | 34 | 33 |
| 1013 | | | | | | | | | | | | |
| 17- | 39 | 37 | 0 | 2 | 61 | 29 | 32 | 0 | 100 | 36 | 34 | 30 |
| 1011 | | | | | | | | | | | | |
| 17- | 65 | 32 | 17 | 16 | 60 | 31 | 29 | 0 | 100 | 34 | 35 | 32 |
| 1014 | | | | | | | | | | | | |
| 17- | 34 | 32 | 0 | 2 | 100 | 31 | 29 | 40 | 66 | 34 | 31 | 1 |
| 1012 | | | | | | | | | | | | |
| 17- | 81 | 18 | 38 | 26 | 100 | 30 | 31 | 39 | 50 | 34 | 1 | 14 |
| 1042 | | | | | | | | | | | | |

¹For complete editing of all three gene copies in a lohexaploid Camelina, a value of 100% is expected,
²For a completely edited chromosomal allele, a va lue of approximately 33% is expected.

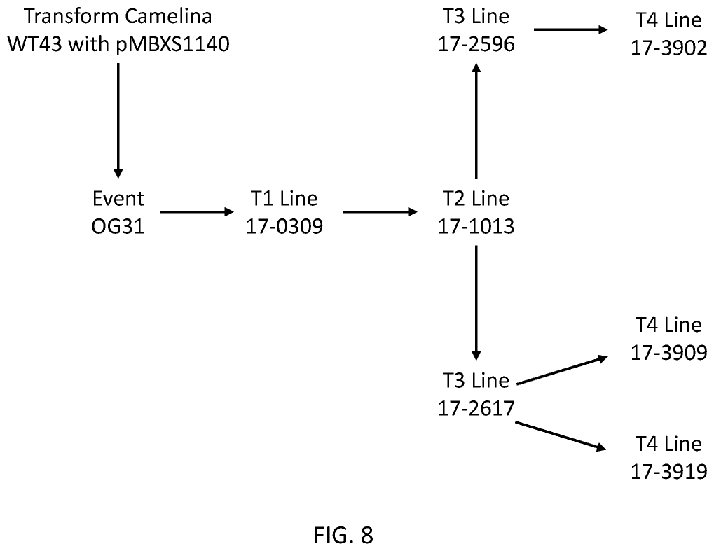

T2 line 17-1013 was chosen for advancement to generate stable homozygous edited lines as illustrated in FIG. 8. The advancement of seeds from select lines of the two sets was prioritized based on the nature of edits. T3 lines 17-2596 and 17-2617, progeny of line T1 line 17-0309 (FIG. 8), were completely homozygous for edits in two genes (sdp1-like and tt2 in 17-2596, sdp1 and tt2 in 17-2617) and heterozygous for editing for one allele of the third gene (sdp1 in 17-2596 and sdp1-like in 17-2617) and were categorized as higher priority lines. These T3 edited plants showed normal phenotype similar to the wild-type plants during the growth cycle. All lines with 100% editing in tt2 displayed a non-pigmented seed coat phenotype, such as shown for earlier generation lines in FIG. 7. Several T4 progeny plants of line 17-2596 and 17-2617 (FIG. 8) were grown out in a randomized complete block design and subsequent amplicon sequencing of these lines showed that they segregated in different patterns as expected from Mendelian segregation. The stably edited lines 17-3902, 17-3909 and 17-3919 were selected for further study (FIG. 8). Line 17-3919 is the only plant that showed 100% editing in sdp1, sdp1-like and tt2 genes. This low segregation ratio (1 out of 24 plants screened) suggests that it is difficult to generate homozygous and complete editing in the two lipases (sdp1 and sdp1-like genes) unless a large number of plants are screened, as was done in this experiment.

Seed yield, and oil content data of T5 seeds harvested from the lines 17-3902, 17-3909 and 17-3919 (FIG. 8) are shown in TABLE 14. Some of the stable edited lines showed a very high increase of milligrams of oil produced per individual seed (up to a 38% increase for line 17-3909 compared to the control wild-type, TABLE 14). This data showed that the designed edits are indeed increasing oil content in an individual seed. However, the lines with the highest increase in milligrams of oil produced per individual seed (e.g. Line 17-3909) also have a lower number of total harvested seeds per plant. This observed yield drag upon increasing oil content suggests that there may not be enough carbon or reducing power available in the plant to both significantly increase oil content and produce a normal amount of seeds. The plants producing a low seed number also tend to flower longer than wild-type controls. This suggests that the plant can sense that it has not produced the typical number of seeds and thus extends its reproductive phase in an attempt to produce more seed. To correct this observed yield drag and achieve the full benefit of increased oil production per seed, additional gene targets may need to be added to the lines edited in sdp1, sdp1-like, and tt2.

TABLE 14
Seed yield and oil content for T5 seed of select edited lines.

| | | | | | | | | | | % change | | | | % |
|---|---|---|---|---|---|---|---|---|---|---|---|---|---|---|
| | Edits in plant¹ | | | | | | | | | mgs of oil | % | % change | % | change |
| | sdp1-like | | | sdp1 | | | tt2 | | | per | change | individual | change | in oil |
| T4 | Ch | Ch | Ch | Ch | Ch | Ch | Ch | Ch | Ch | individual | seed oil | seed | in # of | per |
| Lines | 4 | 6 | 9 | 13 | 20 | 8 | 10 | 11 | 12 | seed | content | weight | seeds | plant |
| 17- | X | X | X | X | X | — | X | X | X | +12% | +9% | +1% | −4% | +5% |
| 3902 | | | | | | | | | | | | | | |
| 17- | X | X | — | X | X | X | X | X | X | +38% | +5% | +17% | −19% | −15% |
| 3909 | | | | | | | | | | | | | | |
| 17- | X | X | X | X | X | X | X | X | X | +34% | +6% | +9% | −29% | −26% |
| 3919 | | | | | | | | | | | | | | |
| Wild- | — | — | — | — | — | — | — | — | — | 0% | 0% | 0% | 0% | 0% |
| type | | | | | | | | | | | | | | |

¹Symbol ″X″ denotes complete editing of the chromosomal allele of t ie gene and denotes the
wild-type sequence of the chromosomal allele of the gene.

To determine the carbon partitioning between the seed oil and seed protein of the edited lines, one gram T5 seed samples obtained from T4 lines 17-3902 and 17-3909 (FIG. 8) were submitted for protein analysis to determine if protein content is altered with increased oil. Protein content was determined by a contract vendor using the AOCS Official Method Ba 4e-93, a generic method applicable to determining crude protein in oilseed meals and oilseeds (website: //www.aocs.org/attain-lab-services/methods/methods/method-detail?productId=111449). The protein content did not significantly change in the lines measured compared to control lines of wild-type WT43 lines (TABLE 15). Line 17-3919 was not analyzed for protein content due to limited seeds remaining for this line.

TABLE 15
Protein content in T5 seeds of edited lines.

| | | | | Protein content |
|---|---|---|---|---|
| | Edits in plant¹ | | | (% crude protein |
| Lines | sdp1-like | sdp1 | tt2 | of seed weight) |
| 17-3902 | X X X | X X _{—} | X X X | 29.8 |
| 17-3909 | X X _{—} | X X X | X X X | 32.8 |
| 17-3919 | X X X | X X X | X X X | * |
| WT43 | _ _ _{—} | _ _ _{—} | _ _ _{—} | 31.0 |
| wild-type control 1 | | | | |
| WT43 | _ _ _{—} | _ _ _{—} | _ _ _{—} | 32.0 |
| wild-type control 2 | | | | |

¹Symbol “X” denotes complete editing of the chromosomal allele of the gene and “_” denotes the wild-type sequence of the chromosomal allele of the gene.
* Line 17-3919 was not analyzed for protein content due to limited seeds for this line.

### Example 5. Additional Gene Targets to Increase Seed Yield, CCP1 Gene from Chlamydomonas reinhardtii

If there is not enough carbon available to the plant to both increase seed yield and increase oil content, a gene target that increases seed yield may help solve this problem. The ccp1 gene encoding the CCP1 protein (SEQ ID NO: 17) is from the algal species Chlamydomonas reinhardtii and has been shown to increase Camelina seed yield when expressed in Camelina using either a constitutive promoter (U.S. Pat. No. 10,337,024) or a seed specific promoter (PCT/US2018/019105). Constitutive expression of CCP1 in Camelina produces an increase in total seed weight per plant. This increase is due to the production of more seeds albeit smaller seeds. In contrast, seed specific expression produces an increase in total seed weight per plant consisting of seeds with higher individual seed weight.

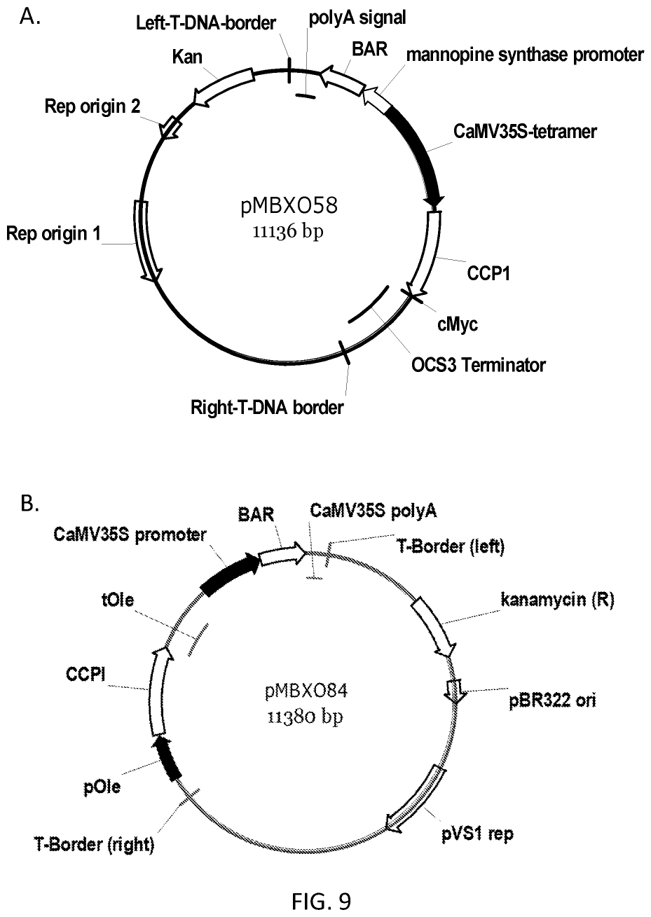

Plasmid pMBXO58 (SEQ ID NO: 18, FIG. 9(A)) contains the gene encoding the CCP1 protein (SEQ ID NO: 17) behind the constitutive 35S promoter. This plasmid can be transformed into lines with sdp1, sdp1-like, and tt2 edits, such as those described in TABLE 14, using the Camelina Agrobacterium-mediated floral dip procedures described above. Transformed T1 seeds can be selected for resistance to bialophos, which is provided to the plant with the presence of an expression cassette for the BAR gene on the T-DNA, and T1 lines can be grown in a greenhouse to produce T2 seeds. Homozygous lines, preferably with single inserts of the T-DNA containing the CCP1 expression cassette, are isolated and grown in a randomized complete block design in a greenhouse with supplemental lighting. Bulk seed yield, bulk seed oil content, the milligrams of oil produced per individual seed, and bulk seed protein content are determined.

Plasmid pMBXO84 (SEQ ID NO: 19, FIG. 9(B)) contains the gene encoding the CCP1 protein (SEQ ID NO: 17) behind the seed specific oleosin promoter from soybean. This plasmid can be transformed into lines with sdp1, sdp1-like, and tt2 edits, such as those described in TABLE 14, using the Camelina Agrobacterium-mediated floral dip procedures described above. Transformed T1 seeds can be selected for resistance to bialophos, which is provided to the plant with the presence of an expression cassette for the BAR gene on the T-DNA, and T1 lines grown in a greenhouse to produce T2 seeds. Homozygous lines, preferably with single inserts of the T-DNA containing the CCP1 expression cassette, are isolated and grown in a randomized complete block design in a greenhouse with supplemental lighting. Bulk seed yield, bulk seed oil content, the milligrams of oil produced per individual seed, and bulk seed protein content are determined.

### Example 6. Additional Gene Targets to Increase Seed Yield, Transcription Factors Identified Using Transcriptome Based Gene Co-Expression Networks

Transcription factors control the transcription and thus the expression of multiple genes in a plant. Transcriptome based gene co-expression networks can be used to identify transcription factors that may control a trait such as seed yield and/or oil content. Yield10 patent application U.S. Provisional Appl. No. 62/873,018, filed Jul. 11, 2019, describes the identification of transcription factors related to seed yield and oil content and is incorporated by reference in its entirety. The genes identified in the Yield10 patent application U.S. Provisional Appl. No. 62/873,018 can be engineered into lines with sdp1, sdp1-like, and tt2 edits, such as those described in TABLE 14, using either gene insertion or genome editing, depending on the target. Preferred transcription factor genes include those listed in TABLE 16.

TABLE 16
Preferred transcription factor genes for engineering
into sdp1, sdp1-like, and tt2 edited lines of Camelina
to increase seed yield and/or oil content.

| | | Gene | Encoded protein |
|---|---|---|---|
| Gene Name | Csa locus | SEQ ID NO | SEQ ID NO |
| LBD42 | Csa16g028530 | SEQ ID NO: 20 | SEQ ID NO: 21 |
| PEI1(also known | Csa13g009540 | SEQ ID NO: 22 | SEQ ID NO: 23 |
| as ATTZF6) | | | |
| DOF4.4 | Csa10g022470 | SEQ ID NO: 24 | SEQ ID NO: 25 |
| ARR21 | Csa20g009570 | SEQ ID NO: 26 | SEQ ID NO: 27 |

### Example 7. Additional Gene Editing Targets to Increase Oil Content

BADC encodes the Biotin/lipoyl Attachment Domain Containing protein, which has been found to be a negative regulator of acetyl-CoA carboxylase (ACCase, Salie et al., Plant Cell, 2016, 28, 2312), the first committed step in de novo fatty acid biosynthesis (FIG. 1). One or more of the Camelina BADC homeologs can be edited into Camelina lines containing the sdp1, sdp1-like, and tt2 edits, such as those described in TABLE 14. With reference to PCT/US2016/041386 to University of Missouri (published as WO 2017/039834), the one or more BADC homeolog edits may serve to increase carbon flow into fatty acid biosynthesis further increasing oil content.

The Camelina genome was searched for BADC orthologs using the Arabidopsis BADC protein sequences as BLAST queries. Nine BADC genes were identified in the Camelina genome and are listed in TABLE 17. These include three orthologs each to the Arabidopsis BADC1, BADC2, and BADC3 genes. Guide sequences for constructing editing constructs to edit the BADC genes are shown in TABLE 18.

TABLE 17
BADC Genes in Arabidopsis and Camelina.

| Arabidopsis | Target Gene | | | | | |
|---|---|---|---|---|---|---|
| gene | (Chromosome | GenBank | GenBank | | | CDS |
| (Arabidopsis | location; SEQ | Gene | Protein | | GenBank | size |
| gene locus) | ID NO) | Accession* | Accession | DH55 LOC No. | annotation | (bp) |
| AtBADC1 | CsBADC1 | XM_010506195.2 | XP_010504497.1 | LOC104781505 | BCCP-like | 831 |
| | (Ch 4; SEQ ID | | | | | |
| | NO: 78) | | | | | |
| (AT3G56130) | CsBADC1 | XM_010517912.2 | XP_010516214.1 | LOC104791905 | BCCP-like | 831 |
| | (Ch 6; SEQ ID | | | | | |
| | NO: 79) | | | | | |
| | CsBADC1 | XM_010429119.2 | XP 010427421.1 | LOC104712265 | BCCP-like | 831 |
| | (Ch 9; SEQ ID | | | | | |
| | NO: 80) | | | | | |
| AtBADC2 | CsBADC2 | XM_010481479.2 | XP_010479781.1 | LOC104758587 | BCCP | 831 |
| | (Ch 17; SEQ | | | | | |
| | ID NO: 81) | | | | | |
| (AT1G52670) | CsBADC2 | XM_010463810.2 | XP_010462112.1 | LOC104742768 | BCCP, XI | 810 |
| | (Ch 14; SEQ | | | | | |
| | ID NO: 82) | | | | | |
| | CsBADC2 | XM_010502574.2 | XPO 10500876.1 | LOC104778185 | BCCP, XI | 810 |
| | (Ch 3; SEQ ID | | | | | |
| | NO: 83) | | | | | |
| AtBADC3 | CsBADC3 | XM_010467246.2 | XP_010465548.1 | LOC104745878 | BCCP | 792 |
| | (Ch 15; SEQ | | | | | |
| | ID NO: 84) | | | | | |
| (AT3G15690) | CsBADC3 | XM_010489100.2 | XPO 10487402.1 | LOC104765401 | BCCP | 792 |
| | (Ch 19: SEQ | | | | | |
| | ID NO: 85) | | | | | |
| | CsBADC3 | XM_010505032.2 | XP_010503334.1 | LOC104780528 | BCCP | 792 |
| | (Ch 1; | | | | | |
| | SEQ ID NO: | | | | | |
| | 86) | | | | | |

*GenBank sequence data is from Camelina line DH55 mR NA

TABLE 18
Guide sequences for editing BADC genes in Camelina.

| | | Guide target | | |
|---|---|---|---|---|
| Guide name | Target Gene | Strand | Guide target sequence (5′ to 3′) | PAM |
| CsC1-69 | CsBADC1-1 | - | GGTTGTTGTCGAAGTTTTAG | AGG |
| | CsBADC1-2 | | (SEQ ID NO: 112) | |
| | CsBADC1-3 | | | |
| CsC2-33 | CsBADC2-1 | + | GCTCATTCCCAAGTCCTCTG | AGG |
| | CsBADC2-2 | | (SEQ ID NO: 113) | |
| | CsBADC2-3 | | | |
| CsC3-52 | CsBADC3-1 | - | GATCCCTTGCTACATATAGG | CGG |
| | CsBADC3-2 | | (SEQ ID NO: 114) | |
| | CsBADC3-3 | | | |
| C1-29A | CsBADC1-3 | + | GTACTTCTTGTGTACCACGG | TGG |
| | | | (SEQ ID NO: 115) | |
| C1-29B | CsBADC1-1 | + | GTACTTCTTGCGTTCCACGG | TGG |
| | CsBADC1-2 | | (SEQ ID NO: 116) | |

Lines containing the sdp1, sdp1-like, and tt2 edits, edits of one or more of the BADC homeologs, and overexpression of one or more of the transcription factors described in TABLE 16, can be engineered to further increase seed yield and seed oil content in Camelina. The sdp1, sdp1-like, tt2, and badc edits are designed to reduce or eliminate the activity of the encoded enzyme. The overexpression of one or more of the transcription factors described in TABLE 16 can be achieved either through gene insertion or genome editing, depending on the target, to increase the expression of the gene.

### Example 8. Editing of sdp1, sdp1-Like, and tt2 Genes in Canola

GenBank was searched for genes annotated as sdp1, sdp1-like, or tt2 in the Brassica napus cv. ZS11 genome and by using the GenBank BLAST search tool using the Arabidopsis SDP1 and SDP1-like proteins as queries. Candidate genes that were identified are listed in TABLE 19.

TABLE 19
SDP1, SDP1-like, and TT2 sequences identified in Brassica napus.

| | | | | | Protein |
|---|---|---|---|---|---|
| | | | | | Length |
| Gene IDs | Gene | Genbank | | Protein ID | (amino |
| (SEQ ID NO:) | Name² | Description | Location¹ | (SEQ ID NO:) | acids) |
| LOC106428475 | BnSDP1-1 | triacylglycerol | Ch A3 | XP_013724700.2 | 783 |
| XM_013869246.2 | | lipase SDP1 | | (SEQ ID NO: 39) | |
| (SEQ ID NO: 38) | | | | | |
| LOC106372666 | BnSDP1-2 | triacylglycerol | Ch A10 | XP_022549517.1 | 822 |
| XM_022693796.1 | | lipase SDP1- | | (SEQ ID NO: 41) | |
| (SEQ ID NO: 40) | | like | | XP_013668385.1 | |
| | | | | XP_022549516.1 | |
| | | | | (3 accessions have | |
| | | | | same protein sequence) | |
| LOC106424050 | BnSDP1-3 | triacylglycerol | Ch A2 | XP_022553589.1 | 806 |
| XM_022697868.1 | | lipase SDP1- | | (SEQ ID NO: 43) | |
| (SEQ ID NO: 42) | | like | | XP_013720243.1 | |
| | | | | XP_022553591.1 | |
| | | | | (3 accessions have | |
| | | | | same protein sequence) | |
| LOC111204220 | BnSDP1-4 | triacylglycerol | Ch C3 | XP_022554181.1 | 760 |
| XM_022698460.1 | | lipase SDP1- | | (SEQ ID NO: 45) | |
| (SEQ ID NO: 44) | | like | | | |
| LOC106372515 | BnSDP1-5 | triacylglycerol | Ch C9 | XP_013668194.1 | 820 |
| XM_013812740.2 | | lipase SDP1; | | (SEQ ID NO: 47) | |
| (SEQ ID NO: 46) | | Sugar- | | | |
| | | Dependent 1 | | | |
| | | like lipase | | | |
| LOC106424215 | BnSDP1-6 | triacylglycerol | Ch C4 | XP_013720422.1 | 340 |
| XM_013864968.2 | | lipase SDP1- | | (SEQ ID NO: 49) | |
| (SEQ ID NO: 48) | | like | | | |
| LOC106377451 | BnSDP1-7 | triacylglycerol | Unplaced | XP_013673142.1 | 806 |
| XM_013817688 | | lipase SDP1- | Scaffold | (SEQ ID NO: 51) | |
| (SEQ ID NO: 50) | | like | | XP_022568536.1 | |
| | | | | (2 accessions have | |
| | | | | same protein sequence) | |
| LOC106410713 | BnSDP1 | Sugar- | Ch C6 | XP_022560988.1 | 785 |
| XM_022705267.1 | like-1 | Dependent 1 | | (SEQ ID NO: 53) | |
| (SEQ ID NO: 52) | | like lipase | | XP_013706737.1 | |
| | | | | (2 accessions have | |
| | | | | same protein sequence) | |
| LOC106356852 | BnSDP1 | triacylglycerol | Ch A3 | XP_013652028.1 | 786 |
| XM_013796574.2 | like-2 | lipase SDP1L- | | (SEQ ID NO: 55) | |
| (SEQ ID NO: 54) | | like | | | |
| LOC106445914 | BnSDP1 | triacylglycerol | Ch A8 | XP_022545673.1 | 786 |
| XM_022689952.1 | like-3 | lipase SDP1L- | | (SEQ ID NO: 57) | |
| (SEQ ID NO: 56) | | like | | XP_013743023.1 | |
| | | | | (2 accessions have | |
| | | | | same protein sequence) | |
| LOC106389096 | BnSDP1 | triacylglycerol | Unplaced | XP_022568746.1 | 785 |
| XM_022713025.1 | like-4 | lipase SDP1L- | Scaffold | (SEQ ID NO: 59) | |
| (SEQ ID NO: 58) | | like | | XP_013684745.1 | |
| XM_013829291.2 | | | | (SEQ ID NO: 60) | |
| (SEQ ID NO: 61) | | | | (2 accessions have | |
| | | | | different | |
| | | | | protein sequence | |
| LOC106418890 | BnTT2-1 | transcription | Ch A8 | NP_001303110.1 | 260 |
| NM_001316181.1 | | factor TT2-like | | (SEQ ID NO: 63) | |
| (SEQ ID NO: 62) | | | | | |
| LOC106359998 | BnTT2-2a | transcription | Unplaced | XP_013655061.1 | 260 |
| XM_013799607.2 | | factor TT2-like | Scaffold | (SEQ ID NO: 65) | |
| (SEQ ID NO: 64) | | isoform X2 | | | |
| LOC106359998 | BnTT2-2b | transcription | Unplaced | XP_022566873.1 | 275 |
| XM_022711152.1 | | factor TT2-like | Scaffold | (SEQ ID NO: 67) | |
| (SEQ ID NO: 66) | | isoform X1 | | | |
| LOC106359997 | BnTT2-3a | transcription | Unplaced | XP_013655060.1 | 260 |
| XM_013799606.2 | | factor TT2-like | Scaffold | (SEQ ID NO: 69) | |
| (SEQ ID NO: 68) | | isoform X2 | | | |
| LOC106359997 | BnTT2-3b | transcription | Unplaced | XP_022566879.1 | 275 |
| XM_022711158.1 | | factor TT2-like | Scaffold | (SEQ ID NO: 71) | |
| (SEQ ID NO: 70) | | isoform X1 | | | |
| LOC106359996 | BnTT2-4a | transcription | Unplaced | XP_013655059.1 | 260 |
| XM_013799605.2 | | factor TT2 | Scaffold | (SEQ ID NO: 73) | |
| (SEQ ID NO: 72) | | isoform X2 | | | |
| LOC106359996 | BnTT2-4b | transcription | Unplaced | XP_022566888.1 | 275 |
| XM_022711167.1 | | factor TT2 | Scaffold | (SEQ ID NO: 75) | |
| (SEQ ID NO: 74) | | isoform X1 | | | |
| LOC106418785 | BnTT2-5 | transcription | Ch A8 | XP_013714993.1 | 141 |
| XM_013859539.2 | | factor TT2-like | | | |
| (SEQ ID NO: 76) | | | | | |

¹Abbreviation: Ch, chromosome.
²In the present work, the annotation of the gene as sdp1, sdp1-like, or tt2 was based on sequence similarity analysis, as well as syntenic analysis.
²SEQ ID NO: 58 and SEQ ID NO: 60 are two transcripts predicted from the same locus that yield different protein sequences.
*SEQ ID NO: 58 and SEQ ID NO: 61 are two transcripts predicted from the same locus that yield different protein sequences.

The canola orthologs of BADC are identified using the Arabidopsis genes. Canola lines containing the sdp1, sdp1-like, and tt2 edits, and edits of one or more of the canola BADC homeologs, can be engineered to further increase seed yield and seed oil content in canola. The sdp1, sdp1-like, tt2, and badc edits are designed to reduce or eliminate the activity of the encoded enzyme.

Canola lines containing the sdp1, sdp1-like, and tt2 edits, and edits of one or more of the canola BADC homeologs, can be engineered to further increase seed yield and seed oil content in Canola. The sdp1, sdp1-like, tt2, and badc edits are designed to reduce or eliminate the activity of the encoded enzyme.

To increase seed yield, one or more of the canola orthologs of the Camelina transcription factors described in TABLE 16 can be overexpressed in canola lines containing sdp1, sdp1-like, tt2, or badc edits, either through gene insertion or genome editing, depending on the target.

Alternatively, to increase seed yield, expression constructs for the ccp1 gene encoding the CCP1 protein from Chlamydomonas reinhardtii (SEQ ID NO: 17) can be transformed into canola lines containing sdp1, sdp1-like, tt2, or badc edits. A construct for constitutive expression of CCP1, such as pMBXO58 (SEQ ID NO: 18) that has the ccp1 gene under the control of the 35S promoter, or seed specific expression of CCP1, such as pMBXO84 (SEQ ID NO: 19), can be used.

### Example 9. Editing of sdp1, sdp1-Like, and tt2 Genes in Soybean

GenBank was searched for genes annotated as sdp1, sdp1-like, or tt2 in the Glycine max cv. Williams 82 genome and by using the GenBank BLAST search tool using the Arabidopsis proteins as queries. Candidate genes that were identified are listed in TABLE 20. The soybean SDP1 and SDP1-like orthologs could not be distinguished from each other since their sequence similarities were very close. Thus all SDP1 or SDP1-like candidates in TABLE 20 are referred to generally as GmSDP1-1 through GmSDP1-4. These four soybean orthologs have previously been identified and characterized using RNA interference to investigate their role during grain filling (Kanai et al., 2019, Scientific Reports, 9, 8924). Knockdown of all four SDP1 genes lead to increased seed oil content and a modified fatty acid profile for the oil. It is an object of this invention to further improve soybean by combining genome edits in the GmSDP1-1 through GmSDP1-4 genes with edits in one or more of the soybean tt2 genes to increase the flow of carbon through fatty acid biosynthetic pathways.

TABLE 20
SDP1 and TT2 sequences identified in Glycine max cv. Williams 82.

| | | | | | | Protein |
|---|---|---|---|---|---|---|
| | | | | | | Length |
| Gene IDs | Gene | Genbank | | Gene ID | Protein ID | (amino |
| (SEQ ID NO:) | Name¹ | Description | Location | (SEQ ID NO:) | (SEQ ID NO:) | acids) |
| LOC100817268 | GmSDP1-1 | triacylglycerol | Ch 2 | XM_014768280.2² | XP_014623766.1 | 805 |
| | | | | (SEQ ID NO: 87) | (SEQ ID NO: 98) | |
| | | lipase SDP1 | | XM_014768281.2² | XP_014623767.1 | 758 |
| | | | | (SEQ ID NO: 88) | (SEQ ID NO: 99) | |
| | | isoform | | XM_026125950.1² | XP_025981735.1 | 799 |
| | | | | (SEQ ID NO: 89) | (SEQ ID NO: 100) | |
| LOC100807526 | GmSDP1-2 | triacylglycerol | Ch 10 | XM_006588902.3² | XP 006588965.1 | 804 |
| | | isoform | | (SEQ ID NO: 90) | (SEQ ID NO: 101) | |
| | | lipase SDP1 | | XM_003537223.4² | XP_003537271.1 | 854 |
| | | | | (SEQ ID NO: 91) | (SEQ ID NO: 102) | |
| LOC100816093 | GmSDP1-3 | triacylglycerol | Ch 19 | XM_003554093.3 | XP_003554141.1 | 840 |
| | | lipase SDP1 | | (SEQ ID NO: 92) | (SEQ ID NO: 103) | |
| LOC100791261 | GmSDP1-4 | triacylglycerol | Ch 3 | XM_003521103.4 | XP_003521151.2 | 844 |
| | | lipase SDP1 | | (SEQ ID NO: 93) | (SEQ ID NO: 104) | |
| LOC100809225 | GmTT2-1 | transcription | Ch 16 | XM_003548315.4 | XP_003548363.1 | 285 |
| | | factor TT2 | | (SEQ ID NO: 94) | (SEQ ID NO: 105) | |
| LOC100794570 | GmTT2-2 | transcription | Ch 10 | XM_003535467.4 | XP_003535515.1 | 273 |
| | | factor TT2 | | (SEQ ID NO: 95) | (SEQ ID NO: 106) | |
| LOC547568 | GmTT2-3 | transcription | Ch 16 | XM_026126004.1 | XP_025981789.1 | 261 |
| | | factor TT2; | | (SEQ ID NO: 96) | (SEQ ID NO: 107) | |
| | | transcription | | | | |
| | | repressor MYB6 | | | | |
| LOC100802704 | GmTT2-4 | transcription | Ch 17 | NM_001355658.1 | NP_001342587.1 | 307 |
| | | factor MYB205; | | (SEQ ID NO: 97) | (SEQ ID NO: 108) | |
| | | Note: | | | | |
| | | transcription | | | | |
| | | factor TT2-like | | | | |

¹The soybean SDP1 and SDP1-like orthologs could not be distinguished from each other since their sequence similarities are very close. Thus all SDP1 or SDPl-like candidates in TABLE 20 are referred to generally as GmSDP1-1 through GmSDP1-4.
²Multiple transcript variants were identified from the same genomic locus in the current annotation version of the soybean Williams 82 genome in GenBank.

Soybean orthologs of BADC have been previously described (see PCT/US2016/041386 to University of Missouri (published as WO 2017/039834)). Soybean lines containing the sdp1, sdp1-like, and tt2 edits, and edits of one or more of the soybean BADC homeologs, can be engineered to further increase seed yield and seed oil content in soybean. The sdp1, sdp1-like, tt2, and badc edits are designed to reduce or eliminate the activity of the encoded enzyme.

To increase seed yield, one or more of the soybean orthologs of the Camelina transcription factors described in TABLE 16 can be overexpressed in soybean lines containing sdp1, sdp1-like, tt2, or badc edits, either through gene insertion or genome editing, depending on the target.

Alternatively, to increase seed yield, expression constructs for the ccp1 gene encoding the CCP1 protein from Chlamydomonas reinhardtii (SEQ ID NO: 17) can be transformed into soybean lines containing sdp1, sdp1-like, tt2, or badc edits. A construct for constitutive expression of CCP1 in soybean containing for example, the 35S promoter, can be transformed into soybean lines containing one or more of the sdp1, sdp1-like, tt2, or badc edits. Alternatively, a genetic construct expressing the ccp1 gene under the control of a seed specific promoter, such as the soybean oleosin promoter, can be used.

### Example 10. Results for Camelina Line 17-3902 and Wild-Type Control in Small Scale Replicated Field Plots

Camelina line 17-3902 (Table 14) and wild-type controls were planted in the spring of 2019 in small scale replicated field plots in Idaho. Plots were 6 m² by 1.24 m² and were replicated 6 times. Plots were harvested and analyzed for yield, seed oil content, and individual seed weight. A 4.7% increase in the bulk seed oil content (% of seed weight) was observed compared to the oil content in the wild-type control line (TABLE 21). In addition, an increased seed yield of 9.7% was observed. Individual seeds were found to be heavier (8.7%) and contained more oil (11.8%) compared to individual control wild-type seeds. Based on these values, a 15% increase in total oil produced per hectare was calculated.

TABLE 21
Results from small scale replicated field plots of line 17-3902 and wild-type control.

| % Increase, | | | | | |
|---|---|---|---|---|---|
| oil per | % Increase, | % Increase, | | | % Increase, |
| individual | individual | seed yield | % Increase, | % Increase, | total oil |
| seed | seed weight | (kg seed per | seed oil content | number of seeds | produced per |
| (mgs) | (mgs) | hectare) | (% of seed weight) | harvested | hectare |
| 11.8* | 8.7* | 9.7 | 4.7* | −3.7 | 15.0 |

*statistically significant (t-test)

The mutations in SDP1 and SDP1-like triacylglycerol lipases may prevent the degradation of oil in mature seeds, reducing the amount of free fatty acids present in oil. Free fatty acids are not desirable in oil since they may give the oil an unpleasant taste. Since some residual oil is still present in seed meal used for animal or fish feeds, lower free fatty acids may improve the palatability of Camelina meal used in feed. The levels of free fatty acids in oil extracted from seeds from line 17-3902 can be measured using the American Oil Chemists' Society standardized method AOCS Ac-541 and compared to levels in oil extracted from wild-type seeds.

The presence of the tt2 mutation in line 17-3902 may also lower fiber content in seeds, which may improve the digestibility of Camelina meal used as animal feed. Fiber content in seeds can be measured using standard methods for generation of acid detergent fiber (ADF). Holtzapple describes standardized methods for preparing and measuring acid detergent fiber from plant material (M. T. Holtzapple, in Encyclopedia of Foods Sciences and Nutrition, Editors. Luiz Trugo and Paul M. Finglas, Second Edition, 2003) which can be used in this invention to measure acid detergent fiber.

Protein content, amino acid composition, and starch content of the seeds can also be measured using standard techniques available from contract laboratories.

REFERENCE TO A “SEQUENCE LISTING,” A TABLE, OR A COMPUTER PROGRAM LISTING APPENDIX SUBMITTED AS AN ASCII TEXT FILE

The material in the ASCII text file, named “YTEN-61224WO-Sequence-Listing_ST25.txt”, created Jul. 16, 2020, file size of 442,368 bytes, is hereby incorporated by reference.

What is claimed is:

1. A genetically modified plant that exhibits an increase in seed yield relative to a progenitor plant from which the genetically modified plant was derived, the genetically modified plant comprising:
(a) a first homeolog of the SUGAR-DEPENDENT1 (SDP1) gene, occurring in its natural position within the genome of the genetically modified plant and being homozygous for a wild-type allele; and
(b) a second homeolog of the SDP1 gene, occurring in its natural position within the genome of the genetically modified plant and being homozygous for a mutant allele, wherein:
(i) the wild-type allele encodes an active SDP1 triacylglycerol lipase and is identical to an allele of the first homeolog of the SDP1 gene from the progenitor plant;
(ii) the mutant allele does not encode an active SDP1 triacylglycerol lipase and includes one or more additions, deletions, or substitutions of one or more nucleotides relative to an allele of the second homeolog of the SDP 1 gene from the progenitor plant;
(iii) the genetically modified plant expresses about 20% to 80% of SDP1 triacylglycerol lipase activity in seeds relative to the progenitor; and
(iv) the increase in seed yield is at least 10%.

2. The genetically modified plant of claim 1, wherein the genetically modified plant comprises the first homeolog and the second homeolog based on one or more of polyploidy, alloploidy, autoploidy, diploidization following polyploidy, diploidization following alloploidy, or diploidization following autoploidy.

3. The genetically modified plant of claim 1, wherein the genetically modified plant is allotetetraploid, allohexaploid, or allooctoploid.

4. The genetically modified plant of claim 1, wherein the genetically modified plant is homozygous for the wild-type allele based on including two identical copies of a wild-type allele.

5. The genetically modified plant of claim 1, wherein the genetically modified plant is homozygous for the wild-type allele based on including a first wild-type allele and a second wild-type allele that are not identical to each other.

6. The genetically modified plant of claim 1, wherein the genetically modified plant is homozygous for the mutant allele based on including two copies of the mutant allele that are identical.

7. The genetically modified plant of claim 1, wherein the genetically modified plant is homozygous for the mutant allele based on including a first mutant allele and a second mutant allele that are not identical to each other.

8. The genetically modified plant of claim 1, wherein the active SDP1 triacylglycerol lipase has a sequence that is at least 70% identical to one or more SEQ ID NO: 30, SEQ ID NO: 31, or SEQ ID NO: 32.

9. The genetically modified plant of claim 8, wherein the active SDP1 triacylglycerol lipase has a sequence that comprises SEQ ID NO: 30, SEQ ID NO: 31, or SEQ ID NO: 32.

10. The genetically modified plant of claim 1, wherein the one or more additions, deletions, or substitutions of one or more nucleotides comprise one or more of a frameshift mutation, an active site mutation, a nonconservative substitution mutation, or an open-reading-frame deletion mutation in the mutant allele relative to the allele of the second homeolog of the SDP1 gene from the progenitor plant.

11. The genetically modified plant of claim 1, wherein the genetically modified plant expresses about 30% to 70% of SDP1 triacylglycerol lipase activity in seeds relative to the progenitor.

12. The genetically modified plant of claim 1, wherein the increase in seed yield is at least 20%, 25%, 30%, 35%, 40%, 45%, 50%, or more.

13. The genetically modified plant of claim 1, further comprising a third homeolog of the SDP1 gene occurring in its natural position within the genome of the genetically modified plant.

14. The genetically modified plant of claim 13, wherein the third homeolog is homozygous for a wild-type allele.

15. The genetically modified plant of claim 13, wherein the third homeolog is homozygous for a mutant allele.

16. The genetically modified plant of claim 13, wherein the third homeolog is heterozygous for a wild-type allele and a mutant allele.

17. The genetically modified plant of claim 1, further comprising:
(a) a first homeolog of the SUGAR-DEPENDENT1-LIKE (SDP1-L) gene, occurring in its natural position within the genome of the genetically modified plant and being homozygous for a wild-type allele; and
(b) a second homeolog of the SDP1-L gene, occurring in its natural position within the genome of the genetically modified plant and being homozygous for a mutant allele, wherein:
(i) the wild-type allele encodes an active SDP1-L triacylglycerol lipase and is identical to an allele of the first homeolog of the SDP1-L gene from the progenitor plant; and
(ii) the mutant allele does not encode an active SDP1-L triacylglycerol lipase and includes one or more additions, deletions, or substitutions of one or more nucleotides relative to an allele of the second homeolog of the SDP1-L gene from the progenitor plant.

18. The genetically modified plant of claim 1, further comprising:
(a) a first homeolog of the TRANSPARENT TESTA2 (TT2) gene, occurring in its natural position within the genome of the genetically modified plant and being homozygous for a wild-type allele; and
(b) a second homeolog of the TT2 gene, occurring in its natural position within the genome of the genetically modified plant and being homozygous for a mutant allele, wherein:
(i) the wild-type allele encodes an active TT2 transcription factor and is identical to an allele of the first homeolog of the TT2 gene from the progenitor plant; and
(ii) the mutant allele does not encode an active TT2 transcription factor and includes one or more additions, deletions, or substitutions of one or more nucleotides relative to an allele of the second homeolog of the TT2 gene from the progenitor plant.

19. The genetically modified plant of claim 1, wherein the genetically modified plant is one or more of a Brassica species, Brassica napus, Brassica rapa, Brassica carinata, Brassica juncea, Camelina sativa, a Crambe species, a Jatropha species, pennycress, Ricinus communis, a Calendula species, a Cuphea species, Arabidopsis thaliana, maize, soybean, a Gossypium species, sunflower, palm, coconut, safflower, peanut, Sinapis alba, sugarcane, flax, or tobacco.

20. The genetically modified plant of claim 19, wherein the genetically modified plant is Brassica napus, Brassica rapa, Brassica carinata, Brassica juncea, Camelina sativa, or soybean.

21. The genetically modified plant of claim 1, wherein the genetically modified plant is Camelina sativa.

22. The genetically modified plant of claim 21, wherein the natural position of the second homeolog of the SDP1 gene is on chromosome 13 of Camelina sativa.

23. The genetically modified plant of claim 21, wherein the allele of the second homeolog of the SDP1 gene from the progenitor plant encodes a protein that has a sequence comprising SEQ ID NO: 31.

24. The genetically modified plant of claim 21, wherein the allele of the second homeolog of the SDP1 gene from the progenitor plant comprises SEQ ID NO: 2.

25. The genetically modified plant of claim 21, further comprising a third homeolog of the SDP1 gene occurring in its natural position within the genome of the genetically modified plant, wherein the third homeolog is homozygous for a wild-type allele.
